(19) United States
(12) Patent Application Publication
(10) Pub. No.: US 2025/0052762 A1
(43) Pub. Date: Feb. 13, 2025

(54) PEPTIDASE CLEAVABLE SUBSTRATES AND METHODS OF IDENTIFICATION AND USE THEREOF

(71) Applicant: Alaunus Biosciences, Inc., San Francisco, CA (US)

(72) Inventors: Giselle M. Knudsen, San Anselmo, IN (US); Mark D. Cisneros, West Lafayette, IN (US)

(21) Appl. No.: 18/268,897

(22) PCT Filed: Dec. 21, 2021
(86) PCT No.: PCT/US21/64623

§ 371 Date: Jun. 21, 2023

Related U.S. Application Data
(60) Provisional application No. 63/128,475, filed on Dec. 21, 2020.

(51) Int. Cl. G01N 33/68; G16B 35/00

(52) U.S. Cl. CPC G01N 33/6845; G16B 35/00; G01N 2333/95

(57) ABSTRACT

The present disclosure relates to library compositions, methods of making and screening libraries of peptidase-cleavable substrate sequences. In particular, the libraries are useful in identifying substrate sequences for a variety of disease- or tissue-specific peptidases. The present disclosure also relates to use of identified peptidase substrates to design therapeutic agents and diagnostic tools.

The present application claims the benefit of priority of U.S. provisional application No. 63/128,475, filed Dec. 21, 2020, which is incorporated by reference herein in its entirety.

### FIELD

The present disclosure is directed to methods to generate and screen libraries of peptidase cleavable substrates. In particular, the libraries are useful in identifying substrate profiles for a variety of disease-, tissue- or cell-selective peptidases. The present disclosure also relates to use of identified peptidase substrates to design therapeutic agents and diagnostic tools.

### BACKGROUND

Peptidases occur naturally in all organisms and are involved in a variety of physiological reactions from simple degradation to highly regulated pathways. The defining feature of a peptidase is its ability to cleave a polypeptide substrate through the chemical process of hydrolysis (Enzyme nomenclature: Recommendations (1992) of the Nomenclature Committee of the International Union of Biochemistry and Molecular Biology. pp 862. Academic Press, San Diego. Biochemical Education 21, 102 (1993)). Similar to oligonucleotide sequences that are cleaved by restriction enzymes, “peptidase substrates” are described by strings of letters representing the amino acid sequence that is recognized and cleaved by the enzyme, in a “substrate sequence”.

The specificity of a peptidase for its substrate(s) is dictated by complex interactions of exosites to capture and appropriately orient the substrate with the active site, which catalyzes peptide bond cleavage through hydrolysis. Since peptidases have important roles in controlling physiological signaling through their regulation of bioactive peptides, understanding and controlling the interplay between peptidases and their substrates is of great biomedical interest. Therefore, discovery of peptide substrates that are selectively cleaved by peptidases which are active at the site of disease or a specific tissue can pave the way for the development of novel diagnostic tools and pharmaceutical agents.

Many approaches have been developed to reveal the substrate specificity of peptidases and discover peptidase substrates. The most common method used to identify peptidase substrates is a “candidate-based” approach, where a candidate substrate to be tested for cleavage is treated with a peptidase under controlled reaction conditions, and the disappearance of substrate and formation of products are monitored using molecular detection methods such as fluorescence spectroscopy, immuno-detection, or mass spectrometry. Candidate-based methods historically have examined cleavage patterns within generic peptides or proteins such as insulin A or B chains, immunoglobulins or collagen (Ryle, A. P. et al., Biochemical Journal 73, 75-86 (1959); Powers. J. C. et al., Adv. Exp. Med. Biol. 95, 141-157 (1977)), or in more modern studies they have focused on small molecule substrates bearing substrate sequences that were predicted from prior knowledge for the peptidase (Cs-Szabo, G. et al., Thrombosis Research 20, 199-206 (1980); Clavin, S. A. et al., Analytical Biochemistry 80, 355-365 (1977)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues; immunologically tagged proteins; and the like.

The methods for peptidase substrate discovery using library-based screens of substrate sequences fall into two main approaches that combine either “positional scanning” or “combinatorial” library design with various detection and separation methods for monitoring substrate cleavage in pooled samples.

A “positional scanning” approach for library design can be used to vary as few as one position at a time within a candidate substrate sequence, while holding other positions constant. The positional scanning-synthetic combinatorial library (PS-SCL) method employs synthetic peptide substrate libraries that contain a C-terminal fluorescent tag that produces fluorescence signal upon cleavage, i.e. “fluorogenic” substrates (Rano, T. A. et al., Chem. Biol., vol. 4, no. 2, p. 149-155 (1997)). Since fluorescence detection cannot distinguish multiple substrates in a pooled sample, libraries of PS-SCL substrates are synthesized and assayed in subsets that are defined by sharing one or more fixed amino acids at one or more positions within the substrate sequence. Thus, cleavage is not determined for individual, unique substrates, but instead it is measured as an average value for that subset of the library. PS-SCL and related fluorescent peptide substrate methods are also limited in the amino acid positions within a substrate sequence that can be tested and the types of peptidases that will accept these substrates (Ivry, S. L. et al., Protein Science 27, 584-594 (2018)). Partial substrate specificity information has been obtained by PS-SCL for peptidases that include caspases, cathepsins, legumain, plasmin, thrombin, hepsin, urokinase plasminogen activator (uPA) and tissue plasminogen activator (IPA) (Harris, J. L. et al., PNAS 97, 7754-7759 (2000); Herter, S. et al., Biochem J 390, 125-136 (2005); Choe, Y. et al., J. Biol. Chem. 281, 12824-12832 (2006); Thornberry, N. A. et al., J. Biol. Chem. 272, 17907-17911 (1997)).

Alternatively, “combinatorial library” designs vary the amino acids at multiple positions simultaneously within a substrate sequence, and are typically synthesized using random incorporation of amino acids at each varied position. The library is assayed in a single pooled reaction, and the most readily cleaved substrates are identified. “Combinatorial display libraries” containing approximately 10⁷ to 10¹⁰ unique substrate sequences have been produced in microorganisms and cellular display systems, by inserting the library of candidate peptidase substrate sequences into the sequence of a host protein that displays the candidate substrate sequences on the surface of these organisms. Such combinatorial display libraries for the identification of substrate sequences have been produced in filamentous phage (Matthews, D. J. et al., Science 260, 1113-1117 (1993)), or on the cell surface of yeast or bacteria (Georgiou, G. et al., Nature Biotechnology 15, 29-34 (1997)). In practice, after a cleavage assay is performed using peptidase treatment of the library, the cleaved species are separated from the non-cleaved species for quantification and sequence identification. For example, one method for E. coli display (Daugherty, P. S. & Rice, J. U.S. patent Ser. No. 10/640,762-Methods for enhancing bacterial cell display of proteins and peptides. (2020)) uses flow cytometric sorting of clones with fluorescence-based detection of the cleaved products, followed by genomic sequencing to identify the clones bearing the top-most cleaved peptidase substrates (Wittrup, K. D. et al., Current Opinion in Biotechnology 12, 395-399 (2001)). Combinatorial surface display methods have been employed for the identification of specific substrates for the peptidases Caspase-3, MMP14, Factor Xa, enterokinase, uPA and matriptase (U.S. Pat. Nos. 8,293,685; 7,666,817).

The essential problem with these approaches is that they are focused on measuring substrate specificity, yet none of the library-based methods reported to date can both fully cover all of sequence diversity required and accurately measure substrate specificity. This problem arises from inefficiencies in library design and limitations in data collection from the screening method. The mathematical representation of substrate library cleavage data is a matrix of values for the frequency that an amino acid is observed at each position within a substrate sequence; therefore, to be a true frequency, the library must explore the complete sequence diversity at a given position in the sequence, and both cleaved and non-cleaved substrates need to be identified to verify the accuracy of the amino acid frequency measured. The matrix of amino acid frequencies measured from positional scanning and combinatorial library approaches will yield different values, due to the screening methods used, which each produce incomplete information. Furthermore, the identity of the amino acids at additional sites elsewhere in the substrate sequence will influence overall substrate cleavability by the peptidase, a concept referred to as “cooperativity” that is lost or ignored in the typical analysis because the rate of co-occurrence between amino acids in various positions is not considered. This makes the prediction of new substrates purely from an amino acid frequency matrix unreliable, since simply combining the most frequently observed amino acids at each position into a new candidate sequence will not reliably yield a high efficiency substrate sequence.

Thus, there is significant need for a method for designing a substrate sequence library to maximize sequence diversity within the library that directly tests cooperative interactions, allowing for more efficient exploration of the full sequence space available for peptidase substrate design. Identifying peptidase-cleavable substrate sequences within this diverse library is critical to uncover novel therapeutic and diagnostic molecules for disease-, tissue- or cell-selective cleavage.

### SUMMARY

In one aspect, the present invention provides libraries and methods for obtaining an enzymatic substrate profile, such as for determining substrate(s) (sequences) for peptidases. In general, a method for producing an unbiased peptide library comprises i) Hypothesis generation, ii) Discovery; iii) Design and iv) Evaluation of enzyme-cleavable substrates.

In another aspect, methods of making libraries are also provided. As an example, the peptidase substrate libraries disclosed herein are useful in obtaining a complete substrate profile of a peptidase.

In one aspect, a method of constructing an unbiased library of peptides or proteins, comprises: designing a peptide library by a combination of biological assays and computational modeling wherein each peptide comprises at least a set of two, three or more amino acid motifs; generating a plurality of peptides comprising the at least a set of two, three or more amino acid motifs which are variable based on amino acid sequences and positioning of each motif in each peptide; assessing cooperative interactions between at least two, three or more amino acid motifs within a given sequence space for a defined number of amino acid residues and length of a peptide substrate sequence; extracting of amino acid motifs by a computational algorithm by measuring cooperativity between two or more unique amino acids found at specific positions within substrate sequences of a peptide library; thereby constructing an unbiased library of peptides or proteins.

In certain embodiments, in the at least two, three or more amino acid motifs are extracted by a computational algorithm comprising measuring cooperativity between two or more unique amino acids found at specific positions within substrate sequences of a library. In certain embodiments, the at least two, three or more amino acid motifs comprise a minimal set of two or more amino acid positions within a substrate sequence that interacts with one or enzymes. In certain embodiments, the amino acids comprised in a particular amino acid motif have a positive or negative effect upon the rate of scissile bond cleavage by the one or more enzymes. In certain embodiments, the substrate sequence is an enzyme substrate sequence capable of being cleaved by at least one enzyme. In certain embodiments, the at least one enzyme comprises one or more peptidases. In certain embodiments, the one or more peptidases comprise: endopeptidases, omega-peptidases, exopeptidases, aminopeptidases, carboxypeptidases, dipeptidyl-peptidases, tripeptidyl peptidases, peptidyl dipeptidases, dipeptidases, or combinations thereof. In certain aspects, the unbiased library of proteins or peptides is incubated with one or more peptidases to form a population of cleaved and non-cleaved peptides or proteins.

In another aspect, a method of building peptidase cleavable substrate library is provided, the method comprises the steps of:
(i) Providing an unbiased library of peptides or proteins, wherein the peptide or protein comprises a substrate sequence, and the design of the library is the result of mathematical modeling to assess all possible cooperative interactions between at least two, three or more amino acid residues within a given sequence space for a defined number of residues and length of substrate sequence;
(ii) Incubating the library in the presence of the peptidase, allowing the peptidase to cleave peptides or proteins within the library to form a population of cleaved and non-cleaved peptides or proteins;
(iii) Screening the population of cleaved and non-cleaved peptides or proteins for obtaining Gearr™ motifs;
(iv) Extracting Gearr™ motifs using a computational algorithm; and
(v) Building a refined peptidase cleavable substrate sequence library from Gearr™ motifs, wherein the substrate sequences are capable of being cleaved by at least one peptidase.

In another aspect, provided herein is a method of obtaining a peptidase cleavable substrate comprising the steps of:
(i) Building a peptidase cleavable substrate library as described above in this disclosure;
(ii) Selecting individual peptidase cleavable substrate comprising at least one Gearr™ motif, wherein the substrate is capable of being selectively cleaved by at least one, at least two, or at least three peptidases.

In another aspect, provided herein is a method of obtaining disease-, tissue- and/or cell-selective peptidase cleavable substrate, comprising the steps of:
(i) Obtaining a peptidase cleavable substrate as described above in this disclosure;
(ii) Optionally, identifying bracketing residues, wherein bracketing residues are located at upstream and/or downstream positions outside of a candidate disease-, tissue- and/or cell-selective peptidase cleavable substrate;
(iii) Contacting the candidate peptidase cleavable substrate with at least one disease-, tissue- and/or cell-selective peptidase;
(iv) Evaluating and validating cleavage of the candidate substrate sequence, whereby obtaining the disease-, tissue- and/or cell-selective peptidase cleavable substrate.

In some embodiments, obtaining disease-, tissue- and/or cell-selective peptidase cleavable substrate sequence is useful for therapeutic and/or diagnostic applications.

In some embodiments, obtaining disease-, tissue- and/or cell-selective peptidase cleavable substrate sequence is for use in therapeutic agents and/or diagnostic tools.

In certain embodiments, a kit is provided wherein the kit comprises one or more unbiased peptide libraries embodied herein.

In certain embodiments, a peptide library comprising one or more unbiased peptides embodied herein.

Hereinafter, the method of obtaining peptidase cleavable substrate sequence or the method of obtaining disease-, tissue- and/or cell-selective peptidase cleavable substrate sequence will be referred as Alauna™ method.

In one aspect, The Alauna™ method is an approach that efficiently searches sequence space by directly testing cooperativity between sets of amino acids in all positions within candidate peptidase cleavable substrate sequences. Alauna™ method requires fewer substrate sequences in a well-defined library to screen a larger substrate sequence space, overcoming practical library size limitations, and it provides a more efficient method to discover disease-, tissue- and/or cell-selective peptidase cleavable substrate sequences.

In some embodiments, the peptidases are selected from the group of endopeptidases, omega-peptidases, exopeptidases, aminopeptidases, carboxypeptidases, dipeptidyl-peptidases, tripeptidyl peptidases, peptidyl dipeptidases, dipeptidases, or combination thereof.

In some embodiments, the peptidases are extracted from human subject-derived tissues and/or biofluids, cell cultures, transgenic cellular expression systems, or animal models for human disease and biological systems.

In some embodiments, the peptidases are prepared from lysates, extracts, biofluids or conditioned media, with or without further purification.

In some embodiments, the screening of the population of cleaved and non-cleaved substrates comprises quantitively measuring the abundance of cleavage products at different time points by sequencing methods.

In another embodiment, the screening of the population of cleaved and non-cleaved substrates further comprises analysis and identification of the scissile bond for each individual peptide by sequencing methods.

In some embodiments, the sequencing methods are selected from mass spectrometry-based proteomic analysis, differential fluorescence, differential immunodetection, next generation sequencing techniques, or combination thereof.

In some embodiments, extracting Gearr™ motifs using a computational algorithm comprises measuring cooperativity between two or more unique amino acids found at specific positions within the substrate sequences of a library.

In some embodiments, Gearr™ motifs comprise both positive and negative specificity information.

In some embodiments, Gearr™ motifs are useful for obtaining peptidase cleavable substrate. In other embodiments, Gearr™ motifs are useful for obtaining disease-, tissue- and/or cell-selective peptidase cleavable substrate.

In some embodiments, building peptidase cleavable substrate library from Gearr™ motifs comprises testing a candidate peptidase cleavable substrate within a sample, wherein the sample is selected based on a criteria matrix.

In other embodiments, contacting the candidate peptidase cleavable substrate with at least one disease-, tissue- and/or cell-selective peptidase comprises testing a candidate peptidase cleavable substrate within a sample, wherein the sample is selected based on a criteria matrix.

In some embodiments, the criteria matrix guides sample selection.

In some embodiments, the criteria matrix is based on the molecular target for a therapeutic drug design, and the healthy and diseased tissues where the target is expressed.

In some embodiments, the criteria matrix comprises scored measurements for i) co-expression of peptidases with the drug target at the therapeutic site of action versus in other sites, ii) peptidase specificity, iii) maximum peptidase catalytic activity, and iv) specific co-localization of peptidases with the drug target at the therapeutic site of action.

The therapeutic site of action is specified by the disease status and the location of the molecular target in the body with features such as organ, tissue, compartment, cell type, and subcellular localization. The specification for the therapeutic site of action affects sample selection for measurement of protein abundance and peptidolytic activity at that site.

In some embodiments, Alauna™ method comprises a method for substrate sequence library design to maximize sequence diversity within the library that directly tests cooperative interactions, allowing for more efficient exploration of the full sequence space available for peptidase substrate design.

In some embodiments, the Alauna™ method comprises a screening step to achieve accurate substrate specificity information.

In some embodiments, substrate specificity information is analyzed to identify Gearr™ motifs, which are defined herein as the identity of the minimum set of amino acids and their positions within a peptidase substrate sequence that are required to define substrate specificity at a specified threshold for an individual sample which contains one or more known or unknown peptidases. Examples are provided for how to build pharmacologically disease-, tissue- and/or cell-selective peptidase cleavable substrates from Gearr™ motifs, and for how to incorporate these into therapeutic agents and diagnostic tools.

In some embodiments, the Alauna™ method comprises one or more component methods that have multiple routes to obtain a final peptidase substrate, depending upon the selectivity required for these substrates. In some embodiments, positional scanning techniques can be employed using the methods and libraries of the invention.

According to one aspect of the invention, the Alauna™ method comprising library screening step increases the sequence data information content.

In one embodiment, screening step comprises analyzing the population of cleaved and non-cleaved peptides or proteins.

In one embodiment, the Alauna™ method specifies that the site of cleavage within a candidate substrate sequence is identified, providing an essential position register required for data analysis.

In another embodiment, the Alauna™ method comprises identification of sets of unique candidate substrate sequences from across a full range of cleavage efficiencies, from high, intermediate, or low cleavage to the set of non-cleaved substrate sequences. It is the quantitative analysis of the rate and yield of cleavage for these sets of candidate substrate sequences against all the other unique candidate substrates within a library that enables comparison for pharmacologically selective cleavage between targeted disease- and non-diseased or healthy tissues.

In some embodiments, peptidase cleavable substrate library is a peptide or protein library containing a series of peptides or proteins of different sizes ranging from 2 amino acids to 200 amino acids. In some embodiments, the peptidase cleavable substrate library is a mixed library containing peptides or proteins of different sizes.

In one aspect, provided herein is a prodrug conjugates capable of releasing a drug upon activation by one or more peptidases.

In some embodiments, the prodrug conjugate comprises a therapeutic moiety, a masking moiety, a cleavable peptide or protein linker, optionally a carrier moiety, optionally a targeting moiety and optionally a half-life extension moiety, wherein the cleavable peptide or protein linker is peptidase cleavable substrate or disease-, tissue- and/or cell-selective peptidase cleavable substrate obtained by Alauna™ method.

In some embodiments, the masking moiety binds to the therapeutic moiety and inhibits a biological activity of the therapeutic moiety, the therapeutic moiety is optionally fused to the carrier moiety, and the masking moiety is fused to the therapeutic moiety or optionally to the carrier moiety through a cleavable peptide or protein linker.

In some embodiments, the therapeutic moiety is a cytokine moiety and the masking moiety is a cytokine antagonist.

In some embodiments, the prodrug conjugate comprises a therapeutic moiety, a carrier moiety, wherein the carrier moiety comprises a cleavable peptide or protein linker, the cleavable peptide or protein linker is peptidase cleavable substrate or disease-, tissue- and/or cell-selective peptidase cleavable substrate obtained by Alauna™ method, and the therapeutic moiety is encapsulated into the carrier moiety.

In some embodiments, the therapeutic moiety is effective as an anti-inflammatory agent, an antiallergic, an antibiotic, an anticancer agent, an antidiabetic, an antiviral agent, an antihypertensive agent, an antianginal, an anticonvulsant, an analgesic, an antiasthmatic, an antidepressant, an antidiarrheal, an anti-infective agent, an antimigraine agent, an antipsychotic agent, an antipyretic agent, an antiulcerative agent, an antithrombotic or combination thereof.

The term “therapeutic moiety” will be used in its broadest sense to include any moiety capable of providing a desired or beneficial effect on living tissue. Therapeutic moieties include, but are not limited to pharmaceutical drugs, vaccines, antibodies, proteins, peptides and nucleic acid sequences (such as supercoiled, relaxed, and linear plasmid DNA, antisense constructs, artificial chromosomes, or any other nucleic acid-based therapeutic), and any formulations thereof.

In some embodiments, the carrier moiety may be selected from lipid-based carrier systems such as anionic (conventional) liposomes, pH sensitive liposomes, immunoliposomes, fusogenic liposomes, neutral lipid nanoparticles, charged lipid nanoparticles, and charged lipid/antisense aggregates.

In some embodiments, the targeting moieties of the prodrug conjugates may be an antigen-binding moiety, or a moiety that is not an antigen-binding moiety. The targeting moieties may target to a specific healthy or disease site in the body, such as a tumor site.

In some embodiments, the half-life extension moiety may improve the pharmacokinetic profiles such as serum half-life of the prodrug conjugate.

### DETAILED DESCRIPTION OF THE DRAWINGS

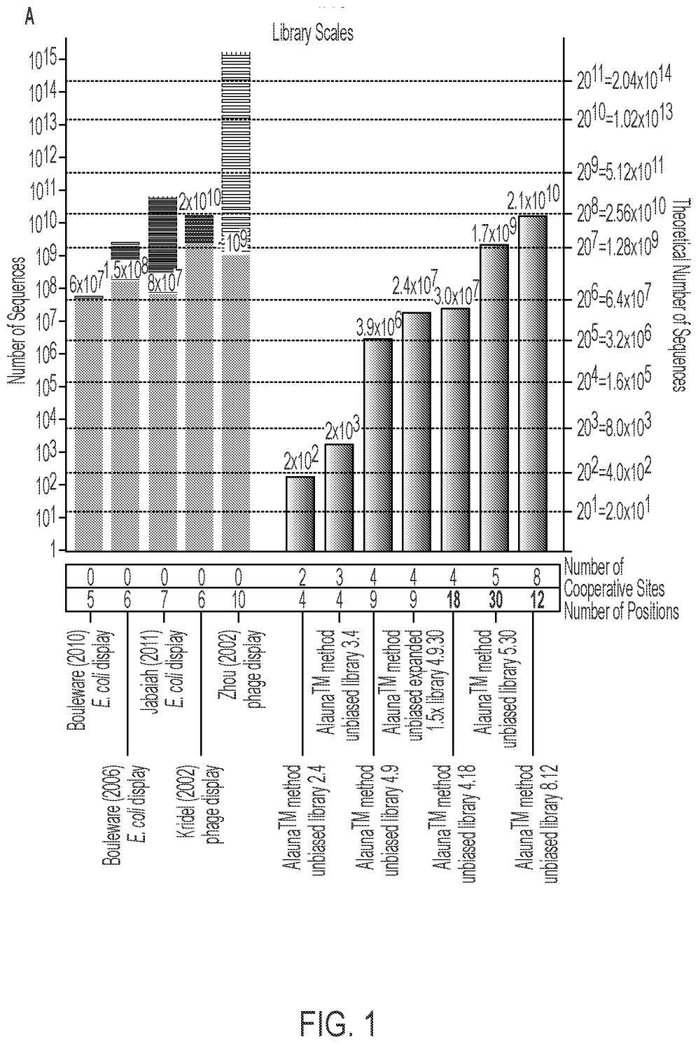

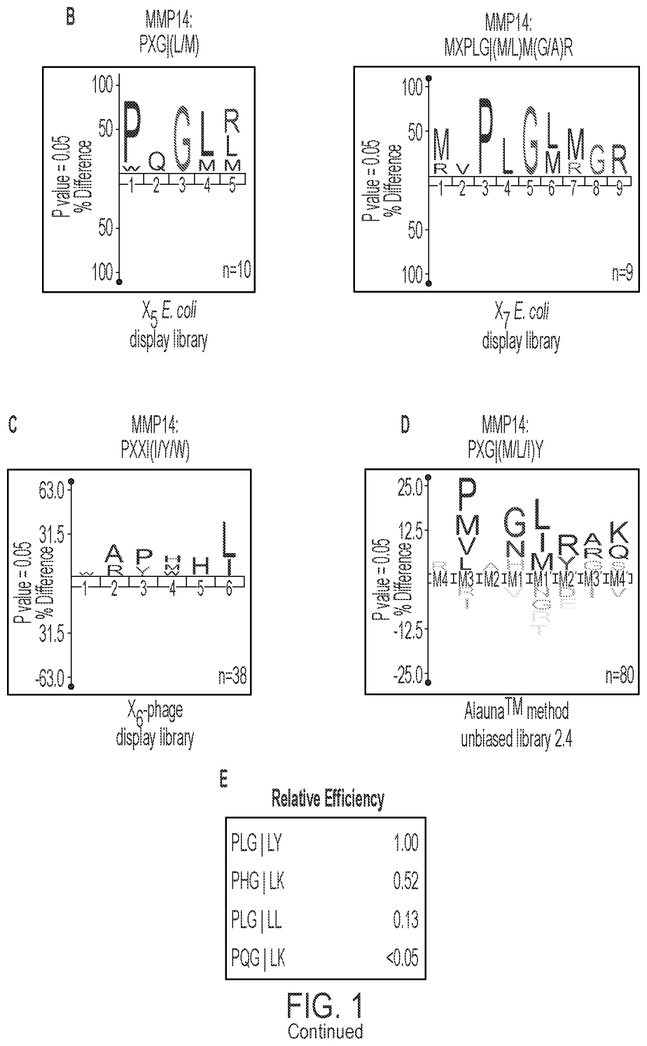

FIG. 1 depicts the library scales and sequence space coverage by Alauna™ method and existing random combinatorial library methods in the art.

The total number of unique sequences are shown for a series of libraries, comparing random combinatorial libraries with “unbiased libraries” designed using the Alauna™ method (FIG. 1, panel A). The theoretical library sizes for incorporating all possible sequence variants for the twenty natural amino acids at up to 11 sites is shown on the secondary vertical axis. Shown here are three examples of E. coli display libraries and two phage display libraries that were built using PCR with NNS or NNK degenerate codons, to produce random sequence variations of the twenty natural amino acids at S, 6, 7, or 10 sites within candidate substrate sequences. The total number of variants that were produced experimentally in each library were on the order of 10⁸ sequence variants for the E. coli libraries and 10⁹ or 10¹⁰ for the phage display libraries (shown in solid grey), consistent with the practical size limits of these two platforms. In order to overcome codon bias and inefficiencies in library generation, however, a larger number of clones are required than can practically be generated, as calculated with a Poisson distribution at the 90% confidence threshold (line pattern fill) (Clackson & Wells, 1994).

In contrast, the Alauna™ method uses efficient library design to test cooperative interactions between two or more residues within a larger number of positions than can be accessed with random combinatorial libraries. The number of cooperative interactions and the number of variable positions considered are counted below the bar graph for each library shown. Unbiased libraries have been designed on several scales with the Alauna™ method as shown (grey gradient-fill bars). The Alauna™ method unbiased library 3.4 tests 3-residue cooperativity within 4 sites, or 2-residue cooperativity between 9 sites with a library size of 2×10³ sequences. The Alauna™ method unbiased library 4.9 tests four-residue cooperativity between 9 sites with a library size of 3.9×10⁶ unique sequences. This library would accomplish the same goal as a random combinatorial library that aims to test variants within 9-sites, which would require a theoretical number of unique sequences of 20⁹=5.12×10¹¹, a scale that is inaccessible with current random combinatorial display library technology. Correcting for codon bias and inefficiencies in library generation, such a library would require 8.0×10¹³ clones: thus, the Alauna™ method unbiased library 4.9 affords a savings of production and analysis costs for 10⁷ sequences. This savings on library size can be used to test longer-range combinations of residues that could affect structural stability or other molecular functions. For example, cooperativity between 4 residues arranged in all possible configurations using the 20 natural amino acids in 18 sites can be measured with an unbiased library design of 3.0×10⁷ sequences. Cooperativity between 5 residues, testing all amino acid combinations arranged in all possible configurations arranged within 30 sites can be fully accommodated with an unbiased library size of 1.7×10⁹ sequences. Random libraries generated by PCR with NNS or NNK degenerate codons are fixed in the positions that are randomized, and therefore do not have the flexibility to test so many position variations.

Alternate amino acid alphabets may also be used for library design. A library composed of a mixture of natural and un-natural amino acids could utilize an “alphabet” of greater than 20 amino acid variants, such as 30 amino acids. Unnatural amino acids comprise diverse variants of both the backbone and sidechain groups, such as beta amino acids, homo amino acids, derivatives of proline and pyruvic acid, 3-substituted alanine derivatives, glycine derivatives, ring-substituted aromatic amino acids, O-alkylated serines that mimic methionine, N-methyl amino acids, isomers of the natural amino acids such as nor-valine or nor-leucine. D-isomeric amino acids, and others. Cooperative interactions between four residues in 9 sites with an alphabet of 30 different amino acids can be tested within a library of 2.4×10⁷ unique substrate sequences. An alternate strategy uses grouping of amino acids by physicochemical properties such as aromatic or polar character and randomly selecting a residue from within that group; using II amino acid groupings of the 20 natural amino acids, cooperativity can be tested between 8 residues within 12 sites with a library size of 2.1×10¹⁰ sequences, a scale that is achievable with phage display.

To demonstrate the performance of unbiased libraries, a comparison is provided showing the consensus sequences obtained for MMP14 with three different random combinatorial display libraries (FIG. 1, panels B and C) and the Alauna™ method (panel D). The Alauna™ method yielded n=80 cleavages with the consensus sequence PXG|(M/L/D)Y, wherein the bolded residues showed cooperative effects with each other and with additional residues in other positions, and the sequences of non-cleaved substrates were obtained, as shown in the negative region of the sequence logo graphic (panel D). As the consensus sequence from the Alauna™ method is narrowed to only describe cooperative interactions with a specific two-fold enrichment in the cleaved sequences from the library, this consensus sequence is recharacterized as a Gearr™ motif. From this experiment, the identity of favored residues at Gearr™ motif positions M₃, M₁, M₁′ and M₂′, corresponding to the bolded cooperative interacting residues, were directly identified with an efficient unbiased library of 2×10² sequences (Alauna™ method, unbiased library 2.4). In comparison, the E. coli display method required first round screening with a 5-site varied library containing 6×10⁷ unique sequences (Boulware, 2010), followed by secondary screening with a 7-site varied library containing 8×10⁷ unique sequences (Jabaiah. 2011) (panel B). The phage display library that varied 6 sites identified 38 unique sequences (Kridel, 2002) with a consensus logo as shown (panel C). The resulting sequence logos from these random combinatorial library screens showed similarities to the Alauna™ method result, but the analysis did not yield sufficient numbers of cleaved sequences (n=9, 10, or 38) to identify cooperative effects, nor were non-cleaved substrate sequences obtained. In general, those cooperative interactions that are obtained in an Alauna™ method screen can also be further explored using a second round of screening, for example using a positional scanning design, to test the effect of individual substitutions on overall cleavage. Using a kinetic analysis, the relative catalytic efficiency for four substrates incorporating residues from the four consensus sequences are shown (panel E). The top-performing sequence was the one that incorporated the MMP14 Gearr™ motif identified with the Alauna™ method. The substrate bearing a substitution drawn from the phage library consensus had 0.52-fold efficiency compared to the top substrate, and the two substrates read directly from the sequence logos from the E. coli display random library method performed at <0.05 and 0.13-fold efficiency. This demonstrates the opportunity to efficiently explore sequence space with the Alauna™ method to arrive at Gearr™ motifs.

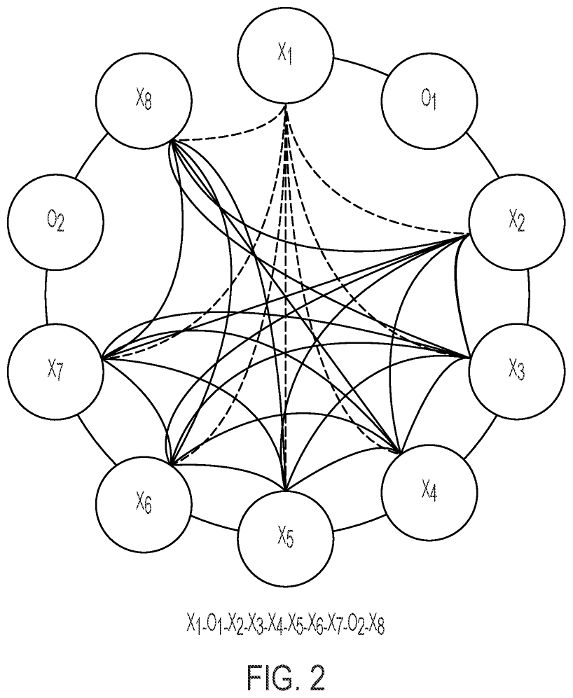

FIG. 2 depicts a substrate sequence schematic for an example unbiased library design problem. Here, the substrate sequence was required to contain ten positions, two of which were fixed positions (labeled O₁ and O₂), and eight were variable positions (labeled X: through Xx). The two fixed positions were points of derivatization for other structural features, and the goal was to place the scissile bond between O₁ and O₂, and to avoid cleavage between positions X₁ and O₁, or O₂ and X₈. In this scenario, there are twenty-eight possible cooperative interactions between each of the X positions that can be tested in a Gearr™ motif, as shown with curved dashed and solid lines. There were forty-eight possible four-position Gearr™ motif configurations that could be placed within this structure, and all unique sequence combinations of those configurations can be represented in an unbiased library design comprised of 3.9×10⁶ unique substrate sequences.

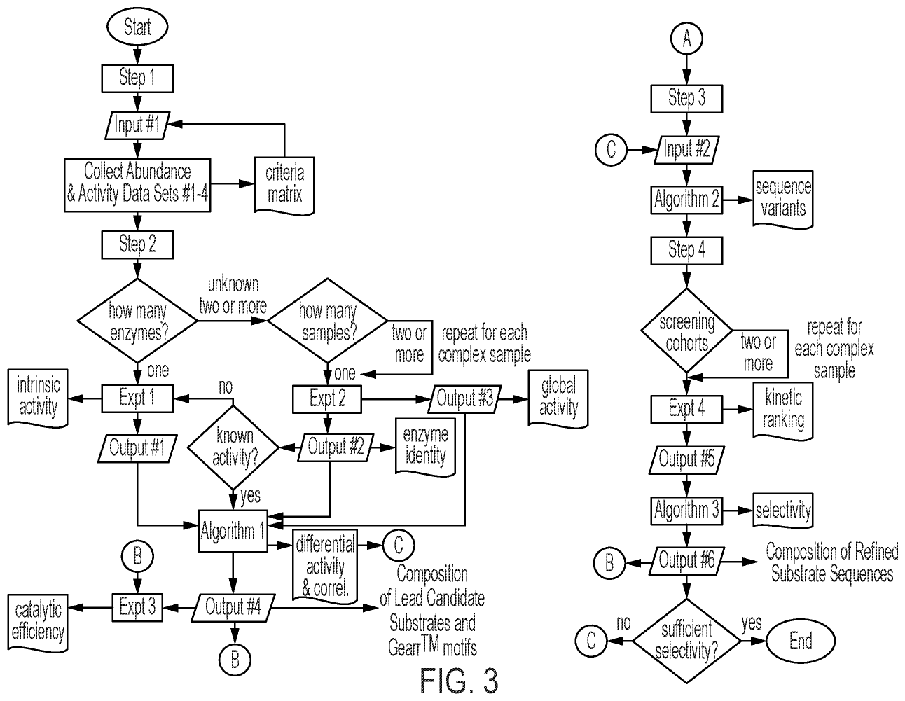

FIG. 3 depicts exemplary methods and compositions for the Alauna™ Method for identification of peptidase cleavable substrates. Broadly, this method progresses from hypothesis generation (Step 1), to discovery (Step 2), to design (Step 3) and evaluation (Step 4) of peptidase-cleavable substrates. This method provides both experimental methods for the collection of data as well as computational methods for the use of existing data to support each step in the process, and a therapeutic or diagnostic development program may choose to use only a portion of this process to develop candidate peptidase substrates.

Step 1: In Step 1, the project is designed around a therapeutic target for a disease (Input #1) that is comprised of at least one of the following: 1) a drug target such as a molecule or receptor associated with a target disease, tissue, or cell type, and or 2) a target peptidase or set of peptidases that are associated with the target disease, tissue or cell type.

Input #1: One skilled in the art can select a drug target and/or a target peptidase or set of peptidases based on their abundance and activity in the following three conditions.

Condition #1: The first condition is the site of action for the drug, which is specified by the disease status and the location of the drug target in the body, such as within a specified organ, tissue, cell type, or subcellular compartment.

Condition #2: The second condition is found in non-diseased or in healthy tissues or compartments that also express the drug target; these sites may be susceptible to undesired “on-target” “off-tissue” effects in the non-diseased or healthy tissues, where drug activity can lead to adverse events.

Condition #3: The third condition is found in healthy tissue or biological compartments that express the peptidase or set of peptidases. The third condition could also be a healthy tissue or biological compartment that expresses cognate peptidases which produce the same peptidase activities as those found in Condition #1.

Preferably, the drug target is uniquely present in Condition #1, or at least highly expressed or highly active in Condition #1 over Condition #2. It is also preferred that the activity of the targeted peptidase or set of peptidases is uniquely present in Condition ##1 and absent in Condition #2, and that it is more active in Condition #1 than in Condition #3.

In a special case, the drug target can itself be a target peptidase. In this scenario, the activity of the peptidase may reveal a pro-drug inactivator that is designed to inhibit the peptidase. The logic of the Alauna™ method described as follows still applies.

The description provided here is discussed in the context of a drug target, with the intention of pharmacological intervention with a designed molecule. However, the probe design may not be intended to have a pharmacological effect, and instead may be a reporter molecule for diagnostic probe design. The logic of the Alauna™ method described as follows still applies.

If a target peptidase or set of peptidases is specified in Input #1, then on the level of specificity and activity, it is preferred that the targeted peptidase or set of peptidases has the potential to cleave a substrate sequence with suitable rates that other peptidases will not cleave. It is also possible to work through the hypothesis generation Step 1 without knowledge of the peptidases or the peptidase activity in Conditions #1 and #2. At the outset of a drug or diagnostic development program, the Input #f1 information may be incomplete. The following process is used to collect this information.

Based on the input hypothesis (Input #1), data are collected that contain information on the expression levels of both the drug target and the targeted peptidase or the set of all peptidases, and on the activity and specificity of those peptidases, across the three Conditions specified in Input #1. Typical data sets are described below.

Data Set #1: Data Set #1 contains abundance information for the drug target and peptidases, including any targeted peptidases in Conditions #1, #2 and #3, obtained from but not limited to analyses employing the following techniques:
a) transcriptional analysis, measuring mRNA levels using techniques such as RNASeq. RT-PCR and related methods;
b) genomic analysis, measuring copy number variations that may lead to alterations in protein abundance;
c) quantitative proteomic analysis, measuring individual protein abundances with mass spectrometric detection methods; and
d) quantitative immunodetection analysis, such as enzyme-linked immunoassay (ELISA), Western blotting, antibody-protein or peptide microarrays and other methods that employ antibodies specific to the drug target or to the target peptidases to quantify these proteins.

Data Set #1 may also be obtained from one or more databases, that contain molecular abundances that were measured at genomic copy number, mRNA or protein level. This data set may be incomplete, and is either supplemented or partially replaced by experimental data obtained in Experiment #2 (Output #2)
a) Exemplary Data Set #1 contains all of the Human Protein Atlas (Uhlen et al., 2015).
b) Alternatively, Data Set #1 contains all of the data accessible at the Genomic Data Commons Data Portal, which include studies from The Cancer Genome Atlas (Gao et al., 2013; Gao et al., 2019).
c) Alternatively, Data Set #1 contains all of the European Molecular Biology Laboratory (EMBL) or European Bioinformatics Institute (EBI) gene expression atlas data (Papatheodorou et al . . . 2020).
d) Alternatively, Data Set #1 contains all of the Cancer Cell Line Encyclopedia data (Ghandi et al., 2019).

Data Set #2: Data Set #2 contains peptidase substrate information that may be measured in vitro, independent of Conditions #1, #2, or #3. Preferably, this information includes at least one substrate and multiple substrate sequences for each peptidase. This data set may be incomplete, and is either supplemented or replaced by experimental data obtained in Experiment #1 (Output #1). Data Set #2 may also be obtained from one or more databases, that contain substrate identities at either the protein level or the substrate sequence level.
a) Exemplary Data Set #2 contains all of the MEROPS database (Rawlings et al., 2016; Rawlings et al., 2004).
b) Alternatively, Data Set #2 contains all of the CutDB database (Igarashi et al, 2007).
c) Alternatively, Data Set #2 contains all of the UniProt database (Uniprot: a worldwide hub of protein knowledge. NAR 47, D506-515 (2019)).
d) Alternatively, Data Set #2 contains all of the Human Protein Reference Database (Keshava Prasad et al., 2009).

Data Set #3: Data Set #3 contains peptidase activity data and is linked to Data Set #2. One skilled in the art will obtain peptidase activity data from either in vitro and/or in vivo studies for the targeted peptidase or set of peptidases, or for closely related peptidases using peptidase activity assays and other biochemical methods, such as measurement of reporter substrate cleavage under steady-state kinetic conditions, or monitoring of the disappearance of a substrate protein in the presence of the peptidase by immunodetection. The preferred experiments characterize the following features that affect enzyme kinetics:
a) The binding affinity under equilibrium binding conditions for the peptidase toward specified substrates.
b) The rate of cleavage for a peptidolytic event either in vitro or in vivo, measuring the conversion of substrate into product for specified substrates.
c) Preferred reaction conditions for peptidase activity, measuring pH dependence, temperature, and buffer effects on the kinetics of cleavage for specified substrates.

These data may also be obtained from public databases. Exemplary Data Set #3 contains all of the BRENDA database (Jeske et al., 2019).

Data Set #4: Data Set #4 contains subcellular localization data for the drug target and target peptidases in Conditions #1, #2, and #3.

One skilled in the art will perform subcellular localization experiments using methods such as immunofluorescence to detect and/or quantify the abundance of the drug target and target peptidases within subcellular compartments using fluorescence microscopy. A target peptidase may also be detected through the use of activity probes, comprised of a peptidase substrate sequence and a reporter tag for detection, such as those probes employed for metallopeptidases, serine peptidases or cysteine peptidases (Barglow et al., 2007; Greenbaum et al., 2000; Sieber et al., 2006; Kidd et al, 2001).

Alternatively, bioinformatic annotations and information from public databases may be used to approximate subcellular localization.
a) Exemplary Data Set #4 contains all of the subcellular localization data from the Human Protein Atlas (Uhlen et al., 2015).
b) Alternatively, Data Set #4 contains all of the UniProt database. (Uniprot: a worldwide hub of protein knowledge. NAR 47, D506-515 (2019)).
c) Alternatively, Data Set #4 contains all Gene Ontology annotations for subcellular localization (The Gene Ontology Resource: 20 years and still Going strong. NAR 47, D330-D338 (2019)).

Information from Data Sets #1 through #4 is extracted, processed, and scored as follows:

Data Set #1 is filtered to collect the abundance information for the drug target and all known peptidases, across the three Conditions specified in Input #1. A correlation analysis is performed to identify peptidases that are most highly co-expressed with the drug target at the site of action in Condition #1 as compared to Conditions #2 and #3. An enrichment ratio is also calculated for the abundance of the drug target and for all peptidases, calculating enrichment in Condition #1 as compared to Conditions #2 and #3. The larger the differential ratios in each case, the higher the enrichment score for this feature.

Data Set #2 contains all sequences known to be cleaved by all peptidases. For each peptidase, the first step of analysis is to align the set of substrates identified for that peptidase in Data Set #2 by their scissile bond and to determine the amino acid frequency at each position in the substrate sequence. A score for ranking the probability of specificity of a peptidase compared with other peptidases is then calculated as a weighted product of the individual site frequencies for the highest frequency amino acid in each position within the analyzed substrate sequences. These calculations are negatively affected by biased profiling methods; thus, it is preferred that these data are generated by the Alauna™ method with unbiased libraries (Experiment 1, Output #1). Peptidases that accept only a limited number of amino acids in two or more positions within their substrates score with higher specificity probability, and these peptidases are preferred as target peptidases because this indicates a higher probability that a Gearr™ motif can be produced for these peptidases with the Alauna™ method.

Data Set #3 is a collection of enzyme kinetics terms, which may include binding affinity for specified substrates, catalytic rate, or catalytic efficiency, that are preferably measured under experimental conditions that closely approximate Conditions #1, #2 and #3 in Input #1. These experimental kinetic data are filtered to focus on the activity of each peptidase toward the substrate sequences that have the highest catalytic rate or catalytic efficiency for each peptidase. Each peptidase is then given a score that is a median-normalized numerical value for its maximum catalytic activity, that can be used to rank the peptidases against each other.

A corollary to the analysis of Data Set #3 considers the special case that the target application may require amino- or carboxypeptidase activity versus endopeptidase activity. In this case, Data Set #3 is weighted by the tolerance of a peptidase for accepting substrates that do not fill one or more of the N-terminal or C-terminal sites relative to the scissile bond within a substrate motif.

Data Set #4 contains subcellular localization data for all peptidases as well as for the drug target. The level of co-localization for the drug target with all peptidases can be calculated as a simple correlation when using bioinformatics information such as GO terms, but the preferred scoring is performed from quantitative imaging data. The correlation coefficient for colocalization of drug target with each peptidase signal, measured at the protein or peptidase activity level, is then calculated for each Condition identified in Input #1. A selective co-localization score is then calculated as the ratio of the correlation coefficients for colocalization measured in Condition #1 vs Conditions #2 or #3. Peptidases with the greatest differential values in the selective co-localization score are preferred.

Criteria Matrix: The report out from Step 1 is a Criteria Matrix (CM), that is used to evaluate and revise the input hypothesis (Input #1), and to select a target peptidase or set of peptidases that are hypothesized to meet a specified level of disease or tissue-selectivity in the context of peptidase-cleavable substrate design. The CM contains one or more scored features calculated from one or more of the above experimental Data Sets #1 through #4. These features include: co-expression of the target peptidase with the drug target, peptidase specificity probability score and the number of sequence positions that contribute to this score, maximum catalytic activity, and specific co-localization of the target peptidase with the drug target. The CM identifies preferred target peptidases that meet the refined hypothesis statements in Input #1 as follows.

A peptidase or set of peptidases present in a diseased tissue (Condition #1) has expression levels that correlate with the drug target. The preferred target peptidases also have enriched abundance in the targeted diseased tissue (Condition #1) over non-diseased or healthy tissues (conditions #2 and #3).

The substrate specificity of the selected peptidases is predominately defined by two or more residues, and the specificity of the selected peptidase is different from that of other peptidases present in the background (Condition #2 or #3), thus conferring greater specificity for that peptidase.

The catalytic efficiency for a targeted peptidase or set of peptidases, is predicted to be sufficient to produce a specified rate of peptidolytic cleavage.

The peptidase is co-localized within subcellular compartments with the drug target in the target tissue, and preferably the peptidase is not co-localized with the drug target in non-diseased tissues or compartments that may also express the drug target (Condition #2).

The CM also assists sample selection for Step 2 discovery experiments; these samples are specified by disease status in the patient, and features such as tissue or compartment and cell type. The CM further assists sample selection for Step 2 discovery experiments, by specifying that experimental preparations from Conditions #1-3 must accurately sample the peptidases and their activity for the screening process.

Step 2. In a rational approach, a target peptidase or set of peptidases is identified from analysis of the Data Sets #1-4 with the CM. Selection of target peptidases also affects the sample selection with the CM for Step 2 discovery experiments.

Experiment 1 is a peptidase activity assay performed under controlled reaction conditions using a pool of defined candidate substrates, with the goal of identifying cleaved substrates, the site of cleavage, and their overall rate of cleavage.

The preferred Experiment 1 is performed using a Multiplexed Substrate Profiling by Mass Spectrometry (MSP-MS) method (O'Donoghue et al., 2012). This method employs a defined library of synthetic peptides as substrates, preferably using an Alauna™ method unbiased library, designed to test cooperative interactions at a specified level, with mass spectrometric detection for identification of cleaved peptides from kinetic samples drawn from the reaction over the time of the experiment. The Output #1 of this experiment contains the site of cleavage at the scissile bond, and the rate of cleavage for all of the peptide substrates in the library that were cleaved. When this experiment is performed in vitro, the output is considered “intrinsic activity” representing the behavior of a peptidase toward peptide substrates under idealized conditions.

Alternatively, Experiment 1 is performed using an unbiased library tested in the context of protein substrates, wherein a constant protein scaffold is used to support the substrate sequences provided by the Alauna™ method unbiased library design. This protein substrate scaffold can be compatible with a biological display system such as phage display or E. coli display. In this scenario, substrate sequences are identified by next generation sequencing (NGS) or other nucleotide sequencing methods for the identification of unique clones that were cleaved in the biological display experiment, and the scissile bond is identified using mass spectrometry-based peptide sequencing. Alternatively, the protein scaffold may also be compatible with display on an artificial surface such as a nanoparticle or other synthetic construct.

Experiment 2 is performed in the case that a sample comprises two or more known or unknown peptidases. Experiment 2 is performed in two parallel steps.

Peptidase identification is preferably performed using mass spectrometry-based peptide sequencing workflow used in proteomic analysis. One skilled in the art will select a proteomic sequencing method, such as using trypsin-digestion to generate peptides, and sequencing the peptides using tandem liquid chromatography mass spectrometry (LC-MS/MS). The sensitivity of this method for peptidase detection may be further enhanced with biochemical enrichment methods such as employing activity-based generic probes that covalently label serine, cysteine and metallo-peptidases to enrich the sample for peptidases prior to protein identification (Barglow et al., 2007). The main requirement is for unambiguous identification of a protein according to generally accepted proteomic data analysis guidelines, typically performed at 1% false discovery rate. The Output #2 of this step is semi-quantitative peptidase identification in a complex sample.

If an enzyme identified in Output #2 has not yet been characterized for its intrinsic activity, that enzyme can be produced biochemically using cell based recombinant expression systems or in vitro translation, and subjected to Experiment 1.

Peptidase activity profiling of a complex sample is preferably performed using an Alauna™ method unbiased library, tested in a peptide format with multiplexed substrate profiling, or in a scaffolded protein format with a biological display system, as described in Experiment 1. In Experiment 2, the Output #3 of this step is the identification of cleaved and non-cleaved sequences within the unbiased library, along with the sites of cleavage, that are produced by two or more peptidases in the context of a complex sample. Therefore, the Output #3 of this step is called “global activity” for a complex sample.

Preferably, Experiment 2 is performed using biologically-derived samples containing active peptidases, such as conditioned cell culture media produced from the culture of cells or ex vivo tissues, or biofluids obtained directly from patients or animal models. The buffer conditions for Experiments 1 & 2 in Step 2 are preferably selected to match the conditions defined in Input #1, and identical reaction conditions (as defined by temperature, pH, and buffer composition) are used throughout Experiments 1 & 2, to assure comparability between these experiments.

To identify global signatures that can be exploited for selective peptidase cleavable substrate design, Experiment 2 is performed for multiple samples that represent the Conditions #1, #2, and #3 that were defined in Input #1 during Step 1. These samples are either primary samples from patient donors such as diseased tissues, non-diseased or “normal” adjacent tissues, healthy tissues, liquid biopsies or biofluid samples, or cell culture or tissue models for these samples. The number of samples to be used for each Condition (#1, #2 or #3) are defined through biostatistical power analysis, which depends on sample-to-sample variability within each cohort. Data sets are preferably collected from a minimum of three but ideally five or more biological replicate samples from each Condition (#1, #2 or #3).

Data from Output #1 and Output #3 are similar, and preferably contain the following information: i) identification of both cleaved and non-cleaved substrates in the library, ii) the site of the scissile bond for cleaved substrates, as well as iii) the rate of cleavage at those bonds.

Algorithm 1 is used to analyze the peptidase activity profiling data from Output #3, to perform a differential analysis of this activity profiling data between Conditions #1, #2 and #3, and to identify selective Gearr™ motifs that are preferentially cleaved in Condition #1 over Conditions #2 and #3.

Optionally, Algorithm 1 can also be used for comparative analysis between the data from Outputs #1 and #3 to parse the observed cleavages in Output #3 and assign them to peptidases identified in Output #2.

Algorithm 1 includes two main analysis steps for Output #3 peptidase activity profile data. First, the set of all candidate cleavable motifs are identified by aligning all cleaved substrate sequences within a set of cleaved substrates by their scissile bond, measuring amino acid frequencies at each position, and then measuring frequency of cooperative residues between two or more sites within the substrate sequences, which thereby define Gearr™ motifs. The user can also specify two optional filters for the sets of cleaved substrates used for Gearr™ motif analysis. Subsets of substrates can be optionally grouped by their rate of appearance in the peptidase reaction into “high” “medium” and “low” efficiency cleavage groups for Gearr™ motif identification in the first step. An optional reproducibility filter can also be applied to select the reproducible subset of cleavages within biological replicate samples from individual Conditions at a specified threshold. The second processing step employs a statistical method selected from P-value statistics or Z-score statistics to compare between samples in Conditions #1-#3, and to rank the identified Gearr™ motifs for enrichment in Condition #1 over Conditions #2 and #3.

Algorithm 1 can also be used for the comparative analysis of the data from Output #2 for samples from Conditions #1, #2 and #3. Enrichment of peptidases is assessed using a statistical method selected from P-value statistics or Z-score statistics. The input values for these statistics is the abundance of the peptidases, that can be measured within a mass spectrometric experiment such as by spectral counting. A correlation analysis can be further performed for the enrichment of specific cleavages and Gearr™ motifs with the enrichment of specific peptidases at their protein abundance level in Condition #1 versus Condition #2 and/or Condition #3.

The basic data processing output report from of Algorithm 1 is the differential activity measured with peptidase samples towards individual substrates within the unbiased library, and this data can be presented as a heat map of substrates versus their cleavage efficiencies across all samples tested in Experiment 2.

Output #4 is a set of lead candidate substrate sequences that can be refined in Step 3. These sequences are novel sequences bearing selective Gearr™ motifs that were identified from comparative analysis of selective cleavages in Condition #1 versus Conditions #2 and #3. These sequences can also include selected peptides from the Alauna™ method unbiased library, which meet a specified threshold for selective cleavage efficiency or rate in Condition #1 versus Conditions #2 and #3.

To validate the lead substrates bearing selective Gearr™ motifs from Output #4, an activity assay is used in Experiment 3 to test individual lead substrate sequences for their cleavage efficiency. Experiment 3 may be performed as a peptide-based assay using spectroscopic methods or mass spectrometric methods, or as a scaffolded-protein based assay such as using a bacterial or phage display system. The peptidase samples selected for Experiment 3 should match those that are intended for validation experiments in Step 3, and may include complex samples as were used in Experiment 2, and/or recombinant enzymes used in Experiment 1. Lead substrate sequences that are confirmed to be cleaved are then advanced as lead candidate substrate sequences into Step 3 for sequence refinement. Lead substrate sequences that are found to be non-cleaved represent non-cleavable negative control sequences, which are useful for demonstrating specific activation of a pro-drug or diagnostic probe in biochemical and in vivo validation experiments later in the design process.

Data from Experiment 3 are analyzed with steady state enzyme kinetics. Regardless of whether the platform is a synthetic peptide or scaffold protein platform, the abundance of cleaved product is measured at each time point in the assay, for example using mass spectrometric of fluorescence detection. If product formation reaches saturation, meaning a plateau can be identified in the plot of product abundance measured over time, the data can be fitted to the first order kinetics equation:

$$Y = {e}^{( - {k}_{obs} \cdot t )}$$

where Y=percent product formation, k_{obs} is the rate, and t=time. When Experiment 3 is performed with a purified enzyme, Michaelis-Menten kinetic analysis can be applied and the observed rate k_{obs}, is a function of the enzyme concentration, with [E₀] in molarity units measured at time zero, and an observed catalytic efficiency (k_{cat}/K_{M}) in units of per molarity per unit time with the equation:

$${k}_{obs} = \frac{{k}_{cat}}{{K}_{M}} \cdot [ {E}_{0} ]$$

The lead substrate sequences from Output #4 can then be compared by their relative catalytic efficiencies. Those substrates that perform with the highest differential catalytic efficiencies in Condition #1 versus Conditions #2 and #3 are selected for refinement in Step 3.

Step 3: Step 3 is a candidate substrate sequence refinement process, wherein variants of the lead substrate sequences in Output #4 are designed into a new candidate-based library that will be screened in Step 4 for performance at a specified threshold for selective cleavage in Condition #1 vs Condition #2 and Condition #3.

Algorithm 1 was used to perform a differential activity analysis for cleavages in Condition #1 vs Condition #2 and/or Condition #3, which is reported in the form of a table containing amino acid frequencies that were enriched at each position. Algorithm 1 is also used to identify cooperative interactions between two or more amino acids that are selectively enriched in the cleaved substrate sequences from in Condition #1 vs Condition #2 and/or Condition #3. This data is used as Input #2 for Algorithm 2, and serves as a substitution table for the design of sequence variants.

Additional information may be included in the new library design at the Input #2 step. For example, if the abundances and activity of two or more peptidases are found to be positively correlated with defined selectivity requirements in Algorithm 1, and are enriched in Condition #1 over Conditions #2 and #3, a new substrate sequence design may be specified to include either a hybrid motif that is favorably cleaved by these two or more peptidases, or to allow for multiple motifs to be included in tandem within a peptidase substrate sequence. Alternatively, feedback from experiments employing a protein scaffold for library production may provide insights on protein stability that require changes to the scaffold design or to bracketing residues outside of the intended cleavable substrate sequence region for improved stability. Variations include altered substrate sequence length, restricted amino acid composition, or the addition of bracketing residues outside of the intended substrate sequence region. These and other considerations may be included in Input #2 toward the design process of the cleavable substrate sequence library.

In Algorithm 2, the set of Gearr™ motifs from Output #4 are used as seed sequences that are first arranged in all possible permutations, with varied numbers of bracketing residues to adjust for spacing, within a specified substrate sequence length, and then each motif is systematically varied at each position using the substitution matrix resulting from Algorithm 1 to identify amino acid substitutions that will enhance for selective cleavage in Condition #1 over Conditions #2 and #3.

The Output from Algorithm 2 is a refined library of sequence variants that are then subjected to an activity assay in Experiment 4, performed under identical reaction conditions as were used in Experiment 2. Previously generated, as well as newly prepared peptidase-containing complex samples from Conditions #1, #2, and #3 are included in this experiment. The number of patient-derived samples used in each screening cohort for Experiment 4 will be determined by a power calculation from the patient-to-patient variance within the results of Experiment 2.

Output #5 from Experiment 4 is a kinetic ranking of cleaved substrate sequences, therefore sufficient time points are required to model enzyme progress curves for product formation. As in Experiment 2, the platform for the assay can be a synthetic peptide-based assay with mass spectrometric detection, or the library can be displayed with a scaffolded protein platform with flow cytometric sorting of cleaved clones followed by NGS sequencing to identify those clones. A kinetic experiment is performed using either platform by removing aliquots from the peptidase reaction at multiple time points and quenching the reaction, followed by sequencing of the cleaved products. In this way, the Step 3 library sequences are evaluated for the subsets that are most efficiently, or most rapidly cleaved.

The Output #5 from Experiment 4 provides for the kinetic ranking of individual cleavable substrate sequences. By testing additional sequence variants in the Step 3 library as compared to the Step 2 library, the Output #5 data can provide additional specificity information about the amino acid preferences at additional positions within a Gearr™ motif, leading to refinement. An additional outcome includes revealing the tolerance for substitution at interior positions within a Gearr™ motif that can be exploited for integration with other Gearr™ motifs. The Output #5 data also provide structural and cleavage susceptibility insights for bracketing residues outside of the intended substrate sequence regions, within the context of a larger polypeptide construct. This information is valuable for integration of the cleavable substrate sequence into a therapeutic construct or diagnostic probe design. This analysis will also reveal substrate sequence variants that are not tolerated for Condition #1 cleavage, or that abolish selective cleavage between Conditions #1, #2, and #3.

Algorithm 3 is used to evaluate the selective cleavage of each substrate sequence within the Step 3 library for enhanced cleavage in Condition #1 versus Conditions #2 and #3 as defined in Input #1. Enrichment of a specific cleavage Gearr™ motif is again evaluated using a statistical method selected from P-value statistics or Z-score statistics, in Algorithm 3. The input values for Algorithm 3 are the observed rate of cleavage, k_{obs}, for each substrate. The substrates with the largest fold-differences in the observed rate of cleavage between Condition #1 and Condition #2 and/or Condition #3 are the most selective for Condition #1. Output #6 consists of the refined set of peptidase substrate sequences, containing one or more identified Gearr™ motifs, along with their measured selectivity, which is output as a ratio of the observed cleavage rates for Condition #1 vs Condition #12 and Condition #3.

These novel linker sequences containing one or more Gearr™ motifs represent refined selective substrate sequences that can then be incorporated into therapeutic molecule and reporter substrate design. The individual catalytic activity for each peptidase motif is validated in Experiment 3, using purified or recombinant candidate peptidases identified in Output #2.

In the last step of the process, a candidate substrate sequence is designed into its intended therapeutic construct or diagnostic probe design, and evaluated for whether or not it has sufficient selectivity for Condition #1 versus Conditions #2 and #3 to meet the specifications in Input #1. If the selective cleavable substrate sequence has met the specifications for selective cleavage in the conditions defined in Input #1, the process is complete.

If selectivity is insufficient, the process of Step 3 library design is repeated. A new Input #2 hypothesis is developed that takes into consideration the results of Experiment 4 to develop a new design. Parameters that may be considered are the initial placement of peptidase motifs within a linker design, and the domain architecture of the design target molecule.

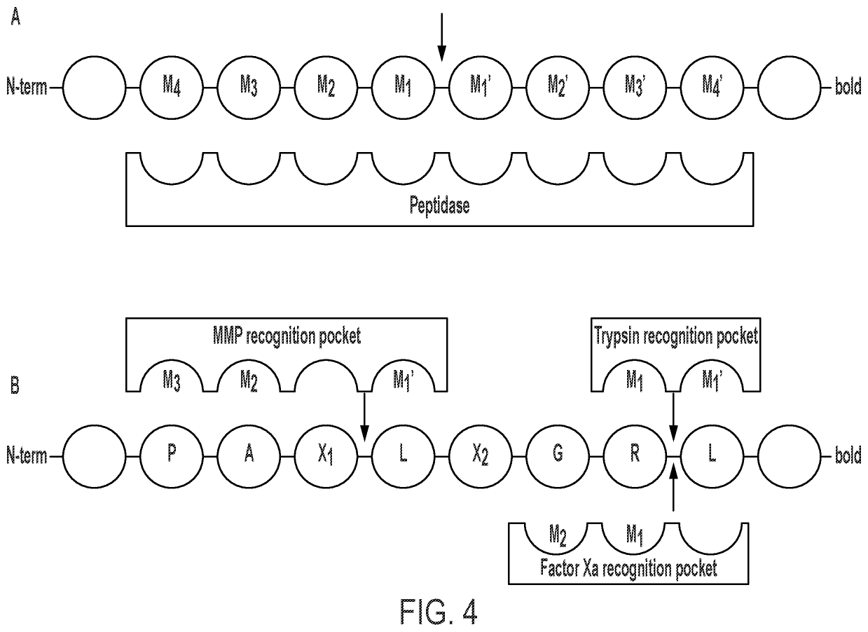

FIG. 4 depicts (A) Gearr™ motif nomenclature and (B) a substrate sequence diagram that demonstrates how two or more Gearr™ motifs can be incorporated into a candidate substrate sequence. (A) Gearr™ motif positions are numbered with index numbers that increase with distance from the scissile bond. In the N-terminal direction, the Gearr™ motif positions are numbered M₁, M₂, M₃, M₄ etc., and in the C-terminal direction, the Gearr™ motif positions are numbered M₁′, M₂′, M₃′, M₄′ etc.; thus, the scissile bond for a peptidase is between motif positions M₁ and M₁′ as indicated with an arrow. (B) Shown are Gearr™ motifs for three human peptidases, incorporated into a candidate substrate sequence. Human trypsin has a Gearr™ motif that is mathematically defined by residues in motif positions M₁ and M₁″, in that 99% of its known substrate sequences contain Arg or Lys residues in position M₁ and <1% contain Pro in M₁′. This is contrasted with the generally accepted consensus motif for trypsin that only reports on the positive cleavage of substrates bearing Arg or Lys residues N-terminal to the scissile bond. The Gearr™ motif for Factor Xa was defined by residues in motif positions M₂ and M₁, preferring Gly (48% frequency) in position M₂, and Arg (87% frequency) in M₁. A Gear™ motif for MMP2 is shown that is dependent upon positions M₃, M₂ and M₁′, preferring Pro (at 24% frequency), Ala (at 19% frequency) and Leu (at 41% frequency) in these positions, respectively. Thus, a peptidase may have an extended substrate recognition pocket (cartooned as a grey recognition block) that can interact with the substrate sequence, but the Gearr™ motif is given by a subset of recognition sites that describe the specificity of the peptidase at a mathematically defined threshold and that by definition, arise from cooperative interactions.

The example substrate sequence PAX₁LX₂GRL is shown that incorporates the three Gearr™ motifs for MMP2, trypsin, and Factor Xa. Factor Xa and trypsin have compatible Gearr™ motifs that result in cleavage at the same scissile bond in this substrate sequence, and the combination of their overlapping motifs produces a hybrid Gearr™ motif. The MMP2 Gearr™ motif is not compatible with these enzymes, and therefore it was placed at an adjacent site, creating a tandem substrate containing two scissile bonds. Position X₁ represents a single variable residue site because it is within the MMP2 Gearr™ motif, and position X₂ represents a variable bracketing residue position that can contain multiple amino acid insertions.

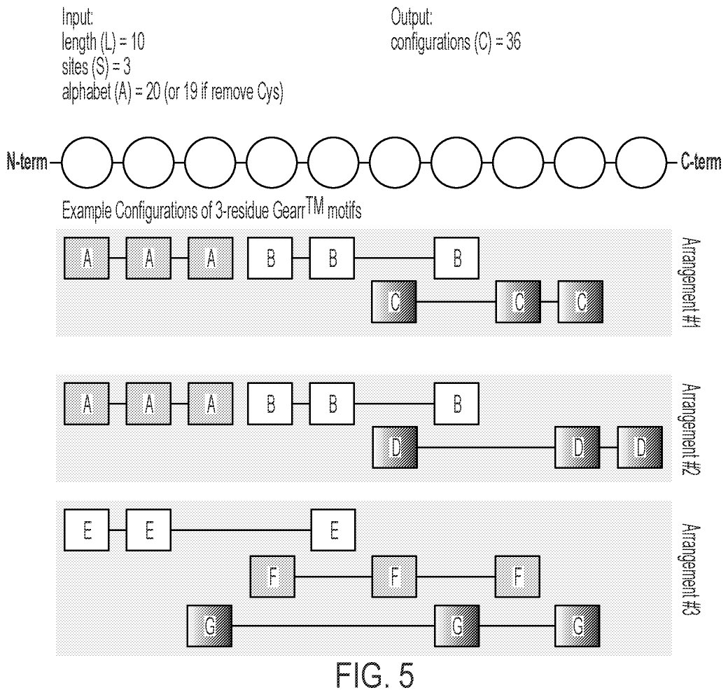

FIG. 5 depicts the input features for the Alauna™ method unbiased library design. Input parameters include the length (L), the number of sites(S), and the alphabet of allowed amino acids (A) that specify a Gearr™ motif. The unbiased library is then designed to include all possible Gearr™ motifs at the level specified by the (L, S and A) terms. In this example, the substrate sequence limits the length of a Gearr™ motif, and there are 36 possible configurations consisting of 3 sites within a maximum length of 10 residues; these 36 configurations can be placed into substrate sequences with a minimum of 16 different arrangements, examples of which are shown. A unique Gearr™ motif is comprised of a unique configuration and the identity of the amino acids that are placed within that configuration. The Alauna™ method requires that an unbiased library should comprise all of the unique Gearr™ motifs specified with L. S and A input values. The Alauna™ method unbiased library 3.10 using the 20 natural amino acids and containing 1.28×10⁵ total unique sequences assures that all unique three-position motifs comprising cooperative interactions within the length of ten positions are represented in this library at least once. After screening with a peptidase assay, the cleaved substrate sequences are analyzed with Algorithm 1 (FIG. 3) to identify the motifs that are enriched from the library as Gearr™ motifs for those peptidases.

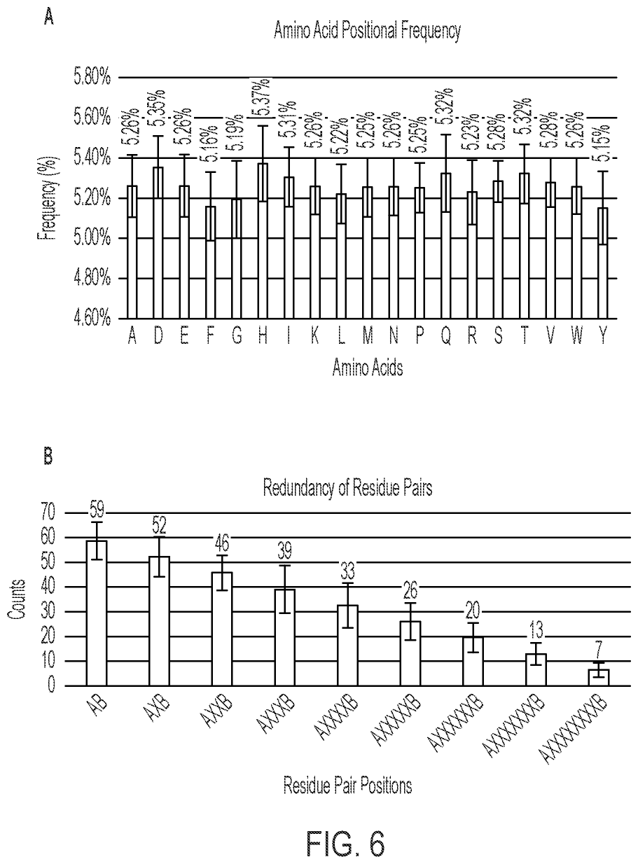

FIG. 6 depicts the amino acid positional frequency (A) and redundancy (B) for the Alauna™ method unbiased library 3.4. The library design parameters were L=4, S=3, and A=19 (using the twenty natural amino acids and excluding Cys to avoid oxidation) to enumerate all the possible Gearr™ motifs at this library scale. These were allowed to be placed in all configurations within a substrate sequence of 10 positions, and resulted in a library size of 2.3×10³ unique sequences. The amino acid composition in the overall library was on average 5.26% for each amino acid (equivalent to 1/19), and the frequency of each amino acid's appearance at each position was also evenly distributed across the substrate sequence. All three-position combinations of 19 amino acids within 4 motif positions were also represented at least once in the library, with repeat appearances, defined here as redundancy, ranging from 1-3 appearances. (A) The average frequency that each amino acid was found at each of 10 positions within the substrate sequences is shown. (B) The average redundancy is shown for all possible cooperative interactions between residues in positions that are directly adjacent to each other (denoted AB) or that are separated by one to eight random amino acids (X).

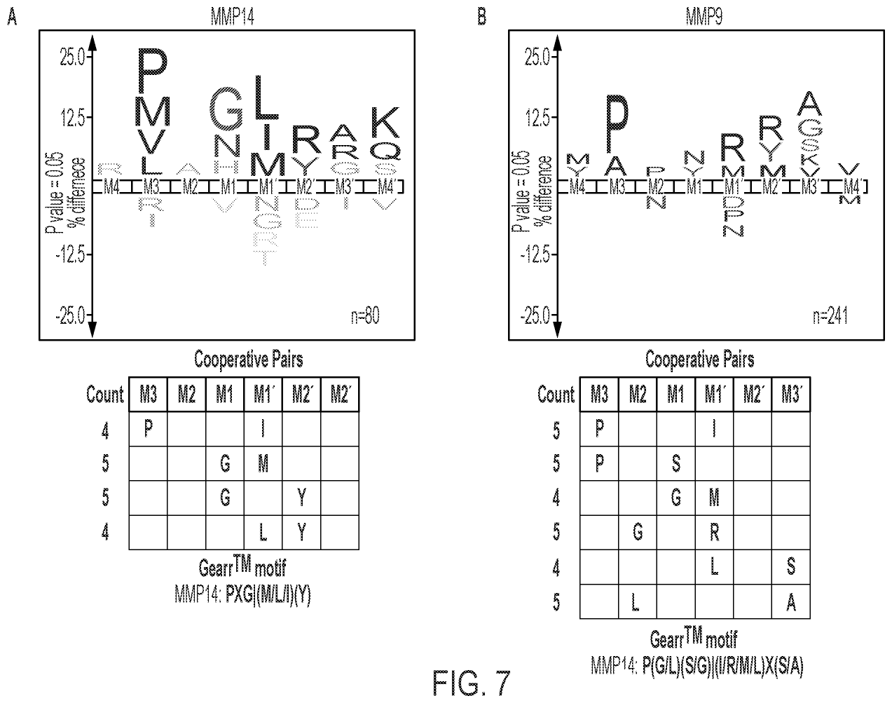

FIG. 7 depicts sequence logos generated for MMP14 (A) and MMP9 (B) as the results of an Experiment 1 (FIG. 3) peptidase profiling activity assay using the Alauna™ method unbiased library 2.4. The icelogo program v 1.2 was used to generate a sequence logo that visualizes amino acid frequencies observed among cleaved substrates, as compared to a background model defined by the complete library and shown as a percent difference at p=0.05. The Alauna™ method unbiased library 2.4 in this case was built with synthetic peptide substrates, with a library design input of L=4, S=2, and A=19 (excluding Cys to avoid oxidation) within a substrate sequence that was limited to 10 amino acids, which resulted in a library that represents all combinations of amino acid pairs at least twice within the library. The more frequently observed pairs are considered cooperative sites that comprise a Gearr™ motif. (A) Among the n=80 cleavages observed for MMP14 in this library, there were four cooperative pairs of amino acids that were observed 4 or 5 times. The co-association of these cooperative interactions result in a Gearr™ motif of PXG|(M/L/I)Y for MMP14, indicating that Gearr™ motif positions M₃, M₁, M₁′ and M₂′ and the amino acids within those positions control substrate specificity for MMP14. (B) Analysis of MMP9 using the same method resulted in the Gearr™ motif P(G/L)(S/G)|(I/R/M/L)X(S/A) for MMP9, indicating that Gearr™ motif positions M₃, M₂, M₁, M₁′ and M₃′ control the specificity for MMP9.

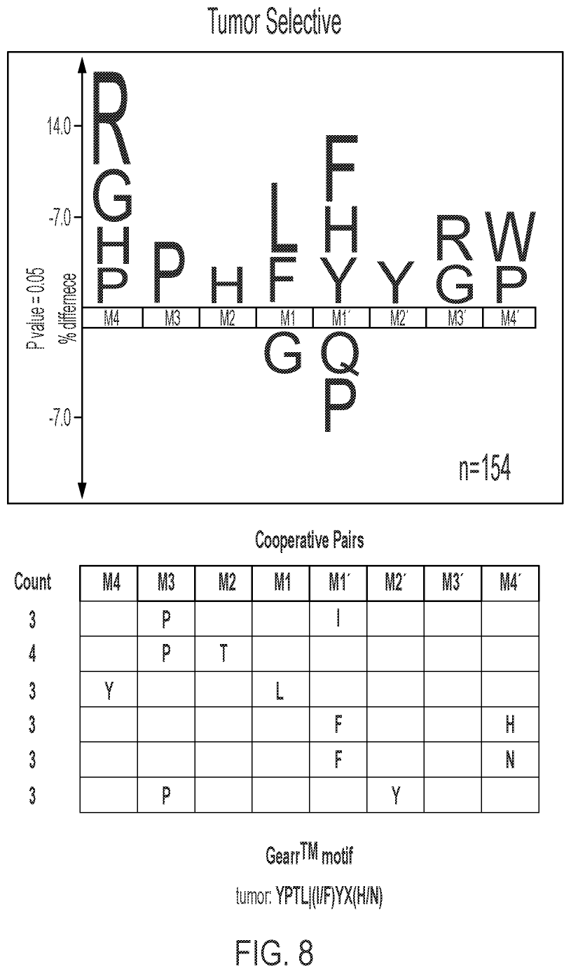

FIG. 8 depicts the sequence logo generated for tumor-selective cleavages identified from conditioned media samples that were prepared from surgically resected papillary thyroid carcinoma and matched non-diseased tissue. These data were output from a peptidase profiling activity assay in Experiment 2 (FIG. 3, Output #3) using the Alauna™ method unbiased library 2.4. Data were analyzed as in Experiment 1, with the exception of adding an additional step that this selected set of cleavages represent those uniquely observed in the tumor sample and not observed in matched non-diseased tissue. The icelogo program v 1.2 was used to generate a sequence logo that visualizes amino acid frequencies observed among cleaved substrates, as compared to a background model defined by the complete library, and shown as a percent difference at p=0.05. Among the n=154 tumor-selective cleavages observed in this library, there were six cooperative pairs of amino acids that were enriched above background and observed 3 or 4 times, including two consistent with the MMP14 Gearr™ motif (FIG. 7, panel A). The co-association of these cooperative interactions result in a tumor-selective Gearr™ motif of YPTL|(1/F)YX(H/N), indicating that Gearr™ motif positions M₄, M₃, M₂, M₁, M₁′ M₂′ and M₄′, and the amino acids within those positions, control tumor selectivity in these papillary thyroid cancer samples.

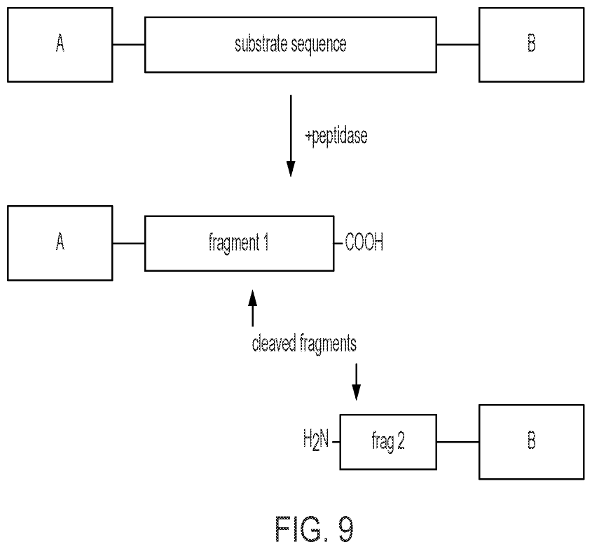

FIG. 9 depicts a diagram that includes a substrate sequence.

### DETAILED DESCRIPTION

### 1. Definitions

All references cited herein are incorporated by reference in their entirety as though fully set forth.

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeck, David H Margulies, John Wiley and Sons, Inc., (2003); each of which provide one skilled in the art with a general guide to many of the terms used in the present application.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC. The degenerate DNA codons described as NNN, NNS, or NNK, when included in the synthesis of an oligonucleotide, indicate that any of the four bases C, G, A, or T can be inserted into the position “N”, C or G can be inserted for “S”, or G or T can be inserted for “K”.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

As used herein, the term “cooperativity” refers to the identity of the amino acids at additional sites elsewhere in the substrate sequence that influence overall substrate cleavability by the peptidase. Cooperativity is lost or ignored when the rate of co-occurrence between amino acids in various positions is not considered.

As used herein, the terms “polypeptide” and “protein” used throughout synonymously refer to any polymer formed from multiple amino acids associated, at least in part, by covalent bonding (e.g., “protein” as used herein refers both to linear polymers (chains) of amino acids associated by peptide bonds as well as proteins exhibiting secondary, tertiary, or quaternary structure, which can include other forms of intramolecular and intermolecular association, such as hydrogen and van der Waals bonds, within or between peptide chain(s)), unless otherwise stated. The term “polypeptide” and “protein” includes fusion proteins, unless otherwise stated. A fusion protein can be any polypeptide comprising multiple (e.g., two or more) distinct polypeptides, regardless of their relative location within the polypeptide.

As used herein, the term “peptidase” means an enzyme that is capable of cleaving a peptide bond within a polypeptide; such peptide bond and at least one adjacent amino acid, or the peptide sequence containing such peptide bond, is termed the “substrate sequence”. The polypeptide containing the peptide bond is the “peptidase substrate”. The peptidase substrate may contain one or more polypeptide polymers, and may contain one or more non-polypeptide moieties. Such polypeptide polymers and non-polypeptide moieties may be linked covalently or non-covalently. As used herein, the cleavable peptide bond is termed the “scissile bond” in the peptidase substrate.

Peptidases can be described as “exo-peptidases” or “endo-peptidases”. Exo-peptidases cleave at the N- or C-terminus of a polypeptide chain and include “amino-peptidases”, which cleave the peptide bond after one, two, or three amino acid residues from the N-terminus, and “carboxy-peptidases”, which generally cleave a single amino acid residue from the C-terminus. Endo-peptidases cleave at sites more distant from the N- or C-termini of a polypeptide. Omega-peptidases are distinct from endo-peptidases and cleave near N- or C-termini, but they do not require recognition of the charged amino or carboxyl groups at the termini.

The standard nomenclature to describe peptidase assays follows biochemical definitions. Peptidase assays monitor the cleavage of “substrate” and the formation of “product”, and can be monitored at a single time point, called an “end point assay”, or at multiple time points over the course of a reaction using a “continuous assay”. An end point assay is used to measure the yield of product formation or the disappearance of substrate, and a continuous assay is used to determine rates for substrate cleavage or product formation.

As used herein, the term “substrate library” as used here, refers to a set of unique substrate sequences to be used in a peptidase cleavage screening experiment. The library may be designed using various approaches, such as “positional scanning” to test the effect of amino acid substitution at a given position while holding other positions constant within a substrate sequence. A “combinatorial library” approach is one where multiple positions are varied simultaneously within a substrate sequence.

As used herein, the term “sequence diversity” is a quantitative value that is a function of the number of amino acid differences between sequences and the number of unique substrate sequences in a substrate library.

As used herein, an “unbiased library” designed by the Alauna™ method is one that was created with no bias as to what sequences are included. No prior data is required for an unbiased library design, and no known substrate sequences and no previous specificity information is intentionally included in this design. Instead, the unbiased library is designed to include all cooperative interactions at a specified level, containing all possible combinations of amino acids at all possible configurations at two or more positions or sites within a specified length of polypeptide sequence.

As used herein, the term “pharmacological selectivity” pertains to the level of enhanced activity of a drug at a given site, compared to other sites in the body, for example in a diseased tissue as opposed to non-diseased tissues. Disease-selective substrates are those that are selectively cleaved by the peptidase activity from diseased samples versus non-diseased samples. Tissue-selective substrates are those that are selectively cleaved by peptidases in a targeted tissue, versus elsewhere in the body that might also express the molecule targeted by a therapeutic.

As used herein, the term pharmacological “specificity”, pertains to recognition and binding at a molecular level, and is used here to describe the specificity with which a drug binds more tightly to its targeted binding site than to other off-target binding sites.

As used herein, the term “substrate specificity” is used here to describe the recognition of an enzyme or peptidase for its substrate. The substrate specificity for a peptidase pertains to recognition of the amino acids that compose the substrate sequence, and it is a relative value for comparing the cleavability of two or more different substrates that is typically expressed as a ratio of measured values such as relative catalytic rates, catalytic efficiencies, or the total amount of cleavage.

In some embodiments, substrate specificity is a comparative term expressed as a ratio of values, such as rates, efficiencies or total cleavage, that differentiates two or more substrates for their cleavability. In some embodiments, substrate specificity is determined by the “identity” of the amino acids found at each position within the substrate sequence and by the overall conformation of the peptidase substrate.

As used herein, the term “catalytic efficacy” is the concentration at which a defined amount, typically 50%, of the peptidase substrate that is dosed in a chemical reaction or biological assay, is cleaved by a given peptidase preparation under defined reaction conditions. Typically, the goal of catalytic efficacy measurements is to quantify the difference between catalytic efficacy values for two or more unique substrate sequences for cleavage with a single peptidase preparation, measured under defined reaction conditions.

“Catalytic efficiency” is a quantitative term that encompasses both recognition and chemical mechanism features for an enzyme reaction with a substrate. Catalytic efficiency provides a single value for a given peptidase preparation and a given unique substrate sequence, measured under defined “reaction conditions”, which typically include pH, temperature, and buffer composition. A “peptidase preparation” from biological samples may contain one or more individual peptidases that contribute to the total peptidase activity detected in the sample. A “unique” substrate sequence means a unique string of amino acids of a given length that composes the substrate sequence.

As used herein, Gearr™ motifs are defined as the identity of the minimum set of amino acids and their positions within a peptidase substrate sequence that are required to define substrate specificity for an individual sample which contains one or more known or unknown peptidases. Examples of this disclosure are provided for how to build pharmacologically selective peptidase substrates from Gearr™ motifs, and for how to incorporate these into therapeutic and diagnostic molecules.

As used herein, “candidate substrate sequences” are defined as any substrate sequence that is yet untested for cleavage with a peptidase or peptidase preparation. Candidate substrate sequences may be designed to incorporate one or more Gearr™ motifs. A candidate substrate library contains two or more candidate substrate sequences, comprising variations of Gearr™ motifs and their surrounding residues.

As used herein, an amino acid “alphabet” used for library design is the set of amino acids that are components of candidate substrate sequences. A standard alphabet could incorporate the twenty natural amino acids as well as unnatural amino acids. Unnatural amino acids include diverse variants, such as beta amino acids, homo amino acids, derivatives of proline and pyruvic acid, 3-substituted alanine derivatives, glycine derivatives, ring-substituted aromatic amino acids, O-alkylated serines that mimic methionine, N-methyl amino acids, isomers of the natural amino acids such as nor-valine or nor-leucine, d-isomeric amino acids, and others. A reduced alphabet size may also be used, by grouping the amino acids within an alphabet by their physicochemical properties, and randomly selecting an amino acid from within a group for incorporation into the design of a candidate substrate sequence. Such groupings can be defined by any chemical feature of the amino acid, such as low or high molecular weight, acidic or basic character. H-bond donor/acceptor, aromatic or aliphatic, and nucleophilicity or electrophilicity. As used herein, the term “bracketing residues” refers to residues outside of the peptidase substrate sequence. In the context of molecular design for a therapeutic agent or diagnostic probe, these residues may provide structure or other functional support to a peptidase substrate sequence that allows the substrate to be cleaved by peptidases. In the context of a peptidase screening assay, these bracketing residues may be designed to enhance detection and identification of unique cleaved product species within a library by mass spectrometry.

As used herein, “therapeutic index” is defined as the ratio between the concentration at which a drug is toxic and the concentration at which the drug is effective.

As used herein, “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes amino acids which occur naturally by modification of a naturally encoded amino acid (including the 20 common amino acids or pyrrolysine and selenocysteine) but are not themselves incorporated into a growing polypeptide chain by the translation complex. Examples of naturally-occurring amino acids that are not naturally-encoded include N-acetylglucosaminyl-L-serine, N-acetylglucosaminyl-L-threonine, and O-phosphotyrosine. Additionally, the term “non-natural amino acid” includes amino acids which do not occur naturally and may be obtained synthetically or may be obtained by modification of non-natural amino acids.

As used herein, the term “kit” refers to any delivery system for delivering materials. In the context of reaction assays, such delivery systems include systems that allow for the storage, transport, or delivery of reaction reagents (e.g., peptides, oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “solid support” refers to any solid or semi-solid structure suitable for the attachment of biological molecules thereto, such as peptides. Solid supports need not be flat or a single structure, and may be of any type of shape(s) including spherical shapes (e.g., beads). Solid supports may be arranged in any format. In some embodiments, the solid supports are arranged as a microarray (e.g., flat slide), a multiplex bead array, or a well array. In addition, the solid supports may be made of any suitable material, including, but not limited to, silicon, plastic, glass, polymer, ceramic, photoresist, nitrocellulose, and hydrogel. In some embodiments, the solid supports are nitrocellulose, silica, plastic, or hydrogel.

The following non-limiting examples are provided to illustrate various aspects of the present disclosure. All references, patents, patent applications, published patent applications, and the like are incorporated by reference in their entireties herein.

### Low Diversity Screening Efforts for Obtaining Peptidase Cleavable Substrates in the Art

Several peptidase substrate sequences have been identified in the art using low sequence diversity screening efforts. Some exemplary peptidase substrate sequences are listed in Table 1. Although there are numerous peptidase substrate sequences that have been reported in the art, due to certain limitations identified in the current methodologies described below in this disclosure, there is significant need in the pharmaceutical field for identifying peptidase-cleavable substrate sequences that can be engineered into the design of therapeutic and diagnostic molecules for disease- or tissue-selective cleavage.

TABLE 1
Exemplary Peptidases and Peptidase Substrate
Sequences Identified in the Art

| Peptidase | Substrate Sequence† |
|---|---|
| MMP7 | KRALGLPG |
| MMP7 | RPLALWRS |
| MMP9 | PR(S/T)(L/I)(S/T) |
| MMP9 | LEATA |
| MMP11 | GGAANLVRGG |
| MMP14 | SGRIGFLRTA |
| MMP2 | PLGLAG |
| MMP | PLGLAX |
| MMP | ESPAYYTA |
| MMP | RLQLKL |
| MMP | RLQLKAC |
| MMP2, MMP9, MMP14 | EP(Cit)G(Hof)YL |
| uPA | SGRSA |
| uPA | DAFK |
| uPA | GGGRR |
| Lysosomal Enzyme | GFLG |
| Lysosomal Enzyme | (β-Ala)-LAL |
| Lysosomal Enzyme | FK |
| Cathepsin B | L |
| Cathepsin D | PICFF |
| Cathepsin K | GGPRGLPG |
| Prostate Specific Antigen | HSSKLQ |
| Prostate Specific Antigen | HSSKLQL |
| Prostate Specific Antigen | HSSKLQEDA |
| Herpes Simplex Virus | LVLASSSEGY |
| Protease | |
| HIV Protease | GVSQNYPIVG |
| CMV Protease | GVVQASCRLA |
| Thrombin | G-(D-Phe)-(Pip)-RS |
| Thrombin | DPRSFL |
| Thrombin | PPRSFL |
| Caspase-3 | DEVD |
| Caspase-3 | DEVDP |
| Caspase-3 | GDEVDGSGK |
| IL1β converting enzyme | GWEHDG |
| Enterokinase | EDDDDKA |
| FAP | TSGPNQEQK |
| Kallikrein 2 | GKAF-(D-Arg)-R |
| Plasmin | (D-Ala)-FK |
| Plasmin | (D-Val)-LK |
| TOP | (B-Ala)-LAL |
| uPA, matriptase, legumain | LSGRSDNH |
| MMP-9 | VPLSLYS |
| MMP-1 | VLVPMAMMAS |
| Matriptase | XXQAR(A/V)X |
| Matriptase | AGPR |
| Legumain | AANL |
| Legumain | PTNL |
| Matriptase | LSG(R/K) |

†Amino acid sequences are provided in standard one letter code for the natural L-amino acids, and three letter code for the un-natural amino acids D-alanine (D-Ala), D-valine (D-Val), D-phenylalanine (D-Phe), β-alanine (β-Ala), citrulline (Cit), and homophenylalanine (Hof).
Other abbreviations: Piperidine (Pip), methyl (me), and X = any amino acid.

Several substrates in Table 1 were identified from low sequence diversity screening efforts, such as for FAP, legumain and plasmin. The substrate TSGPNQEQK for the peptidase FAP was identified with a candidate-based approach, and is a natural sequence derived from the protein alpha-2-anti-plasmin, a known physiological substrate for FAP (Lo et al., 2009; Edosada et al., 2006). The substrate sequences containing AAN or PTN for legumain were also identified with a candidate-based approach, and were selected from a limited number of alternate substrate sequences in early studies with fluorogenic peptide substrates (Chen et al., 1997; Rotari et al., 2001). The substrate sequences for plasmin, (D-Ala)-FK and (D-Val)-LK, were identified using candidate-based testing for peptidase activity, also within a narrow sequence diversity of fluorogenic substrates (Cs.-szabo et al., 1980; Clavin et al., 1977). PS-SCL was used to identify the substrates DEVD and WEHD for Caspase 3 and IL1β converting enzyme, respectively (Thornberry et al., 1997).

Alternatively, combinatorial display library screening has produced substrates from larger sequence libraries, for example MMP14 and uPA (Table 1). The substrate for MMP14 containing the sequence RIGFLR was identified using a phage display library incorporating six randomized sequence positions, that was screened by ELISA to identify substrate sequences with a maximum yield of cleavage (Kridel et al, 2002). Yet, this highly cleaved substrate shared only two out of six identical residues within the substrate sequence with another substrate sequence containing PLGLQR, that was similarly identified by combinatorial library display in a different system; in the latter case bacterial display was used that explored additional positions in the substrate sequence, and substrates were screened for those that were most rapidly cleaved (Jabaiah and Daugherty, 2011). This demonstrates that the specifications for library design and screening can dramatically affect the output substrate sequence.

Using a similar bacterial display approach, a substrate for uPA (LSGRSDNH) was identified using a library that was described as containing approximately 10⁸ random 8-mer substrate sequences (Stagliano et al. U.S. Pat. No. 9,453,078B2-Modified antibody compositions, methods of making and using thereof. (2016)). Although this library size might seem large, this is less than 1/10,000^{th} of the number of clones needed to fully explore this sequence space (Clackson & Wells, 1994), likely omitting more suitable sequences for rapid cleavage due to limitations in library design. The sequence LSGRSDNH also resulted from serial screening steps, which progressively eliminates sequences from the screening pool at each step (Stagliano et al. U.S. Pat. No. 9,453,078B2—Modified antibody compositions, methods of making and using thereof. (2016)). The surviving sequence LSGRSDNH, was subsequently tested for cleavage by legumain, matriptase or other peptidases in a candidate approach, but these enzymes were not screened against the full library. This demonstrates two key limitations of such combinatorial library approaches for screening a series of individual peptidases. First, if a limited sequence library is used, a potentially large sequence space is omitted, and second, by focusing on identifying only a subset of cleaved substrate sequences in each screening step, essential information for sequence variants in sequence space is lost for identifying the substrates that are more favorably cleaved overall within the set of desired peptidases. Specification of serial screening with limited libraries yields limited results.

These methods, including the candidate-based methods, the partial specificity determination methods such as PS-SCL, and the combinatorial library screening methods, all fall short for addressing the full scope of the selective peptidase substrate identification problem. First, they all assume that targeting individual peptidases with highly specific substrates and selecting substrates with the most complete or most rapid cleavage, will also yield disease- or tissue-selective substrates. Second, current library screening approaches are ultimately limited in the sequence space that they explore and in the information content that they generate, which is a missed opportunity for direct identification of selective peptidase substrates.

In fact, no single peptidase is exclusively expressed and active in a targeted disease tissue, and the concept of finding one perfectly specific substrate for each peptidase is highly unlikely given the broad range of substrates already known for most peptidases (Rawlings et al., 2016). Even if it were possible, a specific substrate that uniquely targets a single peptidase is not guaranteed to yield an optimal disease- or tissue-selective substrate. Instead, the paradigm for disease-selective peptidase substrates is redefined herein as those substrates that are more effectively cleaved in a targeted disease tissue than in non-diseased or healthy tissues. Such substrates will be cleaved by multiple peptidases that act in concert, each with individual rates of cleavage and efficiency, that additively produce an overall cleavage yield that must meet specified levels required for the target application.

On the other hand. The Alauna™ method for peptidase activity profiling is designed to accommodate testing of peptidase preparations from complex biologically-derived samples, and is not limited to screening with individual peptidases. It is through comparative analysis of these complex samples that pharmacologically selective cleavage information can be extracted from multiple samples.

The problem in the art regarding obtaining peptidase substrate sequences is best summarized as simply incomplete and inaccurate information. When substrate sequences are missing from a screening library by design, such as from searching sequence space too narrowly or from biased amino acid composition, or by accident due to individual substrate sequence instability, certain highly cleavable peptidase substrates may be omitted or suppressed by a library screening process. Bias is a known issue in combinatorial display library designs, which employ degenerate codons to produce sequence variants in the DNA that encodes the display proteins. For example, the degenerate NNS or NNK codons used to generate combinatorial display libraries for several of the substrates identified in Table 1, (Matthews and Wells, 1993; Daugherty and Boulware U.S. Pat. No. 7,666,817-Cellular libraries of peptide sequences (CLIPS) and methods of using the same. (2010); Kridel et al . . . 2002; Stagliano et al. U.S. Pat. No. 9,453,078B2-Modified antibody compositions, methods of making and using thereof. (2016)). NNS and NNK degenerate codons encode all twenty of the natural amino acids as well as a single STOP codon, and result in the random incorporation of DNA bases at each NNS- or NNK-encoded position. This method produces incomplete libraries, however, because random insertion of STOP codons can produce artificial truncation products, and random incorporation of cysteines can yield polypeptide species prone to oxidation and destabilization. To compensate, a larger number of transformed clones are routinely prepared to increase the probability that all sequence variants will be encoded in the library (Clackson and Wells, 1994). The common degenerate codon methods also produce biased amino acid compositions due to codon redundancy; specifically, the amino acids R. S and L are incorporated three times more frequently, and the amino acids P, T, V, A, and G are incorporated two times more frequently than the amino acids N, Q. D or E. This requires work-around solutions that can help to reduce bias, such as using computational design of individual DNA primers with alternate degenerate codons (Tang et al., 2012), or designing the libraries with more narrow sequence diversity based on prior knowledge (Mena et al., 2005).

On the other hand, the Alauna™ method for library design uses defined synthetic libraries, rather than random mutagenesis synthetic libraries, to produce sequence libraries that are unbiased in amino acid composition and that completely cover the designed sequence diversity of the library, thereby giving each unique substrate sequence an even chance to compete with others within the library.

Another problem with the PS-SCL and combinatorial display library screens as they are currently practiced, is either incomplete or inaccurate specificity information. For PS-SCL and related methods, only a limited number of positions within a substrate sequence relative to the cleaved scissile bond can be explored, and in the screening process, the sequences of individual substrates are not determined. In combinatorial display libraries, the focus is on sifting through a large library to find the most rapidly or highest yield cleaved substrates. These methods only report out the set of the maximally cleaved clones, not the negative set of clones that were either non-cleaved or that might be defective in the expression system. If certain substrates have non-ideal behavior such as insolubility or they are prone to aggregation, they may be incorrectly assigned to the non-cleaved set, when instead they were absent in the peptidase assay. Low abundance cleavages will also be missed depending on the threshold for detection with these screening approaches. Thus, in most cases, the sequences of individually cleaved substrates are only determined on a limited subset of clones that were enriched or selected by the combinatorial display method, or the sequence information may even be discarded after amino acid frequency information is extracted.

There are currently no published methods available for screening peptidase activity with library-based methods that accurately measure substrate cleavage without bias, limitations in the substrate sequence positions that can be explored, or loss of sequence information in the experimental screening process.

On the other hand, the Alauna™ method provides specifications for library screening to increase the sequence data information content in two ways. First, the Alauna™ method identifies the site of cleavage within a substrate sequence, providing an essential position register required for data analysis. Second, the Alauna™ method specifies identification of sets of unique substrate sequences from across a full range of cleavage efficiencies, from high, intermediate, or low cleavage to the set of non-cleaved substrate sequences. It is the quantitative analysis of the rate and yield of cleavage for these sets of substrate sequences against all the other unique substrates within a library that enables comparison for pharmacologically selective cleavage between targeted disease- and non-diseased or healthy tissues.

Finally, for all of the library screening methods, there are practical technical limits for library size that are currently on the order of 10¹⁰ unique clones in a combinatorial display library using the phage display platform, and 10⁸ using the bacterial display platform. In practice, these combinatorial display methods suffer from codon bias and inefficiencies in library generation, and therefore can cover the full sequence diversity of only five to seven variable sites (FIG. 1) (Clackson & Wells, 1994).

Inefficiencies in library building aside, eight positions are insufficient to define selective peptidase substrates for therapeutic and diagnostic applications, especially if multiple peptidases are to be included in the substrate cleavage. Although many individual peptidases require approximately two amino acid residues to define their substrate specificity, the most specific peptidases often require substrate sequences of six, seven, eight or more residues (Rawlings et al., 2016).

Therefore, for selective peptidase substrate design, it is desirable to have access to larger sequence diversity libraries to accommodate substrate sequences that are eight or more amino acids in length, and that can incorporate multiple substrate sequences for multiple peptidases into hybrid substrate sequences that may be eight to thirty or more amino acids long. Having a more efficient method for the exploration of a larger sequence space with library design is required to achieve these goals.

The Alauna™ method for selective peptidase substrate sequence identification is an entirely novel approach that more efficiently searches sequence space by directly testing cooperativity between sets of amino acids in all positions within substrate sequences. Alauna™ method is analyzed to extract Gearr™ motifs that include both positive and negative specificity information and can be employed in selective peptidase substrate design. This method requires fewer substrate sequences in a well-defined library to screen a larger substrate sequence space, overcoming practical library size limitations, and it provides a more efficient method to discover disease-, tissue-, and/or cell-selective substrate sequences.

### Alauna™ Method

The present invention provides libraries and methods for profiling enzymatic substrate specificity, such as for determining recognition sequences for peptidases. The substrate specificity of a peptidase is an important characteristic that often governs its biological activity. Knowledge of substrate specificity can help to, for example, identify macromolecular substrates for a given peptidase, thus shedding light on its biological activity. Substrate specificity can also guide the design and generation of potent and selective substrates and inhibitors. Therefore, the present invention provides methods and libraries for profiling substrate specificity.

Methods of making the libraries are also provided. As an example, the peptidase substrate libraries of this disclosure are useful in obtaining a complete substrate profile of a peptidase. For example, positional scanning techniques can be employed using the methods and libraries of the invention. The invention provides novel libraries and methods of building them, as well as novel methods of profiling enzymes. For example, a novel profiling method is provided for determining the amino acid preferences for substrate sequences on either side of a cleavage site.

In general, the steps are as follows:

### Step 1

A. Input hypothesis (Input #1) e.g. a therapeutic target for a disease such as a drug target and/or a target peptidase or set of peptidases based on their abundance and activity. (SELECT TARGET)
   1) a drug target such as a molecule or receptor associated with a target disease, tissue, or cell type, and/or
   2) a target peptidase or set of peptidases that are associated with the target disease, tissue or cell type.
B. Set forth Conditions for Input #1: e.g.
   (i) Condition #1: Site of action for the drug.
   (ii) Condition #2: Expression of drug target in non-diseased or in healthy tissues
   (iii) Condition #3: Expression of the peptidase or set of peptidases in healthy tissue.
C. Data Sets: the type of data to be collected for Input #1: e.g.
   (i) Data Set #1. For example, information on target-genetic data, quantitative data etc.
   (ii) Data Set #2. For example, information on substrate;
   (iv) Data Set #3: For example, information on intracellular location, activity etc.
   (v) include genetic data, quantitative data etc.
D. Information from Data Sets is extracted, processed, and scored.
E. Criteria Matrix: Evaluate and revise the input hypothesis (Input #1), and to select, for example, a target peptidase or set of peptidases that are hypothesized to meet a specified level of disease or tissue-selectivity in the context of peptidase-cleavable substrate design.

### Step 2.

A. Conduct discovery experiments on target identified from analysis of Data Sets. Results are identified as Output #'s 1, 2, and 3.
B. Analyze data of each Output #'s 1, 2, and 3.
C. Generate a set of Gearr™ motifs from Output #4 analysis.

### Step 3.

A. Computational design of refined library, using Gearr™ motifs from Output #4 analysis as seed sequences, arranged in all possible permutations.
B. Incorporation of additional sequence variants to generate a more refined library which provides additional specificity information about the amino acid preferences at additional positions within a Gearr™ motif.

### Step 4.

A. Refined library production.
B. Conduct screening experiments on samples containing target identified from analysis of Data Sets. Results are identified as Output #S.
C. Analyze data of Output #S for selectivity.
D. Generate a set of cleavable substrate sequences. Results are identified as Output #6.
   Steps 3 and 4 can be repeated to generate further refinement.

Accordingly, in some embodiments, Alauna™ method is useful for the discovery and development of peptidase cleavable substrate sequences.

In some embodiments, Alauna™ method is useful for the discovery and development of disease-, tissue- and/or cell-selective peptidase cleavable substrate sequences.

In some embodiments, Alauna™ method comprises one or more component methods that have multiple routes to obtain a substrate sequence, depending upon the selectivity required for these substrates. An exemplary linear pathway through these methods is drawn in FIG. 3. Broadly, this method progresses from hypothesis generation (Step 1), to discovery (Step 2), to design (Step 3) and evaluation (Step 4).

In some embodiments, Alauna™ method comprises experimental methods for the collection of and computational methods for the use of existing data to support each step in the process, and/or a therapeutic agent or a diagnostic tool development program.

In one aspect, a method of building peptidase cleavable substrate library is provided, the method comprises the steps of:
(vi) Providing an unbiased library of peptides or proteins, wherein the peptide or protein comprises a substrate sequence, and the design of the library is the result of mathematical modeling to assess all possible cooperative interactions between at least two, three or more amino acid residues within a given sequence space for a defined number of residues and length of substrate sequence;
(vii) Incubating the library in the presence of the peptidase, allowing the peptidase to cleave peptides or proteins within the library to form a population of cleaved and non-cleaved peptides or proteins;
(viii) Screening the population of cleaved and non-cleaved peptides or proteins for obtaining Gearr™ motifs;
(ix) Extracting Gearr™ motifs using a computational algorithm; and
(x) Building a refined peptidase cleavable substrate sequence library from Gearr™ motifs, wherein the substrate sequences are capable of being cleaved by at least one peptidase.

In another aspect, provided herein is a method of obtaining a peptidase cleavable substrate comprising the steps of:
(iii) Building a peptidase cleavable substrate library as described above in this disclosure;
(iv) Selecting individual peptidase cleavable substrate comprising at least one Gearr™ motif, wherein the substrate is capable of being selectively cleaved by at least one, at least two, or at least three peptidases.

In another aspect, provided herein is a method of obtaining disease-, tissue- and/or cell-selective peptidase cleavable substrate, comprising the steps of:
(v) Obtaining a peptidase cleavable substrate as described above in this disclosure;
(vi) Optionally, identifying bracketing residues, wherein bracketing residues are located at upstream and/or downstream positions outside of a candidate disease-, tissue- and/or cell-selective peptidase cleavable substrate;
(vii) Contacting the candidate peptidase cleavable substrate with at least one disease-, tissue- and/or cell-selective peptidase;
(viii) Evaluating and validating cleavage of the candidate substrate sequence, whereby obtaining the disease-, tissue- and/or cell-selective peptidase cleavable substrate.

In some embodiments, obtaining disease-, tissue- and/or cell-selective peptidase cleavable substrate sequence is useful for therapeutic and/or diagnostic applications.

In some embodiments, obtaining disease-, tissue- and/or cell-selective peptidase cleavable substrate sequence is for use in therapeutic agents and/or diagnostic tools.

Hereafter, the method of obtaining peptidase cleavable substrate sequence or the method of obtaining disease-, tissue- and/or cell-selective peptidase cleavable substrate sequence is referred as Alauna™ method.

The Alauna™ method is a novel approach that efficiently searches sequence space by directly testing cooperativity between sets of amino acids in all positions within candidate peptidase cleavable substrate sequences.

Alauna™ method requires fewer substrate sequences in a well-defined library to screen a larger substrate sequence space, overcoming practical library size limitations, and it provides a more efficient method to discover disease-, tissue- and/or cell-selective peptidase cleavable substrate sequences.

In some embodiments, the screening of the population of cleaved and non-cleaved substrates comprises quantitively measuring the abundance of cleavage products at different time points by sequencing methods.

In another embodiment, the screening of the population of cleaved and non-cleaved substrates further comprises analysis and identification of the scissile bond for each individual peptide by sequencing methods.

In some embodiments, the sequencing methods are selected from mass spectrometry-based proteomic analysis, differential fluorescence, differential immunodetection, next generation sequencing techniques, or combination thereof.

In some embodiments, Alauna™ method comprises a method for substrate sequence library design to maximize sequence diversity within the library that directly tests cooperative interactions, allowing for more efficient exploration of the full sequence space available for peptidase substrate design.

In some embodiments, the Alauna™ method comprises a screening step to achieve accurate substrate specificity information.

In some embodiments, substrate specificity information is analyzed to identify Gearr™ motifs, which are defined herein as the identity of the minimum set of amino acids and their positions within a peptidase substrate sequence that are required to define substrate specificity at a specified threshold for an individual sample which contains one or more known or unknown peptidases. Examples are provided for how to build pharmacologically disease-, tissue- and/or cell-selective peptidase cleavable substrates from Gearr™ motifs, and for how to incorporate these into therapeutic agents and diagnostic tools.

In some embodiments, the Alauna™ method comprises one or more component methods that have multiple routes to obtain a final peptidase substrate, depending upon the selectivity required for these substrates. In some embodiments, positional scanning techniques can be employed using the methods and libraries of the invention.

According to one aspect of the invention, the Alauna™ method comprising library screening step increases the sequence data information content.

In one embodiment, screening step comprises analyzing the population of cleaved and non-cleaved peptides or proteins.

In one embodiment, the Alauna™ method specifies that the site of cleavage within a candidate substrate sequence is identified, providing an essential position register required for data analysis.

In another embodiment, the Alauna™ method comprises identification of sets of unique candidate substrate sequences from across a full range of cleavage efficiencies, from high, intermediate, or low cleavage to the set of non-cleaved substrate sequences. It is the quantitative analysis of the rate and yield of cleavage for these sets of candidate substrate sequences against all the other unique candidate substrates within a library that enables comparison for pharmacologically selective cleavage between targeted disease- and non-diseased or healthy tissues.

In some embodiments, peptidase cleavable substrate library is a peptide or protein library containing a series of peptides or proteins of different sizes ranging from 2 amino acids to 200 amino acids. In some embodiments, the peptidase cleavable substrate library is a mixed library containing peptides or proteins of different sizes.

### Unbiased Libraries and Screening Methods

In some embodiments, The Alauna™ method for library design comprises use of defined synthetic libraries, rather than random mutagenesis synthetic libraries, to produce sequence libraries that are unbiased in amino acid composition. Alauna™ method is useful for covering the designed sequence diversity of the library, thereby giving each unique substrate sequence an even chance to compete with others within the library.

The goal of unbiased library design is to arrange all of the amino acids within the defined sequence space to maximize the number of unique Gearr™ motifs that are represented in the library. As the length of the peptidase-cleavable substrate is allowed to increase, this increases the number of ways that unique Gearr™ motifs can be configured within the sequence, and the number of different contexts in which the unique Gearr™ motifs are presented within the library.

In some embodiments, unbiased library size is determined by considering the following parameters to define the sequence space of the library: (i) the number of positions allowed to vary within the linker sequence, (ii) the number of positions to be included in a Gearr™ motif, and (iii) the composition of amino acids that may be used in the substrate. These amino acids are then systematically arranged within the substrate sequence space to maximize the number of unique Gearr™ motifs that can be generated at various positions across the linker to generate an unbiased library.

In some embodiments, the unbiased library is for identifying the Gearr™ motifs.

In some embodiments, a library design approach incorporates cooperative interactions between two, three or more residues. The unbiased library is one that includes all possible combinations of amino acids arranged in all possible configurations within a given sequence space for a defined number of residues and length of substrate sequence.

In some embodiments, screening methods comprises the analysis of at least two, three, four or more subsets of substrate sequences with high, moderate, and low cleavage as well as the subset of sequences that were not detected to be cleaved.

In some embodiments, the screening methods may utilize a detection platform such as differential fluorescence, differential immunodetection, or next generation sequencing (NGS) or mass spectrometric sequencing methods that can quantitatively measure the abundance of cleavage products at time points during the reaction. The kinetic analysis of these data may use standard enzymatic activity modeling to calculate either the catalytic efficiency (k_{cat}/K_{M}), the catalytic rate (typically measured as Vₘₐₓ or as an initial rate or an observed rate k_{obs}), or the catalytic efficacy (measured as the percentage yield of substrate consumed or product formed) at a fixed time point in the reaction.

In some embodiments, the subsets of the library may also be subjected to substrate sequencing at the DNA or protein level in order to determine the full-length substrate sequences, using NGS sequencing if a biological display platform is used to build the substrate library, or using mass-spectrometry based proteomic analysis to identify the substrate sequences at the protein level if the substrate library is synthetic.

In some embodiments, the site of cleavage. i.e. the scissile bond, is identified for each individual substrate sequence using mass spectrometry-based proteomic analysis for each of the cleaved subsets of the library, in order to provide an essential register for the sequence analysis of cleaved products.

In some embodiments, the unbiased library is a peptide or protein library containing a series of peptides or proteins of different sizes ranging from 2 amino acids to 200 amino acids. In some embodiments, the unbiased library is a mixed library containing peptides or proteins of different sizes.

In some embodiments, the unbiased library can be composed of any natural or unnatural amino acids.

In one embodiment, the unbiased library of polypeptide sequences was designed using the Alauna™ method to be composed of the following nineteen amino acids: A, D, E, F, G, H, I, K, L, M, N, P, Q, R, S, T, V, W, and Y, excluding C due to its propensity to oxidize. The contribution of each amino acid to the overall composition of this library was 1/19, or 5.26±0.15%. The number of positions that were designed to vary was ten, and each amino acid appeared at each position within the substrate sequence library with an average positional frequency of 5.26±0.15%, as shown in FIG. 6, panel A. Furthermore, the library was designed to include all three-position Gearr™ motifs within a maximum length of nine positions, and resulted in the Alauna™ method unbiased library 3.9, with a library size of 2×10⁵ unique substrate sequences within the unbiased library design.

In some embodiments, peptide sequences in unbiased library are synthesized using standard solid phase peptide synthesis methods. Any method for the production of polypeptide sequences that bear these sequences may also be used to generate the library, including other chemical methods such as solution phase peptide synthesis and native chemical ligation methods, cell free translation and protein synthesis, recombinant technologies for polypeptide expression, or genetically encoded protein or peptide library display.

In some embodiments, substrate sequences in unbiased library are built within the context of a biological display system that can be chosen from phage, bacterial or mammalian expression systems. In this scenario, the substrate sequences for an Alauna™ method library is incorporated into the sequence of a surface-expressed scaffold protein. The library is designed on the protein level with amino acid sequences, which specify the synthesis of oligonucleotide sequences that encode them. Synthetic oligonucleotide primers are designed using the principles of molecular cloning, such as using Gibson cloning technology (Gibson et al., 2009) to assemble overlapping gene fragments and effectively insert the synthetic oligonucleotides encoding an Alauna™ method library into a biological display platform system. Once the surface display system is built, the completeness of the library is tested using NGS genomic sequencing, by focusing the sequencing experiment on the region containing the Alauna™ method library sequence insert. The goal for quality control of the system is that the library is 99.9% complete, which is an important feature of the Alauna™ method data analysis.

In some embodiments, unbiased library design is applied to the analysis of amino- or carboxy-peptidases. Examples of these important peptidases include cathepsin B, FAP, dipeptidyl peptidase IV (DPPIV and other peptidases that process important signaling cascades, such as those that control peptide hormone processing, immunopeptide processing for presentation by MHC, or that control disease mechanisms such as the alpha, beta and gamma secretases which process amyloid precursor protein into the A-beta form that forms plaques in Alzheimer's disease. These peptidases may be direct targets of therapeutic intervention or they may be a means for activation or inactivation of a therapeutic molecule. In all cases, if they are intended to cleave a polypeptide bond that is adjacent to a small molecule, unbiased libraries can be designed with these peptidase substrate sequences in mind. These molecules have a very large accessible sequence space, because they may also be synthesized using solid phase peptide chemistry incorporating unnatural amino acids. The design parameters for such a substrate sequence include: the number of variable positions within the synthetic substrate sequence, the number of variable positions outside of the synthetic substrate sequence, the position of the covalent attachment between the synthetic substrate sequence and the therapeutic moiety, and the position of the covalent bond to the small molecule.

In some embodiments, Alauna™ method for substrate screening comprises identification of the scissile bond from the data analysis.

In some embodiments, the preferred method for site identification is using peptide-based sequencing by mass spectrometry. Modern instrumentation uses tandem liquid chromatography-mass spectrometric (LC-MS/MS) detection, typically employing reversed phase chromatography for separation and positive ion mode detection in most experiments. In this case, it is important to design peptidase substrates for the screening assay that are also amenable to detection by LC-MS/MS.

### Gearr™ Motifs

The term “Gearr™ motif” is defined here as the minimal set of two or more amino acid positions within an enzyme substrate sequence that interacts with the enzyme(s), in which the identity of the amino acids present has a measurable and significant positive or negative effect upon the rate of scissile bond cleavage by the enzyme(s). In certain embodiments, the enzyme is a peptidase. By considering all possible combinations of amino acids in this way, it is possible to test for cooperative interactions.

In some embodiments, extracting Gearr™ motifs using a computational algorithm comprises measuring cooperativity between two or more unique amino acids found at specific positions within the substrate sequences of a library.

In some embodiments. Gearr™ motifs comprise both positive and negative specificity information.

In some embodiments, Gearr™ motifs are useful for obtaining peptidase cleavable substrate. In other embodiments, Gearr™ motifs are useful for obtaining disease-, tissue- and/or cell-selective peptidase cleavable substrate.

The amino acid positions within a Gearr™ motif are numbered with index numbers that increase with distance from the scissile bond, as diagrammed in FIG. 4. In the N-terminal direction, the Gearr™ motif positions are numbered M₁, M₂, M₃, M₄ and so on, and in the C-terminal direction, the motif positions are numbered M₁′, M₂′, M₃′, M₄′ etc.; thus, the scissile bond for a peptidase is between motif positions M₁ and M₁′. For example, the Gearr™ motif for human trypsin is defined by positions M₁ and M₁′, in which 99% of its known substrate sequences contain either Arg or Lys in M₁, and less than 1% of its substrates cleave with a Pro in position M₁′ (Rawlings et al., 2016). Although trypsin is an endopeptidase and has a substrate binding pocket that accommodates longer substrate sequences, the positions outside of M₁ and M₁′ are not part of this Gearr™ motif because substitutions with any of the twenty natural amino acids at these sites produce substrate sequences that all can be cleaved within a range of catalytic efficiency values that are within approximately 10-fold of each other (Clavin et al., 1977). Thus, the result produced by identifying a Gearr™ motif is to provide a simple set of rules for substrate sequence design for a peptidase.

In some embodiments, the concept of redundancy is used to describe the number of times each Gearr™ motif is represented within the unbiased library. For example, the pair of residues (G, P) was represented 60 times in the Alauna™ method unbiased library 3.9 when A=Gly and B=Pro and they were found in adjacent positions, denoted as AB. The same pair of Gly and Pro residues found in positions AXXB (where X=any amino acid, and positions A and B were separated by two X residues) had a redundancy of 47. The redundancy, or number of ways that individual amino acid pairs can be represented within the substrate library diminishes as the distance between residues A and B increases, given the limited configurations with which these pairs can be arranged in a given space, as shown in FIG. 6. On average, all 361 AB pairs of amino acids for this library were represented with an average redundancy of 59±8, and for AXXXXXXB pairs it was 20±6 (FIG. 6, panel B).

If a peptidase had a strict requirement for Gly-Pro in positions M₂ and M₁, it would be presented with 60 suitable peptide sequences within this library. One skilled in the art will recognize that those peptides that cleave more efficiently will likely be enriched for GP in positions M₂ and M₁, and also that the Gly-Pro peptides in AB pairs that are less efficiently cleaved will have disfavored residues in other positions outside of M₂ and M₁. In the same library, the Gly-Pro pair was designed to be represented in the context of each of the other 19 amino acids at each adjacent position such as M₁′ or M₂′ on average 2-3 times. Thus, alignment of the cleaved peptide sequences by their scissile bond position would reveal the favored and disfavored residues represented in the peptidase specificity motif at other positions such as M₁′ or M₂′ in a peptidase cleavage experiment.

In some embodiments, a substrate sequence is required that is maximally selective for one targeted peptidase over other peptidases that may be present in a desired sample type to be assayed, such as in a diagnostic or reporter assay. With prior knowledge about the targeted peptidase, a Gearr™ motif size might be hypothesized to be composed of a certain number of positions that are required to describe its specificity.

The structure of the diagnostic or reporter assay might also impose some constraints on the available sequence space, as may be the case with fluorogenic substrates that employ fluorescence resonance energy transfer (FRET) or internal quenching groups, or with activatable cell imaging probes (ACPP) that contain extended polypeptide sequences outside of the substrate sequence. These substrates can dictate the design of the substrate sequence by requiring that some individual positions within the sequence are fixed by the probe design, while others are allowed to be varied, for example as diagrammed in FIG. 2. In such a case, the scissile bond of the Gearr™ motif is designed within the activatable region of the substrate probe. The unbiased substrate library can be fit around the fixed features of these probes, and the resulting Gearr™ motifs obtained may span the labeled structural features.

For in vivo applications, “pharmacological selectivity” pertains to the selective cleavage of a peptidase substrate within a targeted disease-tissue, at a specified level above background cleavage in healthy or non-diseased tissue. This requires a broader range of substrate sequences that can be cleaved by multiple peptidases within the targeted disease-tissue. Such substrate sequences may be hybrid substrates with Gearr™ motifs that share one or more positions, or they may incorporate multiple Gearr™ motifs that are placed in tandem to fit within a given number of amino acid positions, such as up to twenty or thirty positions. The limit for the substrate sequence length is often affected by other features of the molecular design, such as a required interdomain distance within the larger polypeptide design for a therapeutic molecule. What is important to understand, is that as the length of a peptidase substrate increases, the total sequence space that describes it also increases; a simple peptidase substrate sequence containing eighteen amino acids would require a sequence space of 20¹⁸=2.6×10²³ total unique sequences to describe it, an astronomical value greater than the number of stars in the observable universe. The Alauna™ method provides a method for unbiased library design that allows for efficient exploration of Gearr™ motifs spanning such distant sites. For example, cooperative interactions between 4 residues spanning a distance of up to 18 positions can be tested with the Alauna™ method unbiased library 4.18, consisting of 3.0×10⁷ sequences (FIG. 1). The ability to test distant sites enables applications to tandem Gearr™ motif design, for example by testing the effects of bracketing residues or upstream or downstream Gearr™ motifs on the efficiency of cleavage for a given candidate substrate sequence.

In some embodiments, as the substrate sequence length increases, this also increases the number of arrangements for Gearr™ motifs within that length. One approach is to directly test a longer substrate sequence with all possible Gearr™ motifs designed within it for screening. An unbiased library composed of all possible four-position Gearr™ motifs with a maximum distance of nine positions could be arranged into a longer substrate sequence containing eighteen variable positions, with an unbiased library size of 2×10⁶ unique substrate sequences. To achieve the selectivity required for this problem, targeted diseased samples and non-diseased or healthy samples would be screened with an unbiased library (FIG. 3. Step 2), resulting in a matrix of values for all possible Gearr™ motifs tested against all screening samples (FIG. 3, Experiment 2). Those Gearr™ motifs that meet specifications for selectivity under desired conditions can be built into a hybrid or tandem arrangement of Gearr™ motifs within a substrate sequence of any length as needed for a particular design (FIG. 3, Step 3).

In some embodiments, Gearr™ motifs may span the fixed position of the substrate, thus an unbiased library may be used to explore cooperative interactions between the nearby residues and the synthetic substrate sequence. If a substrate sequence for such a design was designated by two variable positions, and twenty natural amino acids and 20 un-natural amino acids could be used in this design, the number of unique substrate sequences just within the substrate itself include 40²=1,600 possible combinations. If a Gearr™ motif is composed of three positions that can span the fixed site of the covalent attachment between synthetic substrate and the therapeutic moiety, and only the three nearest positions are considered to be able to interact, the approximate library size would be 1.2×10⁵ unique substrate sequences to efficiently explore a sequence space given by 1.28×10⁷ possible unique sequences. Thus, consideration of Gearr™ motifs in the design of such a library provides a 100× more efficient method for searching sequence space.

For other molecular designs that incorporate a peptidase substrate sequence, such as lipids, poly-saccharides, or oligonucleotides, each of these derivatives is attached to a polypeptide molecule at a fixed position. The Gearr™ motifs surround this position for pro-drug design, and the sequence space is increased by the ability to synthesize the covalently attached synthetic substrate sequences with unnatural amino acids.

Extracting Gearr™ Motifs and Building Selective Peptidase Cleavable Substrate Sequence Library from Gearr™ Motifs

In some embodiments, extraction of Gearr™ motifs is performed by using a computational algorithm that can measure the cooperativity between two or more unique amino acids found at specific positions within the substrate sequence. One method for the calculation of cooperativity is to determine the frequency of co-occurrence of two or more unique amino acids at specific positions within the substrate sequence as compared to the distribution of co-occurrences within the overall library.

In some embodiments, Gearr™ motifs is useful to design novel cleavable substrate sequences that can be selectively cleaved by two or more peptidase preparations.

Peptidase preparations can range from single purified recombinant enzymes to complex mixtures of peptidases that are extracted from patient-derived tissues and biofluids, or that are produced from cell culture or other biological models from lysates, extracts or other forms of conditioned media that contain peptidases.

In some embodiments, a method for the selection of patient-derived or model system-derived healthy and diseased samples is provided. The method can be used for the positive and negative screening conditions required to build disease-tissue-, and/or cell-selective peptidase cleavable substrate sequences. A criteria matrix is used for weighing source data that can approximate the abundance of both the drug target and of the active peptidases to be used for pro-drug or reporter substrate cleavage across diseased and healthy tissues.

In some embodiments, building peptidase cleavable substrate sequence library from Gearr™ motifs further comprises testing a candidate peptidase cleavable substrate within a sample, wherein the sample is selected based on a criteria matrix.

### Criteria Matrix for Sample Selection

In some embodiments, building peptidase cleavable substrate library from Gearr™ motifs comprises testing a candidate peptidase cleavable substrate within a sample, wherein the sample is selected based on a criteria matrix.

In other embodiments, contacting the candidate peptidase cleavable substrate with at least one disease-, tissue- and/or cell-selective peptidase comprises testing a candidate peptidase cleavable substrate within a sample, wherein the sample is selected based on a criteria matrix.

In some embodiments, the criteria matrix guides sample selection.

In some embodiments, the criteria matrix is based on the molecular target for a therapeutic drug design, and the healthy and diseased tissues where the target is expressed.

In some embodiments, the criteria matrix comprises scored measurements for i) co-expression of peptidases with the drug target at the therapeutic site of action versus in other sites, ii) peptidase specificity, iii) maximum peptidase catalytic activity, and iv) specific co-localization of peptidases with the drug target at the therapeutic site of action.

The therapeutic site of action is specified by the disease status and the location of the molecular target in the body with features such as organ, tissue, compartment, cell type, and subcellular localization. The specification for the therapeutic site of action affects sample selection for measurement of protein abundance and peptidolytic activity at that site.

In some embodiments, sample is selected from a diseased tissue, non-diseased or healthy tissues.

In some embodiments, diseased tissue comprises a therapeutic target.

### Bracketing Residue Library Design

Once Gearr™ motifs have been identified using screening of an unbiased library, or in a scenario when a known substrate sequence is intended to be validated, a small library of unique sequence variants may be designed for such sequences using a candidate-based approach.

Provided herein, is a method for identification of bracketing residues immediately upstream and downstream of the cleavable substrate sequence insertion site into a therapeutic, diagnostic or other reporter molecule.

In some embodiments, identification of bracketing residues comprises consideration of longer-range cooperative interactions with residues from component Gearr™ motifs. Bracketing residues are also used to optimize a scaffolding platform such as a polypeptide for the presentation of candidate substrate sequences.

Provided herein is also a method for the evaluation and validation of cleavage of candidate substrate sequences that include one or more Gearr™ motifs. These new cleavable substrate sequences can be evaluated within the context of a larger biomolecule such as a protein therapeutic or scaffold protein or other molecule, or within a reporter probe bearing a candidate substrate sequence, using mass-spectrometry based proteomic methods, differential fluorescence, or immuno-selection coupled with separation or spectroscopic detection.

In some embodiments, an algorithm was used for substrate design that assists in the design of bracketing residues outside of the peptidase substrate sequence, since the candidate sequences themselves cannot be altered just for detection purposes of the assay. The bracketing residues outside of a candidate substrate sequence serve multiple purposes. In a therapeutic construct they serve as the non-structured connection between a substrate sequence and the rest of the polypeptide molecule, but in mass spectrometry, these amino acids can be used to adjust the net charge, polarity or hydrophobicity of the peptide substrates to make them amenable to analysis, such as by improving solubility or ionization. Furthermore, these residues contribute mass to the molecule, and can serve as unique mass tags that can be resolved by the instrument, which enables identification of the peptide fragments that are produced by peptidase digestion.

In some embodiments, Alauna™ algorithm for bracketing residue library design performs multiple theoretical substitutions of the bracketing residues. Rules for substitution include selecting from a set of amino acids that are suitable for use as a flexible linkage and which do not cause significant changes to the structure of the peptide. In the same way as an unbiased library is designed to maximize sequence diversity, so too is the sequence diversity maximized for a bracketing residue library. However, additional calculations require that the resulting combinations also produce suitable mass tags and enhance solubility and ionization of the intact peptide as well as its cleavage products.

Provided herein, Alauna™ method for identification of “disease selective” substrate sequences by measuring and comparing the catalytic efficiency of substrate cleavage for multiple candidate substrate sequences, using peptidase preparations from diseased and non-diseased samples; those substrates with greater catalytic efficiency in the diseased condition than in the non-diseased or healthy condition are considered “disease-selective”. The result of this process is the identification of unique substrate sequences that contain one or more Gearr™ motifs corresponding to each of the individual peptidases involved, which together produce cleavages at one or more scissile bonds, each with a different catalytic efficiency value. Thus, catalytic efficiency is useful for comparison of a single substrate sequence or peptidase substrate, tested across multiple peptidase preparations and reaction conditions.

Provided herein, Alauna™ method for identification of “disease specific” substrate sequences by measuring and comparing the catalytic efficacy of substrate cleavage for multiple peptidase substrates with a single diseased sample. A preferred result from the Alauna™ method can be the identification of “non-cleavable” and “cleavable” unique substrate sequences that differ in catalytic efficacy by a defined amount of substrate cleavage. A “non-cleavable” substrate sequence is one that produces an undetectable or otherwise insignificant amount of cleaved product, at a value that is preferably less than 5% or 1% of the total peptidase substrate, as measured with a particular detection method such as immuno-detection or mass spectrometry under defined assay conditions within a specified length of time. A “cleavable” substrate sequence is one that is capable of being cleaved at a specified level that is matched to the required yield of cleaved product release for a given application, typically greater than or equal to 90%, or greater than 99%, for a specified amount or dose of peptidase substrate, as measured in the same assay. To maximize the specific cleavage of a peptidase substrate, the goal is to produce at least a 100-, 1000-, or 10,000-fold difference between the catalytic efficacy of cleavage for a cleavable substrate versus a non-cleavable substrate. The greater the fold-difference for catalytic efficacy between cleavable and non-cleavable substrates, the greater the induction effect that may be achieved with the peptidase activation mechanism for a drug or reporter probe.

The Alauna™ method applies these concepts to identify substrate sequences that are disease selective using catalytic efficiency, and the level of induction that can be achieved using peptidase activation is measured using catalytic efficacy.

The Alauna™ method provides specifications for library screening to increase the sequence data information content. In some embodiments, the Alauna™ method identifies the site of cleavage within a substrate sequence, providing an essential position register required for data analysis. In some embodiments, the Alauna™ method specifies identification of sets of unique substrate sequences from across a full range of cleavage efficiencies, from high, intermediate, or low cleavage to the set of non-cleaved substrate sequences. It is the quantitative analysis of the rate and yield of cleavage for these sets of substrate sequences against all the other unique substrates within a library that enables comparison for pharmacologically selective cleavage between targeted disease- and non-diseased or healthy tissues.

In some embodiments, the Alauna™ method for selective peptidase substrate sequence identification searches sequence space by directly testing cooperativity between sets of amino acids in all positions within substrate sequences. The output data from the Alauna™ method for substrate screening is analyzed to extract Gearr™ motifs that include both positive and negative specificity information and can be employed in selective peptidase substrate design. This method requires fewer substrate sequences in a well-defined library to screen a larger substrate sequence space, overcoming practical library size limitations, and it provides a more efficient method to discover disease- or tissue-selective substrate sequences.

### Peptidases

In some embodiments, the peptidases are selected from the group of endopeptidases, omega-peptidases, exopeptidases, aminopeptidases, carboxypeptidases, dipeptidyl-peptidases, tripeptidyl peptidases, peptidyl dipeptidases, dipeptidases, or combination thereof.

In some embodiments, the peptidases are extracted from human subject-derived tissues and/or biofluids, cell cultures, transgenic cellular expression systems, or animal models for human disease and biological systems.

In some embodiments, the peptidases are prepared from lysates, extracts, biofluids or conditioned media, with or without further purification.

### Methods of Use for Therapeutic and Diagnostic Applications

The prodrugs have fewer side effects, better in vivo pharmacokinetic profiles (e.g., longer half-life) and better target specificity, and are more efficacious as compared to prior therapeutics.

In one aspect, the present disclosure provides prodrug conjugates that are metabolized in vivo to become active therapeutics. In some embodiments, prodrug conjugates comprise a peptidase-cleavable substrate obtained by Alauna™ method.

In one aspect, provided herein is a prodrug conjugates capable of releasing a drug upon activation by one or more peptidases.

In some embodiments, the prodrug conjugate comprises a therapeutic moiety, a masking moiety, a cleavable peptide or protein linker, optionally a carrier moiety, optionally a targeting moiety and optionally a half-life extension moiety, wherein the cleavable peptide or protein linker is peptidase cleavable substrate or disease-, tissue- and/or cell-selective peptidase cleavable substrate obtained by Alauna™ method.

In some embodiments, the masking moiety binds to the therapeutic moiety and inhibits a biological activity of the therapeutic moiety, the therapeutic moiety is optionally fused to the carrier moiety, and the masking moiety is fused to the therapeutic moiety or optionally to the carrier moiety through a cleavable peptide or protein linker.

In some embodiments, the prodrug conjugate comprises a therapeutic moiety, a carrier moiety, wherein the carrier moiety comprises a cleavable peptide or protein linker, the cleavable peptide or protein linker is peptidase cleavable substrate or disease-, tissue- and/or cell-selective peptidase cleavable substrate obtained by Alauna™ method, and the therapeutic moiety is encapsulated into the carrier moiety.

In some embodiments, the therapeutic moiety is effective as an anti-inflammatory agent, an antiallergic, an antibiotic, an anticancer agent, an antidiabetic, an antiviral agent, an antihypertensive agent, an antianginal, an anticonvulsant, an analgesic, an antiasthmatic, an antidepressant, an antidiarrheal, an anti-infective agent, an antimigraine agent, an antipsychotic agent, an antipyretic agent, an antiulcerative agent, an antithrombotic or combination thereof.

The term “therapeutic moiety” will be used in its broadest sense to include any moiety capable of providing a desired or beneficial effect on living tissue. Therapeutic moieties include, but are not limited to pharmaceutical drugs, vaccines, antibodies, proteins, peptides and nucleic acid sequences (such as supercoiled, relaxed, and linear plasmid DNA, antisense constructs, artificial chromosomes, or any other nucleic acid-based therapeutic), and any formulations thereof.

In some embodiments, the carrier moiety may be selected from lipid-based carrier systems such as anionic (conventional) liposomes, pH sensitive liposomes, immunoliposomes, fusogenic liposomes, neutral lipid nanoparticles, charged lipid nanoparticles, and charged lipid/antisense aggregates.

In some embodiments, the targeting moieties of the prodrug conjugates may be an antigen-binding moiety, or a moiety that is not an antigen-binding moiety. The targeting moieties may target to a specific healthy or disease site in the body, such as a tumor site.

In some embodiments, the half-life extension moiety may improve the pharmacokinetic profiles such as serum half-life of the prodrug conjugate.

In one aspect, the prodrug is a cytokine prodrug. In some embodiments, the cytokine prodrug comprises a cytokine moiety, a masking moiety, and optionally a carrier moiety, wherein the masking moiety binds to the cytokine moiety and inhibits a biological activity of the cytokine moiety, the cytokine moiety is fused to the carrier moiety, and the masking moiety is fused to the cytokine moiety or to the carrier moiety through a peptidase-cleavable substrate.

In some embodiments, the therapeutic moiety is a cytokine moiety and the masking moiety is a cytokine antagonist.

The cytokine antagonist, which may be, for example, an extracellular domain of a receptor for the cytokine, is linked to the cytokine moiety or to the carrier moiety through a cleavable linker (e.g., peptidase-cleavable substrate identified by Alauna™ method). The mask inhibits the cytokine moiety's biological functions while the mask is binding to it. The prodrugs may be activated at a target site (e.g., at a tumor site or the surrounding environment) in the patient by cleavage of the linker and the consequent release of the cytokine mask from the prodrug, exposing the previously masked cytokine moiety and allowing the cytokine moiety to bind to its receptor on a target cell and exert its biological functions on the target cell. In some embodiments, the carriers for the prodrugs are antigen-binding moieties, such as antibodies, that bind an antigen at the target site.

In some embodiments, the cytokine moiety is a human IL-6, IL-10, TNF-alpha, or interferon (IFN)-gamma antagonist polypeptide.

In one aspect, pro-drugs provided herein, comprises (i) a “masking moiety” that regulates the active form of the drug, and (ii) a “cleavable moiety” that can release the mask. Peptidase activity can be used to produce cleavages in the cleavable moiety for the release of pro-drug forms of small molecule drugs, macromolecular biologics, as well as synthetic nanoparticles and other non-polypeptide therapeutic or diagnostic molecules.

In some embodiments, the cleavable moiety is a peptidase-cleavable substrate. In a preferred embodiment, the peptidases that are active in targeted tissues provide enhanced selectivity for the pro-drug by producing cleavage and release of the active drug that is enhanced at the target site as compared to other sites in the body.

In one aspect, the prodrug is a small molecule pro-drug. For small molecule pro-drugs, a peptidase-cleavable substrate may also serve as a mask that interferes with the drug's ability to bind to its target, or to access the biological compartment or tissue where its target is localized. In some embodiments, the small molecule may be tethered by the peptidase-cleavable substrate to a larger macromolecule such as a protein domain that serves to enhance other pharmacological features of the drug, such as its solubility or stability.

In one aspect, the pro-drug is a macromolecular pro-drug. In some embodiments, a peptidase-cleavable substrate may be used for the activation or inactivation, delivery, stabilization, and/or unmasking of a macromolecular drug. Use of a peptidase-cleavable linker can provide an additional layer of specificity to the drug design, by selecting a peptidase-cleavable substrate sequence that is preferentially cleaved at the target site, and that is not cleaved in non-diseased tissues.

In one aspect, the masking moiety of a pro-drug can be applied to a drug to reduce its toxic effects by preventing binding to the drug target, or by preventing the drug from entering an undesired tissue or subcellular compartment, or by preventing the drug from adopting a conformation that allows it to be active. When the mask is removed, the active form of the drug is revealed and able to perform its function. This reduces the toxicity of the drug overall, and improves its therapeutic index. The converse scenario, where a drug design includes a peptidase-cleavable inactivating motif, can also improve therapeutic index. In this latter design, a cleavable substrate can serve as an “off” switch to prevent an active drug from causing toxicity in an undesired tissue or subcellular compartment, such as may be due to prolonged activity or to undesired accumulation in a non-targeted tissue or subcellular compartment.

In one aspect, peptidases are used to enhance the selectivity of pro-drug forms of chemotherapy agents including monoclonal antibodies, ADCs, bi-specific and tri-specific T-cell engagers.

In one aspect, peptidases are used to enhance the selectivity of pro-drug forms of monoclonal antibodies, by releasing a mask that prevents the antibodies from binding to the recognition target of the antibody, that prevents the antibody from being recognized by an Fe receptor, other antibodies or antibody fragments, or that prevents the antibody from adopting a conformation that allows it to be active. When the mask is removed, the active form of the antibody is revealed and able to perform its function. This reduces the toxicity of the antibody, and improves its therapeutic index.

In one aspect, peptidases are used to enhance the selectivity of pro-drug forms of Bi-specific T-cell engagers (BiTEs), by selectively releasing a mask that otherwise prevents binding of the BiTE to one or both of its recognition targets, or that prevents the BiTE from adopting an active conformation. When the mask is removed, the active form of the BiTE is revealed and able to perform its function. This reduces the toxicity of the BITE, and improves its therapeutic index.

In one aspect, peptidases are used to enhance the selectivity and stability of pro-drugs comprised of single-chain variable fragments (scFv). Peptidase cleavage is used to release a masking group, that itself may be an scFv, and when released, this reveals the active molecule. Peptidase cleavage may also be used to release a stabilizing domain such as albumin or an albumin binding domain, such that upon cleavage and release from the stabilizing domain, the drug has a faster clearance rate, reducing its toxicity and overall improving the therapeutic index.

In one aspect, peptidases are used to enhance the selectivity and stability of pro-drugs comprised of chimeric antigen receptor T-cell therapy (CAR T), which incorporates a single-chain variable fragment (scFv) as the extracellular antigen recognition domain of the CAR. Patients receiving CAR T therapy are subject to a high frequency of adverse events called cytokine release syndrome (CRS), and immune effector cell associated neurotoxicity syndrome (ICANS). Adoptive T cell therapy can also have unpredictable expansion of the CAR T cells once given to the patient, making dosing difficult to control and leading to toxicity. Using a peptidase cleavage sequence to release a masking group in the presence of targeted tissues or diseased tissues would reduce these toxic events by keeping the CAR T cells in check.

In some embodiments, the present disclosure provides inducible polypeptides. In some embodiments, the inducible polypeptide is a conditionally inducible polypeptide. In some embodiments, inducible polypeptides comprise a peptidase-cleavable substrate obtained by Alauna™ method.

In some embodiments, peptidase-cleavable substrate is incorporated into the inducible polypeptide, wherein peptidase-cleavable substrate is capable of being cleaved by at least one peptidase.

In some embodiments, peptidase-cleavable substrate is incorporated into the inducible polypeptide, wherein peptidase-cleavable substrate is capable of being cleaved by at least one disease, tissue- and/or cell-selective peptidase.

In some embodiments, T-cell engagers e.g. Bi-specific T-cell engagers (BiTEs) comprises peptidase-cleavable substrate or disease, tissue- and/or cell-selective peptidase cleavable substrate obtained by Alauna™ method.

In some embodiments, T-cell engagers e.g. Bi-specific T-cell engagers (BiTEs) are fused to a peptidase-cleavable substrate or disease, tissue- and/or cell-selective peptidase cleavable substrate obtained by Alauna™ method.

In some embodiments, peptidases are used for the selective cleavage of polypeptide sequences that are designed into reporter substrates, that when cleaved either activate, inactivate, or otherwise release a fragment that generates spectroscopic signal, e.g. from a UV-Visible dye, fluorophore, or Raman active dye. Peptidase cleavage would release two or more tethered molecules comprised of any combination of small molecules, polypeptides, oligonucleotides, lipids, synthetic polymers, or assemblies of these molecules used in therapeutic or diagnostic applications.

In one aspect, the present disclosure provides a method for obtaining disease, tissue- and/or cell-selective peptidase cleavable substrates for use in nanosensors.

In some embodiments, nanosensors are used to detect cancer.

In one aspect, provided herein is a nanosensor comprising a carrier moiety, a detectable marker, and a disease, tissue- and/or cell-selective peptidase cleavable substrate obtained by Alauna™ method, wherein the peptidase cleavable substrate is linked to both the carrier moiety and the detectable marker, and the peptidase cleavable substrate is susceptible to cleavage by a disease-tissue- and/or cell associated protease.

In some embodiments, peptidase cleavable substrate is susceptible to cleavage by a protease associated with cancer, tissue injury or damage, cardiovascular disease, arthritis, viral, bacterial, parasitic or fungal infection, Alzheimer's disease emphysema, thrombosis, hemophilia, stroke, organ dysfunction, any inflammatory condition, vascular disease, parenchymal disease, or a pharmacologically-induced state.

The carrier domain may serve as the core of the nanoparticle. A purpose of the carrier domain is to serve as a platform for the peptidase cleavable substrate. As such, the carrier can be any material or size as long as it can serve as a carrier or platform. Preferably the material is non-immunogenic, i.e. does not provoke an immune response in the body of the subject to which it will be administered. Another purpose is that it may function as a targeting means to target the modular structure to a tissue, cell or molecule. In some embodiments the carrier domain is a particle. A particle, for example, a nanoparticle, may, for instance, result in passive targeting to tumors by circulation. Other types of carriers, include, for instance, compounds that cause active targeting to tissue, cells or molecules. Examples of carriers include, but are not limited to, microparticles, nanoparticles, aptamers, peptides (RGD, iRGD, LyP-1, CREKA, etc.), proteins, nucleic acids, polysaccharides, polymers, antibodies or antibody fragments (e.g., herceptin, cetuximab, panitumumab, etc.) and small molecules (e.g., erlotinib, gefitinib, sorafenib, etc.)

As used herein the term “particle” includes nanoparticles as well as microparticles. Nanoparticles are defined as particles of less than 1.0 μm in diameter. A preparation of nanoparticles includes particles having an average particle size of less than 1.0 μm in diameter. Microparticles are particles of greater than 1.0 μm in diameter but less than 1 mm. A preparation of microparticles includes particles having an average particle size of greater than 1.0 μm in diameter. The microparticles may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiment, the diameter is about 10 nm to about 100 nm.

The particles may be composed of a variety of materials including iron, ceramic, metallic, natural polymer materials (including lipids, sugars, chitosan, hyaluronic acid, etc.), synthetic polymer materials (including poly-lactide-coglycolide, poly-glycerol sebacate, etc.), and non-polymer materials, or combinations thereof.

The carrier may be composed of inorganic materials. Inorganic materials include, for instance, magnetic materials, conductive materials, and semiconductor materials.

In addition to particles the carrier may be composed of any organic carrier, including biological and living carriers such as cells, viruses, bacteria, as well as any non-living organic carriers, or any composition enabling exposure of enzyme substrates to enzymes in disease (including extracellular, membrane-bound, and intracellular enzymes).

The detectable marker is capable of being released from the nanosensor when exposed to a peptidase in vivo or in vitro. In some embodiments, the detectable marker once released is free to travel to a remote site for detection. A remote site is used herein to refer to a site in the body that is distinct from the bodily tissue housing the peptidase where the cleavage occurs. In some embodiments, the bodily tissue housing the peptidase where the cleavage occurs is a tumor. In some embodiments, a remote site is a biological sample that is non-invasively obtained from a subject, for example a urine sample, or a blood sample.

Cleavage of the peptidase cleavable substrate by a peptidase in vivo, results in the production of the detectable marker. Alternatively, cleavage of the peptidase cleavable substrate by a peptidase in vitro, results in the production of the detectable marker.

In some embodiments, the detectable marker is composed of two ligands joined by a linker. The detectable marker may be comprised of, for instance one or more of a peptide, nucleic acid, small molecule, fluorophore/quencher, Raman active dye, carbohydrate, particle, radiolabel, MRI-active compound, inorganic material, organic material, with encoded characteristics to facilitate optimal detection (e.g. compound, ligand encoded reporter, ligand encoded reporter, or isotope coded reporter molecule (iCORE)).

In some embodiments, the detectable marker comprises a fluorescence resonance energy transfer (FRET) pair, wherein the FRET pair comprises a fluorophore molecule and a quenching molecule, optionally wherein the fluorophore molecule and the quenching molecule flank the enzyme susceptible domain.

### Kits

In certain embodiments, the unbiased libraries are provided in a kit. The peptides can be attached to a solid support, such as, a microarray, tissue culture plates, beads and the like or can be supplied separately as in a fragmented kit.

In certain embodiments, the peptides are attached to a solid substrate.

In certain embodiments, the peptides are provided in solution or dried form.

In certain embodiments, the kit is configured for high-throughput screening (HTS) assays. In HTS methods, many peptides can be tested in parallel by robotic, automatic or semi-automatic methods so that large numbers of peptides are screened for a desired activity simultaneously or nearly simultaneously. It is possible to assay and screen up to about 6,000 to 20,000, and even up to about 100,000 to 1,000,000 different compounds a day using the integrated systems of the invention.

In certain embodiments, it may be desirable to immobilize either the peptides, enzymes or to accommodate automation of the assay. Binding of, for example, a peptide library can be accomplished in any vessel suitable for containing the reactants. Examples of such vessels include microtiter plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or receptor protein, and the mixture incubated under conditions conducive to complex formation (e.g., at physiological conditions for salt and pH). Following incubation, the beads or microtiter plate wells are washed to remove any unbound components.

In the high throughput assays, either soluble or solid state, it is possible to screen up to several thousand different peptides in a single day. For a solid state reaction, the peptide library can be bound to the solid state component, directly or indirectly, via covalent or non-covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder. A number of tags and tag binders can be used, based upon known molecular interactions well described in the literature. Similarly, any haptenic or antigenic compound can be used in combination with an appropriate antibody to form a tag/tag binder pair. Thousands of specific antibodies are commercially available and many additional antibodies are described in the literature

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39 (4): 718-719 (1993); and Kozal et al., Nature Medicine 2 (7): 753759 (1996) (all describing arrays of biopolymers fixed to solid substrates). Non-chemical approaches for fixing tag binders to substrates include other common methods, such as heat, cross-linking by UV radiation, and the like.

### EXAMPLES

### Example 1: Disease-Selective Cleavable Substrate Sequence Identification and Method of Use

The Alauna™ method was applied to the selection of therapeutic targets for the immune oncology indication.

Input hypothesis (FIG. 1. Input #1) Drug target selection: In one embodiment, the drug target comprises a target antigen selected from but not limited to any of the following: CD19, CD20, CD33, CD30, CD64, CD123, EpCAM, EGFR, HER-2. HER-3, c-Met, LAG3, FoIR, PSMA, VEGF, and CEA. In one aspect, a target antigen is an immune checkpoint protein. Immune checkpoint proteins include but are not limited to CD27, CD40, OX40, GITR, CD137, B7, CD28, ICOS, A2AR, H3, B7-H4, BTLA, CTLA-4, IDO, KIR, LAG3, PD-2, PD-L1, TIM-3, and VISTA. Examples of inhibitory immune checkpoint proteins to be inhibited in activating an immune response, include but are not limited to, A2AR, B7-H3, B7-H4, BTLA, CTLA-4, IDO, KIR, LAG3, PD-1, PD-L1, TIM-3, and VISTA. In some embodiments, binding of the therapeutic agent to an immune checkpoint target protein is dependent upon peptidase cleavage of a substrate sequence that releases a masking domain, which restricts binding of the therapeutic agent to the immune checkpoint target protein only at the therapeutic site of action, as in a tumor microenvironment.

Peptidase target selection: The human genome encodes over 550 peptidases (Puente, 2003). Any of these peptidases can serve as target peptidases for pro-drug design, including several with previously developed peptidase cleavable substrate sequences (Table 1). Among these peptidases, the matrix metalloproteases (MMPs) have well-described roles in carcinogenesis, and can serve as useful prognostic markers for cancer (Egeblad and Werb, 2002; Turunen et al., 2017). The input hypothesis regarding peptidase selection for this embodiment was that one or more MMPs, alone or in combination with other peptidases, would serve as useful peptidase targets for multiple solid tumor indications. To narrow the selection of peptidases and drug target, data sets #1-4 as described in the Alauna™ method (FIG. 1, Input #1) were assembled to build a criteria matrix as follows:

Data set #1, drug target and peptidase abundance. A recent analysis of the gene expression profiles from the TCGA data set for cancer markers found that the MMPs in general are highly upregulated in a majority of 15 cancer cell types surveyed (Gobin et al., 2019). This gives confidence to consideration of MMPs, but for hypothesis refinement, the level of correlation with the drug target was assessed at the gene expression level within the TCGA data set (Gao et al., 2019, 2013), accessed at (https://portal.gdc.cancer.gov/). During hypothesis refinement, analysis of data from the TCGA data set showed that MMP14 ranked in the top 25% of overexpressed genes in 11 out of 18 lung cancer datasets and 8 out of 16 colorectal cancer datasets, and it was not apparently differentially regulated in breast cancer. Differential regulation of a peptidase at the abundance level between tumor and non-diseased tissues can add to an improvement in the therapeutic index of a peptidase-activated pro-drug.

Proteomic (histopathological) and transcriptomic analyses of tumor and normal tissue in the Human Protein Atlas (version 18.1, release date 2018 Nov. 15) (Fagerberg et al., 2014; Uhlén et al., 2015) reveal additional information on broad expression profiles of the MMPs and other candidate peptidases across tissue types. Among multiple MMPs considered during hypothesis refinement, the Human Protein Atlas data showed that MMP14 is overexpressed in tumor tissue compared to normal tissue specifically in the lungs, breast, skin and pancreas. MMP14 is broadly expressed across multiple tissue types at low levels.

A large microarray dataset collected from over 1000 human cancer cell lines, accessible from the Genomics of Drug Sensitivity in Cancer (GDSC) project (Iorio et al., 2016) was queried for the correlation mRNA expression levels for peptidases with the candidate drug targets listed above. The drug target vascular endothelial growth factor (VEGF) showed a positive correlation of expression with MMP14 in multiple kidney, bone, thyroid and skin cancer cell lines. Additional peptidases that correlated with VEGF were cathepsin B, ADAM9, legumain, and uPA, providing an opportunity to fine-tune specificity of peptidase cleavage in individual tissues. VEGF functions in paracrine signaling; therefore, expression patterns of the VEGF receptor 1 and 2 were also investigated. VEGF receptor 1 additionally correlated with MMP14 expression in skin.

VEGF is a secreted protein that is stored by association with extracellular matrix, and it is released from the matrix through the activity of extracellular peptidases; different levels of MMP activity affect the pattern of angiogenesis promoted by VEGF in the tumor microenvironment (Lee et al., 2005). Additionally, there is evidence that MMPs can directly cleave VEGF that affects its adherence and release from the extracellular matrix space (Lee et al., 2005). MMP14 has also been reported to directly cleave VEGF receptor 1 in cornea (Han et al., 2016). Direct cleavage of the VEGF receptor 1 or 2 and of matrix-bound VEGF offers two alternate mechanisms for MMP involvement in regulating VEGF signaling.

Data set #2, peptidase substrate information. At the outset of the project, one criterion for selection of a target peptidase or family of peptidases is the probability that a new peptidase cleavable substrate sequence can be identified that will meet requirements for disease-selective activation. The Alauna™ method does not strictly require a rational hypothesis that selects a peptidase target at the start of a project. Thus, consideration of Data set #2 is optional, but information on the specificity of a targeted peptidase can be added to the substrate refinement process (FIG. 3, Step 3), and when a peptidase target is likely to be highly specific, this adds confidence that the process will yield the specified disease-selective activation required for a therapeutic application.

In this embodiment, Data set #2 included substrate sequence information from the MEROPS database for MMP14 as well as other MMPs (Rawlings, 2016; Rawlings et al., 2004). A consensus sequence of PXG|L, where the “|” indicates the scissile bond, was found for MMP14 in the MEROPS database, although the source studies for this data were not readily available in the database. Biological display methods have been reported elsewhere for MMP14 (Jabaiah and Daugherty, 2011; Kridel et al., 2002), as well as other candidate substrate library approaches (Turk et al., 2001), which, although each approach suffers from some limitations or sequence bias resulting in different consensus sequences, each showed amino acid preferences in at least three sites within the substrate sequence, indicating a probability that specific and selective substrates could be obtained for MMP14 among other MMPs. Other MMPs also cleave a similar PXG|L consensus sequence, but other sites outside of these positions have been shown to contribute to specific cleavage with MMP14 vs other peptidases. e.g. (Turk et al., 2001) that suggest that further MMP specificity can be obtained.

Data set #3 included enzyme activity data aggregated within the BRENDA database (accessed November 2019) for MMP14. The BRENDA database lists several natural substrates, such as collagen type I alpha chain and pro-MMP2, consistent with its role in promoting degradation of the extracellular matrix and in activating other MMPs.

Typical kinetic parameters, measured with tool substrates, were listed with low nanomolar Michaelis constants (K_{M}), and piocomolar K_{M} values were measured for collagen I alpha-1 and -2 chain substrates. The fluorescent probe methoxycoumarin-4-acetyl-Lys-Pro-Leu-Gly-Leu-Lys (2,4-dinitrophenyl)-Ala-Arg-NH₂ had a reported turnover number of 0.33-6.8 (s⁻¹), and catalytic efficiency values of 550-2429 mM⁻¹ s⁻¹ measured by fluorescence assay. All of these values are comparable to MMP9 values, measured toward similar substrates as reported in the BRENDA database.

Data set #4, subcellular localization: MMP14, as a member of the membrane-type matrix metalloproteases (MT-MMP1), is localized to the cell surface. This membrane localization is attractive for application to tumor-targeting pro-drug design as this would predict more restricted localization than secreted forms of MMPs and other peptidases. MMP14 also has a sheddase function that releases proteins from the cell surface, similar to the A Disintegrin and metalloproteinase domain-containing (ADAM) family of peptidases; known sheddase substrates of MMP14 include EMMPRIN, kidney injury molecule I, MHC class I chain-related molecule A, and other regulated proteins (Egawa et al., 2006; Guo et al., 2012; Liu et al., 2010).

### Selection of Conditions #/1, #2, and #3.

MMP14 is a marker of poor prognosis in pancreatic cancer (Määttä et al., 2000), colorectal cancer (Cui et al., 2019), breast cancer (Radisky and Radisky, 2015), and ovarian cancer (Kamat et al., 2006). MMP14 is also a candidate positive prognostic biomarker for diffuse large B-cell lymphoma, and higher concentrations of MMP14 are correlated with increased immune cell infiltration, in particular M0 macrophages (Yin et al., 2020). VEGF is a prognostic marker in renal cancer (unfavorable), liver cancer (unfavorable), endometrial cancer (unfavorable), urothelial cancer (favorable) and cervical cancer (unfavorable). It is an intracellular and a secreted protein, and has low tissue specificity. Therefore, adding a layer of specificity to a therapeutic agent that targets VEGF is a strategy to improve therapeutic index for these agents. The tissues and cell lines at the intersection of MMP14 and VEGF expression and colocalization define Condition #1. Based on the indications listed above, the list of possible disease tissue types where MMP14 and VEGF may both be targeted for pro-drug design of anti-VEGF therapeutics includes: pancreatic, colorectal, breast, ovarian, renal, liver, endometrial, urothelial, and cervical cancers. To demonstrate drug efficacy, it is preferred to utilize cell lines that are suitable for animal xenograft experiments downstream of the Alauna™ method.

The cell lines utilized for the GDSC microarray analysis were used to narrow selection to kidney, colon, thyroid and skin cancer cell lines that may be suitable positive cell lines in terms of abundance for both MMP14 and VEGF or VEGF receptors. In one embodiment, these cell lines represent Condition #1 in the screening assay design for Step 2 of the Alauna™ method. Matched isogenic negative control cell lines are also used for cell-based experiments, using genetic knock-outs built with CRISPR or knock-down for expression levels using siRNA technology, to serve as Condition #2 or Condition #3 samples that are missing either VEGF or VEGF receptor, or MMP14, respectively. From the GDSC analysis, the thyroid carcinoma cell lines ASH-3, FTC-133, KS and BCPAP; and the renal carcinoma cell lines RCC-MF, KMRC-1, A498 were both positive for both MMP14 and VEGF and represent samples from Condition #1. The malignant melanoma cell lines IST-MEL1 and CP50-MEL-B are also predicted to be strongly positive for MMP14 and VEGF receptor-1 (fms-related tyrosine kinase 1). In the generic example, selection of cell types that are amenable to xenograft are recommended to generate a physiological experimental system.

Anti-VEGF and anti-VEGFR monoclonal antibody therapy is in use for metastatic colorectal cancer, metastatic breast cancer. NSCLC, or gastric cancer, renal cancer, hepatocellular cancer, thyroid cancer, as well as macular degeneration. Adverse effects related to the action of these agents for oncology applications include thrombosis, bleeding, proteinuria, endocrine dysfunction, and hypertension (Ferrara et al., 2005; Kamba and McDonald, 2007). Adverse events with anti-VEGF therapy for ocular treatments also include gastrointestinal disorders and thromboembolytic events, particularly in older patients (Falavarjani and Nguyen, 2013). Anti-VEGF therapy is not recommended for women of reproductive age, due to systemic side effects in the mother and high risk of fetal harm (Rogers et al., 2016). These adverse events can be connected to normal physiological functions of VEGF signaling, and inform the selection of sample types for Condition #2: tissue samples from normal placenta, thyroid, gastrointestinal/colon, kidney, breast, lung, liver, and ocular tissues or model cell systems.

Broad screening across patients is introduced at Step 4 of the Alauna™ method. The screening should be performed with selected tissues and cell lines from among samples of colorectal, kidney, pancreatic, breast, ovarian, DLBCA, bone, thyroid, placenta, skin, liver, and other cell types of cancers and matched normal tissues or cell lines.

To supplement Data sets #1 and #4, histology data for tissue co-localization of MMP14 and VEGF and VEGF receptor proteins is used to prioritize the candidate tissues with the highest likelihood of co-localization. Tissues from this list that are negative for extracellular MMP14 represent Condition #2; tissues that are positive for MMP14 but negative for VEGF or VEGF receptors represent Condition #3. In addition, to assess stability of a candidate MMP14 cleavable substrate sequence, counter screening for Condition #3 can include samples prepared from healthy compartments such as circulatory peptidases in serum or plasma (Jambunathan and Galande, 2014).

Discovery Experiments, Alauna™ method Step 2 (FIG. 3).

In this embodiment, the target peptidase or set of peptidases was selected from the family of MMP enzymes, specifically MMP14.

Experiment 1 is a multiplexed substrate profiling assay performed using purified recombinant enzymes. A suitable Alauna™ method unbiased library was selected by matching to the required level of complexity for the application. In this embodiment, the MMP family of peptidases, and specifically MMP14 have been selected as the peptidase target(s). The MMP family of peptidases share consensus sequences that can be summarized as PX(Sm)(Hy), where “Sm” are small residues such as G, A, S, N, E and “Hy” are hydrophobic residues such as L, M, Y. I. F (Turk et al., 2001).

It is an ongoing challenge to identify highly “specific” substrates, meaning substrates that are cleaved more rapidly, more efficiently, or more completely, than other substrates by an individual MMP family member. It is similarly difficult to identify, highly “selective” substrates, meaning substrates that is more rapidly, more efficiently, or more completely cleaved by one MMP versus another member of the MMP family using existing technologies. This is because all existing technologies essentially collapse the information collected from a set of profiled peptidase substrates into consensus motifs that summarize amino acid frequency at each position in the sequence, ignoring the cooperativity, i.e. the connectivity, between amino acids within those motifs. Nevertheless, the data collected to date for the MMP family indicates that the individual MMPs require at least three positions to define a substrate that can be recognized and cleaved (Turk et al., 2001).

To determine the specificity of individual peptidases from the MMP family, the Alauna™ method unbiased library 2.4 was used in a multiplexed substrate profiling assay performed in Experiment 1. For this experiment, recombinant enzymes (R&D Systems, #911-MP and #918-MP) were activated according to manufacturer's recommendations. The enzymes were assayed at pH 7.4 in 50 mM TRIS HCl buffer containing 1 mM CaCl₂, at low nanomolar concentrations, typically 10 nM, to catalyze cleavage of synthetic peptides within the Alauna™ method unbiased library 2.4. The synthetic peptides that compose the Alauna™ method unbiased library 2.4 were prepared by standard solid-phase peptide synthesis, and they were combined into a single pool of substrates, prepared at 500 nM equimolar concentration for each substrate in the peptidase reaction. The peptidase assays were performed in a kinetic format, removing aliquots and quenching the reaction with 1% formic acid at multiple time points over the course of the reaction, ranging from minutes to hours. These aliquots were immediately desalted using C18 desalting tips and standard mass spectrometry compatible solvents (Solvent A: 0.1% formic acid in HPLC grade water, Solvent B: 50% acetonitrile. 0.1% formic acid in water), and then dried under a vacuum prior to analysis by LC-MS/MS. Thus, for data analysis it is possible to apply enzyme kinetics principles to assess the observed rate of cleavage (k_{obs}), catalytic efficiency (k_{cat}/K_{M}), and maximum yield of substrate cleavage (% Substrate consumption or % Product formation). In this case, a plateau in maximum product formation (% P) was used as a threshold for binary determination of whether or not a unique bond cleavage occurred after a fixed reaction time. The raw data output of this analysis, (Output #1. FIG. 3) is a list of cleaved sequences aligned by their scissile bond.

In this embodiment, the Alauna™ method unbiased library 2.4 was sufficient to identify Gearr™ motifs composed of more than three amino acid positions for the MMPs by solving for pairs of cooperative interactions that were enriched among the cleaved substrates compared to the total library distribution of possible pairwise interactions. Shown in FIG. 7 are the sequence logo representations of the results of this experiment for MMP14 and for MMP9. In the Experiment 1 for MMP14, there were 80 observed unique substrate cleavages within the Alauna™ method unbiased library 2.4, as measured with mass spectrometry. Among these cleavages were four pairs of cooperative residues that were enriched as compared to the background library sequences, indicating favorable cleavages. All of these cooperative interactions can be summarized with the Gearr™ motif PXG(M/L/I) Y for MMP14. In the experiment with MMP9, 241 unique cleavages were observed, with six pairs of cooperative interacting residues that were enriched at least 2-fold over background, and that resulted in a Gearr™ motif of PG(S/G)(I/R/M/L)XS for MMP9 (FIG. 7).

Therefore, the result of Experiment 1 is a candidate Gearr™ motif that summarizes all of the cooperative interactions observed at a certain threshold, in this case >2-fold enrichment over background, that were measured for a peptidase activity experiment under specified conditions. These Experiment 1 results clearly demonstrated that a Gearr™ motif is distinct from a standard consensus sequence or consensus motif obtained with standard methodology in the peptidase field. A Gearr™ motif captures the connectivity between residues as a co-association that is described as cooperativity, whereas a consensus sequence aims to summarize the amino acid frequency observed at all positions within a substrate sequence, ignoring their connectivity.

Experiment 2 is a biological discovery experiment performed using patient-derived as well as cell-based samples, representing Conditions #1, #2 and #3 as defined by the Input hypothesis (FIG. 3, Input #1).

The first step of Experiment 2 is the preparation of conditioned media samples with flash-frozen surgical biopsy samples of approximately 100 mg tissue mass. Tissue samples from a first series of discovery screens for Condition #1 included pathologically-graded colorectal cancer, and papillary thyroid cancer, as well as matched non-diseased tissue samples obtained from the same procedures (Condition #2 and #3). To test other types of normal healthy tissues, primary tissues and immortalized “healthy” cell lines are used, such as may be collected from colon or thyroid nodule biopsy (graded benign), as well as human renal and colon epithelial cells.

For Experiment 2, conditioned media are prepared from tissue that has been thawed and rinsed twice with phosphate buffered saline (PBS) to remove any visible blood from the sample, prior to dicing them into approximately 1 mm³ pieces. These are rinsed in a warm basal cell culture medium such as Dulbecco's Modified Eagle's medium (DMEM) without supplemented FBS, which can contain factors that alter the peptidase profiles of the samples, and then the media is replaced with a typical volume of 1:30, tissue wet weight to medium volume. The tissue is incubated in the medium under standard mammalian cell culture conditions for 16 hours, then the conditioned medium is collected and replaced with fresh medium for another 16 hours, and combined with the first to produce neat conditioned medium. The tissue may be saved for processing into a cell lysate as well, in case additional markers are to be tested with immunodetection or proteomic analysis. Cytotoxicity is routinely tested in these samples by the lactose dehydrogenase assay, aiming to maintain the samples in culture for up to 3 days, as long as cell death remains <5%. The conditioned medium is then buffer exchanged and concentrated with cell culture-grade PBS. The final total protein concentration is determined in the conditioned medium by BCA assay, and typically is in the range of 1-5 mg/ml.

These conditioned medium samples represent the input for Experiment 2. For proteomic analysis, approximately 10 micrograms of total protein from the buffer-exchanged, conditioned medium was digested with sequencing grade trypsin and subjected to semi-quantitative, label-free proteomic analysis using liquid-chromatography tandem mass spectrometry (LC-MS/MS). Proteomic data were output with spectral counts as an approximation of relative protein abundance. Peptide counts were normalized by the total counts from a single MS sample. Samples are analyzed individually, and the resulting protein identifications were output with spectral counts as an approximation of relative protein abundance. To correct for sample loading differences, the spectral count measurements were normalized to total signal in the sample, missing values were imputed, and the normalized spectral count data were log₂-normalized for ratiometric comparison between samples. Individual comparisons can be made between samples, as well as between sets from Condition #1 versus Condition #2 or #3 using a Welch's t-test in the R statistical computing environment. The peptidases identified in the individual samples inform selection of additional peptidases for Experiment #1. These data also are used to supplement Data set #1 (FIG. 3. Input #1). Proteomic analysis of a set of papillary thyroid tumor surgical samples revealed multiple cathepsins including C′TSB, and the membrane associated peptidase PRSS1 to be up-regulated in the tumor sample compared to a non-diseased adjacent tissue.

Accordingly, CTSB and PRSS1 were selected for Experiment 1 substrate profiling with the Alauna™ method unbiased library 2.4, using the same approach as was used for the MMPs. Human CTSB and PRSS1 (R&D Systems, 953-CY and 3848-SE) were activated according to the manufacturer recommendations. PRSS1 was tested using the Alauna™ method with assay conditions matching the pH 7.4 reactions for MMP14 and MMP9 (50 mM TRIS-HCl, 1 mM CaCl₂, pH 7.4). CTSB activity is increased under acidic reaction conditions such as pH 4.5-5.5, and was therefore assayed at pH 6.0 in 50 mM sodium acetate to enhance its activity. Neither CTSB nor PRSS1 show any significant cooperative interactions in their cleavages obtained by the Alauna™ method, therefore no true Gearr™ motifs can be obtained for these enzymes. However, there is an enrichment in amino acid frequencies that can be represented by the simple consensus sequences: (K/R)|X (where X is not Pro) for PRSS1, and (K/F)(K/R)|(Hy)(G/A) for CTSB. Thus, the specificity of these two enzymes is compatible for a hybrid cleavage motif design.

The potentially active CTSB and PRSS1 peptidases present in the papillary thyroid tumor microenvironment may contribute to the overall peptidase cleavage activity in the sample, contributing cleavages C-terminal to basic residues K and R.

Peptidase activity profiling in Experiment 2 was performed using buffer-exchanged conditioned media from the same set of surgically resected papillary thyroid tumor samples to investigate differential activities observed in tumor versus non-diseased adjacent tissue, using the Alauna™ method unbiased library 2.4. Since the enzyme titer in each sample is unknown, the reactions were normalized by using a fixed total amount by mass of protein from the conditioned media samples as the peptidase sample, and the reactions were performed in a kinetic format to assess enzyme-catalyzed cleavage. As in Experiment 1, a plateau in maximum product formation (% P) was used as a threshold for binary determination of whether or not a unique bond cleavage occurred after a fixed reaction time in each sample. The resulting cleavages from each reaction in Output #3 produced 154 endo-peptidolytic cleavages within this library that were selectively cleaved in the tumor conditioned media sample as compared to the non-diseased adjacent tissue. The raw data output of this analysis, (Output #3. FIG. 3) is a list of cleaved sequences aligned by their scissile bond from each conditioned media sample, among which cooperative interactions were identified using Algorithm 1. The tumor-selective Gearr™ motif YPTL|(I/F)YX(H/N) was identified for these papillary thyroid tumor samples (FIG. 8), and it incorporates six cooperative interactions that were enriched in this set of cleavages over background in the Alauna™ method unbiased library 2.4.

In this embodiment, since VEGF is a secreted protein, the stability of an anti-VEGF pro-drug in circulation is an important pharmacokinetic feature, thus susceptibility of the substrate sequence to cleavage by non-targeted peptidases in healthy tissues or compartments, such as hepsin from liver or the circulating enzymes factor Xa and thrombin, can be considered to be some of the active peptidases from Condition #3. Hepsin is a transmembrane serine peptidase with a strong preference for cleavage C-terminal to Arg residues. Thrombin has a Gearr™ motif of (G/P)(R/K)| and Factor Xa has a slightly broader Gearr™ motif of (P/G/A/L)(R/K)|S. None of these enzymes, when assayed in Experiment 1 with the Alauna™ method unbiased library 2.4, share cooperative pairs of residues with the tumor selective Gearr™ motif for papillary thyroid cancer samples.

In this embodiment, the main application of Algorithm 1 is to perform differential analysis of sets of cleaved substrate sequences obtained from Experiments 1 and 2, Output #1 and #3. The conditions defined by the Input #1 help to define which sets of cleavages are to be compared. In order to ascertain whether the non-diseased tissues qualify as negative for presence of the drug target (representing Condition #2) or for presence of the target peptidase (Condition #3), the relative abundance of these proteins is determined using immuno-detection or proteomic analysis at the protein level. The activity for MMPs in general can also be quantified and compared between samples using highly sensitive enzymatic assays such as by using the fluorescent substrate methoxycoumarin-4-acetyl-Lys-Pro-Leu-Gly-Leu-Lys (2,4-dinitrophenyl)-Ala-Arg-NH₂ to detect MMP activity and define Condition #3.

Output #4 from Algorithm 1 is the tumor-selective Gearr™ motif YPTL|(I/F)YX(H/N). This Gearr™ motif comprises multiple derivative sequences that have a range of kinetic rates and overall yields of cleavage. Since the conditioned media samples contain multiple peptidases, a single variation within a substrate sequence can affect the kinetics of all peptidases that recognize the Gearr™ motif within it. Therefore, lead candidate substrate sequences should be empirically tested to verify and quantify cleavage. In this embodiment, catalytic efficiency is measured because it allows comparison of selectivity for cleavage between individual samples and conditions. In Step 3, all possible variants are calculated for this Gearr™ motif, which includes 76 possible substrate sequences comprised of the lead substrate sequence wherein position M₁′ is an I or F, position M₃′ is one of 19 natural amino acids (excluding C due to oxidation), and position M₄′ is an H or N. In order to reduce the complexity of these 76 variants, a small library is prepared for Experiment 3 prior to starting the Step 3 process of new library design with Algorithm 2. In Experiment 3, the catalytic efficiency of cleavage for the 76 variants of the tumor selective Gearr™ motif are tested to prioritize a smaller number of lead candidate substrate sequences.

Based on proteomic analysis, additional peptidases beyond MMPs were also identified that were differentially up-regulated in tumor versus matched non-diseased samples for papillary thyroid cancer, including PRSS1 and CTSB. Using the consensus motifs for these enzymes identified from Experiment 1 with the Alauna™ method unbiased library 2.4, it was decided at Input #2 to test whether addition of these substrate cleavage sites could enhance the rate or efficiency of cleavage for the candidate substrates. Therefore, configurations were designed using Algorithm 2 that also incorporate candidate cleavage sites for these enzymes.

CTSB and PRSS1 have compatible consensus sequences: (K/R)|X (where X is not Pro) for PRSS1, and (K/F)(K/R)|(Hy)(G/A) for CTSB, that were considered together to be a hybrid motif, similar to the design in FIG. 4 panel B. To avoid cleavages by the enzymes hepsin, thrombin or factor Xa, usage of Arg was avoided. The hybrid motif, therefore, comprises 28 unique sequences containing K or F in position M₂, K in position M₁, M, L, I, V, F, W or Y in position M₁′, and G or A in position M₂′.

The CTSB and lead tumor selective Gearr™ motifs are not compatible, therefore a tandem design is used to build a substrate sequence that contains both motifs, as shown in FIG. 4, panel B. One or more bracketing residues (position X₂) may be introduced between the two Gearr™ motifs. Bracketing residues are generally selected from small flexible amino acids such as Gly and Ser, but Ala and Pro can also be favorable. Thus, with the addition of a bracketing residue, four variants were required. For the configuration with two bracketing residue positions, this adds 16 variants. Finally, alternate conformations were also considered at the input hypothesis #2 stage, with CTSB either upstream or downstream of the tumor-selective Gearr™ motif, similar to the design in FIG. 4 panel B in which an alternate configuration GRLX₂PAX₁L can also accommodate all three Gearr™ motifs. If all variations are to be exhausted in this library design, the total number of variants include up to 6.8×10⁴ unique sequences. As the library scale increases with the addition of each variable element, information on the Experiment 3 refined subset of tumor selective Gearr™ motifs can be used to reduce the number of variations that are to be tested. In this case, it was further hypothesized that compatibility of the tumor-selective Gearr™ motif should be compatible with MMP9 cleavage. Thus Experiment 3 can be performed with MMP14 and MMP9 recombinant enzymes, and the resulting sequence variants of the tumor-selective Gearr™ motif can be prioritized to a short list of 4 candidates for incorporation into the sequence variants library designed with Algorithm 2. This would reduce the Algorithm 2 library size from 6.8×10⁴ to 3.584 unique substrate sequences.

In Step 4, the selectivity of these variant sequences is tested using many tissue biopsy samples across multiple diseased and healthy or non-diseased tissue types defined in Conditions #1, #2, and #3. In this setting, screening samples were chosen from tissues related to known adverse events that are linked to inhibition of the selected VEGF or VEGF receptor drug targets, such as placenta. In this case, for example, the cell line HTR-8/SVneo, a human immortal first-trimester EVT cell line representing normal placenta trophoblasts was selected. MMP14 and VEGF have significant roles in regulating angiogenesis in placenta (Chung et al., 2000; Kaitu'u-Lino et al., 2012), therefore placenta and pre-eclamptic placenta is an important Condition #2 and/or #3 tissue sample. Other normal tissues included in the Step 4 screening cohorts were: healthy breast tissue samples from mammaplasty, surgical biopsy samples of benign colorectal polyps or nodules and benign thyroid nodules, and similar samples from lung, liver, kidney, skin, ocular, pancreatic, ovarian, endometrial, urothelial, and cervical tissues. Selection of the number of samples for this screening process is influenced by principles of clinical design. Sufficient numbers of samples should be included to represent patient-to-patient variations. The end result is a panel of screening samples containing 50 to 100 samples.

Experiment 4 is a higher throughput format screening experiment. Therefore, the library of sequence variants designed with Algorithm 2 are built within a biological display screening system that can be chosen from phage, bacterial or mammalian expression systems. In this example, the Alauna™ method library is incorporated into the sequence of a surface-expressed scaffold protein from a mammalian cell line such as HEK293, CHO or HeLa. The library is designed on the protein level with amino acid sequences which specify the synthesis of oligonucleotide sequences that encode them. In this case, pools of oligonucleotide primers of length ˜120 nucleotides were designed using the principles of molecular cloning such as using Gibson cloning technology (Gibson et al., 2009) to assemble overlapping gene fragments and effectively insert the synthetic oligonucleotides encoding the Alauna™ method library into a mammalian surface expression platform system. Once the surface display system is built, the completeness of the library is tested using NGS genomic sequencing, by focusing the sequencing on the region containing the Alauna™ method library sequence insert. The goal for quality control of the system is that the library is 99.9% complete. In this case 1/1000 variants may be lost, effectively losing 4 clones out of the 3,584 that were designed in the library. Library completeness is an important feature of the Alauna™ method data analysis in Algorithm 3.

Conditioned media samples prepared from the set of selected tissues or cell lines representing Conditions #1, 2, and 3 are used as the source of peptidolytic activity, and a peptidase assay is performed using the pool of clones as the source of peptidase cleavable substrates. A pool of ten million mammalian cells can be suspended in a reaction volume of 1 milliliter for a peptidase assay. In this case, the clone library of 3,584 unique clones could be represented at >2,500-fold copy number within such a volume, to which is added the peptidase preparation at a fixed mass of total protein, such as 50 micrograms, from buffer exchanged conditioned media that has been filtered to remove any residual debris from the source tissues. Typical concentrations of peptidases in conditioned media were estimated at 0.5-1% peptidase by mass from the thyroid surgical samples utilized in Experiment 2.

The peptidolytic assay is performed under kinetic conditions, as in Experiments 1 and 2, such that during the course of the reaction, a fraction of the assay is withdrawn and the clones that have been cleaved can be distinguished by a dual color immunofluorescence assay such as may be performed using flow sorting techniques. For a one milliliter reaction of cells in suspension, the aliquots may be 200 microliter aliquots, collected at time points on the scale of minutes to hours. The resulting populations of sorted cells that were cleaved at each time point are then collected and prepared for NGS sequencing to identify the clones, and therefore the Alauna™ method substrate sequences that were cleaved at each time point. This preparation involves gentle centrifugation to collect the cells from suspension, leaving a supernatant that contains cleaved fragments from the substrate sequences. If further sequence optimization is to be performed, it is also important to determine the site of cleavage within these samples to help refine the input hypothesis #2. This analysis can be performed by analyzing the fragments that are released by the peptidase cleavage assay from the surface of the cells into the supernatant sample. Peptide sequencing by mass spectrometry is used for fragment identification using proteomic analysis; this process produces viable sequence information for top-most frequently detected substrates. In the end, the distribution of positions for where the scissile bond is found in each reaction will be informative about the elements that are most susceptible to peptidolytic cleavage in the designed substrate sequence: the tumor selective Gearr™ motif, the added CTSB/PRSS1 hybrid motif, the bracketing residues, and in which configuration these sites operate the most favorably. Kinetic ranking of the substrates within the library is informative about the anticipated rate of peptidolytic cleavage for the pro-drug activation under physiological or in vivo experimental conditions. The output #5 of this process includes a series of substrates with different catalytic rates or catalytic efficiencies of cleavage, obtained for all reactions with all 50-100 clinical samples representing Conditions #1, 2, and 3.

Algorithm 3 is a biostatistical analysis that is used to compare the performance of each substrate across the clinical samples, such as using a Welch's t-test for individual samples as well as sets of samples, re-defining Conditions #1, #2, and #3 in each comparison. For example, a first indication is papillary thyroid cancer. The selectivity of each substrate in the library is evaluated for the ability to be cleaved in all of the papillary thyroid cancer samples at a certain fold efficiency over all of the normal or healthy tissues tested. The specified fold efficiency that may be required is approximately 1:200, or 1:1000, or 1:10,000 in order to achieve the targeted therapeutic index for this pro-drug design.

The output #6 from this process is a ranked list of substrate sequences with the highest predicted level of tumor selectivity. Optionally, these sequences can be further evaluated in a time resolved Experiment #3, such as using a different platform for molecular detection of cleavage. One version of this experiment is to synthesize peptide substrate sequences and test their selective cleavage using recombinant MMP14, MMP9, CTSB and PRSS1, as well as selected tumor tissue samples. Alternatively, these substrate sequences can be inserted into reporter probes such as FRET probes or fluorescent labeling probes for in vitro or in vivo imaging applications.

The most selective substrate sequences that are obtained in Output #6 are then finally tested within a pro-drug or reporter molecule design. Such a design is diagrammed in FIG. 9, in which the simplest model is a domain A that interacts with the drug target, and a domain B that masks the domain A from interacting with the drug target. The selective peptidase cleavable substrate sequences that resulted from output #6 is then placed between domains A and B. For the detection of pro-drug activation, antibodies may be developed for an ELISA format assay to specifically recognize fragment 1 or fragment 2; these antibodies are useful molecules for evaluating cleavage within the Alauna™ method screening Experiment 4, as well as in subsequent mechanistic cell-based and pharmacokinetic analysis assays used to evaluate the pro-drug molecule.

In this embodiment, domain A is an anti-VEGF or anti VEGF receptor antibody, or a related molecule, and the domain B is a polypeptide that interferes with the binding of the antibody with its molecular drug target. The peptidase cleavable substrate is selected by the screening process outlined in Experiment 4.

### Example 2: Tissue- and Cell-Selective Cleavable Substrate Sequence Identification and Method of Use

The Alauna™ method was applied to the selection of therapeutic targets for the anti-inflammation indication.

Input hypothesis: (FIG. 1, Input #/1) Drug target selection: In one embodiment, the drug target comprises one or more targets selected from but not limited to the following: IL-1α, IL-1β, IL-5, IL-6, IL-8, IL-10, IL-17A, IL-23 p19, TNFα and TGFβ, IL-2R, IL-4R, IL-6R, IL-17R, a4B7 integrin, CD11a, CD74, CD163, and CD20. In one embodiment, the drug target comprises a surface antigen marker of leukocytes, including a487 integrin, CD11a, CD74, CD163, and CD20. In one aspect, an anti-inflammatory drug target is a cytokine or a cytokine receptor that participates in pro-inflammatory cytokine signaling. Examples of pro-inflammatory cytokines and cytokine receptors to be inhibited include but are not limited to, IL-1α, IL-1β, IL-5, IL-6, IL-8, IL-10, IL-17A, IL-23 p19, IL-33, IL-36α, IL-36β, IL-36γ, TNFα and TGFβ, IL-2R, IL-4R, IL-6R, IL-17R. In some embodiments, binding of the therapeutic agent to a pro-inflammatory cytokine or cytokine receptor, or to a surface antigen marker of leukocytes is dependent upon peptidase cleavage of a substrate sequence that releases a masking domain, which restricts binding of the therapeutic agent to the pro-inflammatory cytokine, cytokine receptor, or surface antigen marker of leukocytes only at the therapeutic site of action, as in the extracellular microenvironment of a leukocyte.

Peptidase target selection: The human genome encodes over 550 peptidases (Puente, 2003). Any of these peptidases can serve as target peptidases for pro-drug design, including several with previously developed peptidase cleavable substrate sequences (Table 1). Immune cells have well-known peptidase secretion functions. For example, activated neutrophils release peptidases from the serine protease family including neutrophil elastase, cathepsin G, serine protease 3, serine protease 4, as well as collagenase and gelatinase from the MMP family. The extracellular peptidases of neutrophils are involved in IL-1 family cytokine release and peptidase-catalyzed activation, which induces inflammation (Clancy et al., 2018). Induced macrophages release uPA and MMP9 (Pejler et al., 2003; Shapiro et al., 1991), and MMP14 is highly expressed in macrophages from patients with rheumatoid arthritis (Pap et al., 2000). Cytotoxic T lymphocytes and natural killer cells release granules containing granzymes, including GrA, GrB, GrH, GrK, and GrM with pro-inflammatory functions (Wensink et al., 2015). Thus, peptidase targets may be selected from those associated with leukocytes and induction of inflammation, including: neutrophil elastase, cathepsin G, serine protease 3, serine protease 4, MMP9 and other MMP family peptidases, uPA, and granzymes A, B, H, K. and M. The input hypothesis regarding peptidase selection for this embodiment was that one or more peptidases would serve as useful peptidase targets for multiple inflammatory conditions, such as rheumatoid arthritis, acute respiratory distress syndrome (ARDS), inflammatory bowel disease, chronic inflammatory lung, psoriasis, multiple sclerosis, pulmonary fibrosis, systemic lupus erythrematosis and other inflammatory indications. To narrow the selection of peptidases and drug target, Data Sets #1-4 as described in the Alauna™ method (FIG. 1, Input #1) were assembled to build a criteria matrix as follows:

Data Set #1, drug target and peptidase abundance: Gene expression data has been collected from inflammatory tissue samples such as synovial fluid from healthy individuals and patients with various forms of Rheumatoid arthritis (RA) (Platzer et al., 2019). Gene expression data is also available for inflammatory bowel disease (IBD), comparing IBD tissues (Condition #1) with tissues from uninvolved or normal colon (Condition #2 or #3), as well as using the CaCo2 cell line, which is used in GI tract studies (Dooley et al., 2004). The PulmonDB contains gene expression data for patients with Chronic Obstructive Pulmonary Disease (COPD) and Idiopathic Pulmonary Fibrosis (IPF) (Villaseñor-Altamirano et al., 2020). To prioritize selection of drug target and target peptidases for a rational approach, these data sets are queried for the correlation of the normalized gene expression levels for individual candidate drug targets (IL-1α, IL-1β, IL-5, IL-6, IL-8, IL-10, IL-17A, IL-23 p19, TNFα and TGFβ, IL-2R, IL-4R, IL-6R, IL-17R, a437 integrin, CD11a, CD74, CD163, and CD20) with all known human peptidases.

A therapeutic or diagnostic development program may opt to collect its own abundance Data Set #1, thereby defining Conditions #1, #2 and #3 with features related to disease phenotype and disease mechanism insights. For example, some patients with more severe Rheumatoid arthritis further develop extra-articular tissues related to tissue degradation, such as inflamed subcutaneous rheumatoid nodules. A recent analysis of the gene expression profiles from rheumatoid arthritis synovial fluid versus subcutaneous rheumatoid nodules revealed that while MMP1 and MMP3 were upregulated in the synovial fluid and in blood of RA patients, MMP7 and MMP12 (a macrophage elastase) were upregulated in the subcutaneous nodules (Kazantseva et al., 2013). This gives confidence to consideration of MMPs to be subject to tissue-specific upregulation, particularly in RA subcutaneous nodules. The goal for collecting Data Set #1 is to assess abundance either at the transcript or protein level for multiple peptidases, including MMPs, and to evaluate the correlation with the candidate drug target.

In one embodiment, an experimental plan to collect Data Set #1 for a tissue-selective analysis of RA was specified as follows. First, the therapeutic site defines the selection of tissue samples and the biofluids that bathe them in Condition #1. Synovial fluid samples from advanced stage RA (Condition #1) and similarly prepared samples from early stage RA (as a proxy for “normal” Condition #2 or #3) are one sample type. Another sample type is tissue biopsy from subcutaneous rheumatoid nodules (as a tissue-specific variation on Condition #1) and matched non-diseased adjacent tissues (Condition #2 or #3). These samples from Condition #1, #2, and #3 are selected for analysis of gene expression levels by RT-PCR and protein expression levels by ELISA. These samples will be tested for correlations between the set of candidate drug targets (IL-1a, IL-1β, IL-5, IL-6, IL-8, IL-10, IL-17A, IL-23 p19, TNFα and TGFβ, IL-2R, IL-4R, IL-6R, IL-17R, a437 integrin, CD11a, CD74, CD163, and CD20) and a set of selected peptidases including but not limited to: MMP family peptidases including MMP1, MMP3, MMP7. MMP9, MMP12, and MMP14, neutrophil elastase, cathepsin G, serine protease 3, serine protease 4, uPA, and granzymes A, B, H, K and M. This subset of protein targets comprises the key Data Set #1, but to also assure completeness, RNASeq transcriptomic analysis with NGS may be used to collect a full data set that includes all expressed peptidases as well as their inhibitors (Anamika et al., 2016).

The rational hypothesis in Input #1 (FIG. 3) further specifies that immune cells are contributing peptidase activities to the site of inflammation, with a set of known candidate peptidases that can serve as the peptidase targets for therapeutic design. In a further embodiment, Data Set #1 was also collected for a cell-selective analysis of leukocytes in RA. Leukocytes are isolated from whole blood from RA patients as well as from healthy patients, using principles of clinical design to select appropriate patient cohorts, preferably with a minimum of n=3-5 patients per cohort. The leukocytes from these donors can be isolated using an ammonium chloride treatment to remove RBCs, and then subjected to an exogenous activation treatment such as using phorbol myristate acetate to induce neutrophils, a strong T-cell antigen such as CD3/CD28 to activate T-cells, or LPS stimulation of monocytes, including macrophages. B cells. The conditioned media from these samples, preferably prepared serum-free or using synthetic serum (Jacking FBS), will then contain peptidases and inflammatory signaling molecules. These samples can be tested by ELISA or multiplexed immuno-assay to quantify levels of cytokines as a measure of inflammatory signaling. The procedures for lymphocyte isolation and stimulation are well developed (Stebbings et al., 2012). The abundance of peptidases will be tested by ELISA or directly in proteomic analysis as in Experiment 2 (Output #2). The conditioned media from the induced population of leukocytes from RA patients represents Condition #1, and from healthy patients they represent background Conditions #2 and/or #3.

A corollary to the hypothesis in Input #1 is that the identity of peptidases from the population of leukocytes in RA patients versus those in healthy patient leukocytes may be the same, but their relative levels at the protein and peptidase activity level will differ between individuals corresponding to disease severity. These conditioned media samples also represent the extracellular milieu to be targeted as the therapeutic site of action for the development of cell-selective peptidase cleavable substrate sequences. The analysis of Data Set #1 for leukocytes is used to narrow the list of peptidase targets in the leukocyte milieu that are released by RA versus healthy donor cells. The peptidases that most correlate with markers of inflammation are selected as target peptidases.

Proteomic (histopathological) and transcriptomic analyses of normal tissues in the Human Protein Atlas (version 18.1, release date 2018 Nov. 15) (Fagerberg et al., 2014; Uhlén et al., 2015) reveal additional information on broad expression profiles of the set of candidate peptidases. Peptidases such as neutrophil elastase, cathepsin G and the granzymes have tissue specific expression in bone marrow and lymphatic tissues. The MMPs have broad expression profiles, and therefore their activity would be contributed by the inflamed tissue at the therapeutic site of action. Condition #1 would ideally be a condition where multiple peptidase activities are upregulated, and Condition #3 would be the normal healthy tissues that express one or more of these peptidases. Among multiple MMPs considered during hypothesis refinement, the Human Protein Atlas data showed that MMP14 is broadly expressed across multiple tissue types at low levels, including the GI tract and female tissues, and MMP7 has elevated expression in kidney and salivary gland. Neutrophil elastase is expressed in bone marrow and lymph tissues, and granzyme B is expressed in lymphoid tissues.

Data Set #2, peptidase substrate information. At the outset of the project, one criterion for selection of a target peptidase or family of peptidases is the probability that a new peptidase cleavable substrate sequence can be identified that will meet requirements for disease-selective activation. The Alauna™ method does not strictly require a rational hypothesis that selects a peptidase target at the start of a project. Thus, consideration of Data Set #2 is optional, but information on the specificity of a targeted peptidase can be added to the substrate refinement process (FIG. 3, Step 3), and when a peptidase target is likely to be highly specific, this adds confidence that the process will yield the specified disease-selective activation required for a therapeutic application.

In this embodiment, Data set #2 included substrate sequence information from the MEROPS database for MMP14 as well as other MMPs (Rawlings, 2016; Rawlings et al., 2004). A consensus sequence of PXG|L, where the “|” indicates the scissile bond, was found for MMP14 in the MEROPS database, although the source studies for this data were not readily available in the database. Biological display methods have been reported elsewhere for MMP14 (Jabaiah and Daugherty, 2011; Kridel et al., 2002), as well as other candidate substrate library approaches (Turk et al., 2001), which, although each approach suffers from some limitations or sequence bias resulting in different consensus sequences, each showed amino acid preferences in at least three sites within the substrate sequence, indicating a probability that specific and selective substrates could be obtained for MMP14 among other MMPs. Other MMPs also cleave a similar PXG|L consensus sequence, but other sites outside of these positions have been shown to contribute to specific cleavage with MMP14 vs other peptidases, e.g. (Turk et al., 2001) that suggest that further MMP specificity can be obtained.

Further investigation of the specificity data available in MEROPS for the leukocyte peptidases shows that each candidate has a balanced substrate specificity profile that is dependent upon multiple substrate recognition sites. The candidate Gearr™ motifs for neutrophil elastase will depend upon positions M₃-M₁, based on the amino acid frequencies summarized by the consensus sequence (E/Q)(P/G)(V/I)|. For granzyme B, the Gearr™ motif depends upon positions M₄-M₁, based on the consensus sequence IE(G/P/A/V/L)D|; and for uPA it positions M₃-M₁, represented by the consensus SGR|. The latter is incorporated into current cleavable linker sequences in Table 1. Given its dependence upon the basic residue arginine, uPA is less favored as a candidate due to the potential for cleavage by background serine peptidases in circulation, many of which catalyze cleavage at Arg.

Data Set #3 included enzyme activity data aggregated from the BRENDA database (accessed November 2019) for MMP14, MMP7, granzyme B, neutrophil elastase and uPA.

The BRENDA database lists several natural substrates for MMP14, such as collagen type I alpha chain and pro-MMP2, consistent with its role in promoting degradation of the extracellular matrix and in activating other MMPs. Typical kinetic parameters for MMP14, measured with tool substrates, were listed with low nanomolar Michaelis constants (K_{M}), and picomolar K_{M} values were measured for collagen I alpha-1 and -2 chain substrates. The fluorescent probe methoxycoumarin-4-acetyl-Lys-Pro-Leu-Gly-Leu-Lys (2,4-dinitrophenyl)-Ala-Arg-NH₂ had a reported turnover number of 0.33-6.8 (s⁻¹), and catalytic efficiency values of 550-2429 mM⁻¹ s⁻¹ measured by fluorescence assay. All of these values are comparable to MMP9 values, measured toward similar substrates as reported in the BRENDA database.

Substrates for granzyme B include aggrecan and cartilage proteoglycans, both relevant to RA, and several other extracellular substrates (Boivin et al, 2009). The top-most active substrates reported in the BRENDA database such as the fluorescent tool substrate 2-aminobenzoyl-IEPDSSMESK-dnp, have micromolar Michaelis constants (K_{M}), and turnover numbers (k_{cat}) of 1-5 s⁻¹, which translates to an estimated catalytic efficiency of 758 mM⁻¹ s⁻¹ measured by fluorescence assay (Sun et al., 2001). Candidate natural substrates such as beta-glycan, biglycan and decorin have measured catalytic efficiencies of 5.9, 1.7 and 1 mM⁻¹ s⁻¹, respectively, as measured by fluorescence assay (Boivin et al., 2012).

A similar analysis of the BRENDA database information for neutrophil elastase is rich with information. Natural substrates reported in this database include: IL1, IL2, IL6, cartilage proteoglycan, CD2, CD4, CD8, collagens type I, II, III, IV, collagenase, gelatinase, MMP7, MMP9, progranulin, plasminogen, and several others. The top-most active substrates reported in the BRENDA database such as the fluorescent tool substrate methoxysuccinyl-Ala-Ala-Pro-Val-thiobenzyl ester, have micromolar Michaelis constants (K_{M}), and turnover numbers (k_{cat}) of up to 22 s⁻¹, which translates to an estimated catalytic efficiency of 9565 mM⁻¹ s⁻¹ measured by fluorescence assay (Stein et al., 1987).

Based on the above analysis of the Data Sets #2 and #3 from the MEROPS and BRENDA databases, each of the candidate peptidases: MMPs, neutrophil elastase, and granzyme B, qualify on the level of activity required for the specification of a pro-drug activation, with several highly relevant endogenous substrates that support a mechanistic involvement of these peptidases in the diseased tissues of RA.

Data Set #4, subcellular localization: MMP14, as a member of the membrane-type matrix metalloproteases (MT-MMP1), is localized to the cell surface. This membrane localization is attractive for application to tumor-targeting pro-drug design as this would predict more restricted localization than secreted forms of MMPs and other peptidases. MMP14 also has a sheddase function that releases proteins from the cell surface, similar to the A Disintegrin and metalloproteinase domain-containing (ADAM) family of peptidases; known sheddase substrates of MMP14 include EMMPRIN, kidney injury molecule I, MHC class I chain-related molecule A, and other regulated proteins (Egawa et al., 2006; Guo et al., 2012; Liu et al., 2010).

As described above, neutrophil elastase, cathepsin G, serine protease 3, serine protease 4, uPA, and granzymes A, B, H, K and M are all released by stimulated leukocytes, including neutrophils, macrophages, NK cells, dendritic cells, B cells and T cells. Thus, they are localized to the extracellular microenvironment and can be used for activation of a prodrug that targets a secreted protein or surface-bound receptor.

Selection of Conditions #1, #2, and #3: As described in collection of Data Set #1, disease related samples are required to represent Conditions #1, #2, and #3. In the category of tissue-specific samples, these include: Synovial fluid samples from advanced stage RA (Condition #1) and similarly prepared samples from early stage RA (as a proxy for “normal” Condition #2 or #3), as well as tissue biopsy from subcutaneous rheumatoid nodules (as a tissue-specific variation on Condition #1) and matched non-diseased adjacent tissues (Condition #2 or #3). In the category of cell-specific samples, these include conditioned media prepared from leukocytes isolated from the whole blood of RA patients (representing Condition #1) as well as from healthy patients (representing Conditions #2 and #3).

The anti-IL6 and anti-IL6 receptor monoclonal antibody therapies tocilizumab and sarilumab are in use for rheumatoid arthritis. The most frequently reported adverse events with these agents are infections including UTI and upper respiratory tract infections. Patients can also have neutropenia, as well as elevated serum cholesterol levels and increased liver enzymes (Hennigan and Kavanaugh, 2008; McCarty and Robinson, 2018). Review of the human protein atlas tissue distribution data shows IL6 expression in the lung, kidney, urinary, bladder and bone marrow. Thus, sampling of healthy primary tissues for Conditions #2 and #3 should include: hepatic, lung, kidney, urinary and bladder tissues. One skilled in the art will also select tissue samples or cell models that match cell-based assays used in mechanistic studies to verify efficacy as part of the therapeutic or diagnostic development program.

Broad screening across patients is introduced at Step 4 of the Alauna™ method. The screening should be performed with synovial fluid samples from advanced stage RA, early stage RA (as a proxy for “normal”), conditioned media prepared with tissue biopsy from subcutaneous rheumatoid nodules and matched non-diseased adjacent tissues, conditioned media prepared from leukocytes from donors with RA vs healthy patients, and conditioned media prepared from selected tissues with connection to any known adverse events in this area of anti-inflammatory therapeutics. These will total 50-100 primary samples.

To supplement Data Sets #1 and #4, histology data for tissue co-localization of candidate peptidases such as MMP14, MMP7, neutrophil elastase, and granzyme B with the candidate drug targets such as IL6 and IL6 receptor proteins, is used to prioritize the candidate tissues with the highest likelihood of co-localization. Tissues from this list that are negative for extracellular peptidase represent Condition #2; tissues that are positive for peptidases but negative for IL6 or IL6 receptors represent Condition #3. In addition, to assess stability of a candidate MMP14, MMP7, neutrophil elastase, and granzyme B cleavable substrate sequence, counter screening for Condition #3 can include samples prepared from healthy compartments such as circulatory peptidases in serum or plasma (Jambunathan and Galande, 2014).

Discovery Experiments, Alauna™ method Step 2 (FIG. 3).

In this embodiment, the target peptidase or set of peptidases was selected from the family of MMP enzymes, specifically MMP14 and MMP7, optionally with or without neutrophil elastase and/or granzyme B.

Experiment 1 is a multiplexed substrate profiling assay performed using purified recombinant enzymes. A suitable Alauna™ method unbiased library was selected by matching to the required level of complexity for the application. In this embodiment, the MMP family of peptidases, and specifically MMP14 and MMP7 have been selected as the primary peptidase target(s). The MMP family of peptidases share consensus sequences that can be summarized as PX(Sm)|(Hy), where “Sm” are small residues such as G, A, S, N, E and “Hy” are hydrophobic residues such as L, M, Y, I, F (Turk et al., 2001).

As described in Example 2, it is an ongoing challenge to identify highly “specific” substrates, meaning substrates that are cleaved more rapidly, more efficiently, or more completely, than other substrates by an individual MMP family member. It is similarly difficult to identify, highly “selective” substrates, meaning substrates that is more rapidly, more efficiently, or more completely cleaved by one MMP versus another member of the MMP family using existing technologies. This is because all existing technologies essentially collapse the information collected from a set of profiled peptidase substrates into consensus motifs that summarize amino acid frequency at each position in the sequence, ignoring the cooperativity, i.e. the connectivity, between amino acids within those motifs. Nevertheless, the data collected to date for the MMP family indicates that the individual MMPs require at least three positions to define a substrate that can be recognized and cleaved (Turk et al., 2001).

To determine the specificity of individual peptidases from the MMP family, the Alauna™ method unbiased library 2.4 was used in a multiplexed substrate profiling assay performed in Experiment 1 with MMP14 and MMP9 as a proxy for other MMP family peptidases. For this experiment, recombinant enzymes (R&D Systems, #911-MP and #918-MP) were activated according to manufacturer's recommendations. The enzymes were assayed at pH 7.4 in 50 mM TRIS HCl buffer containing 1 mM CaCl₂, at low nanomolar concentrations, typically 10 nM, to catalyze cleavage of synthetic peptides within the Alauna™ method unbiased library 2.4. The synthetic peptides that compose the Alauna™ method unbiased library 2.4 were prepared by standard solid-phase peptide synthesis, and they were combined into a single pool of substrates, prepared at 500 nM equimolar concentration for each substrate in the peptidase reaction. The peptidase assays were performed in a kinetic format, removing aliquots and quenching the reaction with 1% formic acid at multiple time points over the course of the reaction, ranging from minutes to hours. These aliquots were immediately desalted using C18 desalting tips and standard mass spectrometry compatible solvents (Solvent A: 0.1% formic acid in HPLC grade water, Solvent B: 50% acetonitrile, 0.1% formic acid in water), and then dried under a vacuum prior to analysis by LC-MS/MS. Thus, for data analysis it is possible to apply enzyme kinetics principles to assess the observed rate of cleavage (k_{obs}), catalytic efficiency (K_{cat}/K_{M}), and maximum yield of substrate cleavage (% Substrate consumption or % Product formation). In this case, a plateau in maximum product formation (% P) was used as a threshold for binary determination of whether or not a unique bond cleavage occurred after a fixed reaction time. The raw data output of this analysis, (Output #1, FIG. 3) is a list of cleaved sequences aligned by their scissile bond.

In this embodiment, the Alauna™ method unbiased library 2.4 was sufficient to identify Gearr™ motifs composed of more than three amino acid positions for the MMPs by solving for pairs of cooperative interactions that were enriched among the cleaved substrates compared to the total library distribution of possible pairwise interactions. Shown in FIG. 7 are the sequence logo representations of the results of this experiment for MMP14 and for MMP9. In the Experiment 1 for MMP14, there were 80 observed unique substrate cleavages within the Alauna™ method unbiased library 2.4, as measured with mass spectrometry. Among these cleavages were four pairs of cooperative residues that were enriched as compared to the background library sequences, indicating favorable cleavages. All of these cooperative interactions can be summarized with the Gearr™ motif PXG(M/L/I)Y for MMP14. In the experiment with MMP9, 241 unique cleavages were observed, with six pairs of cooperative interacting residues that were enriched at least 2-fold over background, and that resulted in a Gearr™ motif of PG(S/G)(I/R/M/L)XS for MMP9 (FIG. 7). Preferably the same Experiment 1 format and analysis is also performed with MMP7 to identify any unique cooperative interactions among cleaved substrates for this enzyme. Any unique cooperative interactions may be used to enhance the selectivity of the output Gearr™ motif for MMP7 and MMP14 over other MMPs such as MMP9.

The result of Experiment 1 is a candidate Gearr™ motif that summarizes all of the cooperative interactions observed at a certain threshold, in this case >2-fold enrichment over background, that were measured for a peptidase activity experiment under specified conditions. These Experiment 1 results clearly demonstrated that a Gearr™ motif is distinct from a standard consensus sequence or consensus motif obtained with standard methodology in the peptidase field. A Gearr™ motif captures the connectivity between residues as a co-association that is described as cooperativity, whereas a consensus sequence aims to summarize the amino acid frequency observed at all positions within a substrate sequence, ignoring their connectivity.

Experiment 2 is a biological discovery experiment performed using patient-derived as well as cell-based samples, representing Conditions #1, #2 and #3 as defined by the Input hypothesis (FIG. 3, Input #1).

The first step of Experiment 2 is the preparation of conditioned media samples with flash-frozen surgical biopsy samples of approximately 100 mg tissue mass. Tissue samples from a first series of discovery screens include pathologically-graded rheumatoid arthritis synovial fluid samples from advanced stage RA (Condition #1), and early stage RA (as a proxy for “normal”, Condition #2 and #3), conditioned media prepared with tissue biopsy from subcutaneous rheumatoid nodules (Condition #1) and matched non-diseased adjacent tissues (Condition #2 and #3), and conditioned media prepared from leukocytes from donors with RA (Condition #1) vs healthy patients (Condition #2 and #3). To test other types of normal healthy tissues, primary tissues and immortalized “healthy” cell lines are used, such as may be collected from colon nodule biopsy (graded benign), as well as human lung, kidney, or bladder epithelial cells.

For Experiment 2, conditioned media are prepared from tissue that has been thawed and rinsed twice with phosphate buffered saline (PBS) to remove any visible blood from the sample, prior to dicing them into approximately 1 mm³ pieces. These are rinsed in a warm basal cell culture medium such as Dulbecco's Modified Eagle's medium (DMEM) without supplemented FBS, which can contain factors that alter the peptidase profiles of the samples, and then the media is replaced with a typical volume of 1:30, tissue wet weight to medium volume. The tissue is incubated in the medium under standard mammalian cell culture conditions for 16 hours, then the conditioned medium is collected and replaced with fresh medium for another 16 hours, and combined with the first to produce neat conditioned medium. The tissue may be saved for processing into a cell lysate as well, in case additional markers are to be tested with immunodetection or proteomic analysis. Cytotoxicity is routinely tested in these samples by the lactose dehydrogenase assay, aiming to maintain the samples in culture for up to 3 days, as long as cell death remains <5%. The conditioned medium is then buffer exchanged and concentrated with cell culture-grade PBS. The final total protein concentration is determined in the conditioned medium by BCA assay, and typically is in the range of 1-5 mg/ml.

For synovial fluid, the samples may be used directly in any proteomic and peptidase activity screening for Experiment 2. The samples are collected and immediately flash frozen for storage prior to biochemical analysis. The concentration of total protein is determined for synovial fluid and conditioned media samples by protein bicinchronic acid (BCA) assay.

These conditioned media and synovial fluid samples represent the input for Experiment 2. For proteomic analysis, approximately 10 micrograms of total protein from the buffer-exchanged, conditioned medium are digested with sequencing grade trypsin and subjected to semi-quantitative, label-free proteomic analysis using liquid-chromatography tandem mass spectrometry (LC-MS/MS). Proteomic data are output with spectral counts as an approximation of relative protein abundance. Peptide counts are normalized by the total counts from a single MS sample. Samples are analyzed individually, and the resulting protein identifications are output with spectral counts as an approximation of relative protein abundance. To correct for sample loading differences, the spectral count measurements are normalized to total signal in the sample, missing values were imputed, and the normalized spectral count data are log₂-normalized for ratiometric comparison between samples. Individual comparisons can be made between samples, as well as between sets from Condition #I versus Condition #2 or #3 using a Welch's t-test in the R statistical computing environment. The peptidases identified in the individual samples inform selection of additional peptidases for Experiment #1. These data also are used to supplement Data Set #1 (FIG. 3, Input #1). Any additional peptidases identified that are enriched in Condition #1 vs Condition #3, such as additional granzyme or MMP family members or other serine peptidases specified in Input #1 can be included as appropriate.

Currently, the leading candidates for additional peptidase activities are granzyme B and neutrophil elastase. Accordingly, granzyme B and neutrophil elastase are selected for Experiment 1 substrate profiling with the Alauna™ method unbiased library 2.4, using the same approach as was used for the MMPs. These enzymes are activated according to the manufacturer recommendations, and tested using the Alauna™ method with assay conditions matching the pH 7.4 reactions for MMP14 and MMP9. The output Gearr™ motifs resemble the simple consensus sequences for neutrophil elastase: (E/Q)(PG)(V/I)| in positions M₃-M₁ and for granzyme B: IE(G/P/A/V/L)D| in positions M₄-M₁. Thus, the specificity of these two enzymes are incompatible for a hybrid cleavage motif design, and are instead built into a tandem motif design using Algorithm 2.

Peptidase activity profiling in Experiment 2 (Output #3) is performed using buffer-exchanged conditioned media or synovial fluids from the same set of Condition #1, #2 and #3 samples as were used for proteomic discovery (Output #2), to investigate differential activities observed in severe versus early stage RA vs healthy donor samples, using the Alauna™ method unbiased library 2.4. Since the enzyme titer in each sample is unknown, the reactions are normalized by using a fixed total amount by mass of protein from the conditioned media samples as the peptidase sample, and the reactions are performed in a kinetic format to assess enzyme-catalyzed cleavage. As in Experiment 1, a plateau in maximum product formation (% P) is used as a threshold for binary determination of whether or not a unique bond cleavage occurred after a fixed reaction time in each sample. The resulting cleavages within this library from each reaction in Output #3 are filtered for endo-peptidolytic cleavages that were strictly observed in the severe RA conditioned media or synovial fluid samples as compared to the early RA, non-diseased adjacent tissue, or healthy donor samples. The raw data output of this analysis, (Output #3, FIG. 3) is a list of cleaved sequences aligned by their scissile bond from each Condition #1, #2, and #3.

Since these samples represent multiple pairs of matched samples, the opportunity arises to apportion out the enriched cleavages into unique Gearr™ motifs that are tissue- and/or cell-selective. Specifically, one embodiment of Output #4 of Algorithm #1 is the tissue-selective Gearr™ motif that differentiates by disease severity between severe RA and early stage RA donors, as produced by the matched synovial fluid samples. Alternatively, a second embodiment of Output #4 from Algorithm #1 is the tissue-selective Geary™ motif that differentiates between subcutaneous rheumatoid nodules and matched non-diseased tissues. Yet another embodiment of Output #4 from Algorithm #1 is the cell-selective Gearr™ motif that differentiates between the peptidase activities of activated leukocytes from RA versus healthy donors. Thus, three candidate RA-selective Gearr™ motifs result from Algorithm 1 that can be further tested in Steps 3 and 4 of the Alauna™ method. These Gearr™ motifs may be used for different drug target applications, and should therefore be carried forward into the Step 3 design process.

In this embodiment, since IL6 is a secreted protein and IL6R is a cell surface accessible protein, the stability of an anti-IL6 or anti-IL6R pro-drug in circulation is an important pharmacokinetic feature, thus susceptibility of the substrate sequence to cleavage by non-targeted peptidases in healthy tissues or compartments, such as the circulating enzymes factor Xa and thrombin, can be considered to be active peptidases from Condition #3. Thrombin has a consensus motif of (G/P)(R/K)| and Factor Xa has a slightly more broad consensus motif of (P/G/A/L)(R/K)|S. The Gearr™ motif for these enzymes, when assayed in Experiment 1 with the Alauna™ method unbiased library 2.4, will be used to identify cooperative pairs of residues with the three candidate RA-selective Gearr™ motifs.

In this embodiment, the main application of Algorithm 1 is to perform differential analysis of sets of cleaved substrate sequences obtained from Experiments 1 and 2, Output #1 and #3. The conditions defined by the Input #1 help to define which sets of cleavages are to be compared. In order to ascertain whether the non-diseased tissues qualify as negative for presence of the drug target (representing Condition #2) or for presence of the target peptidase (Condition #3), the relative abundance of these proteins is determined using immuno-detection or proteomic analysis at the protein level. The activity for MMPs in general can also be quantified and compared between samples using highly sensitive enzymatic assays such as by using the fluorescent substrate methoxycoumarin-4-acetyl-Lys-Pro-Leu-Gly-Leu-Lys (2,4-dinitrophenyl)-Ala-Arg-NH₂ to detect MMP activity. The generic substrates MEOSUC-Ala-Ala-Pro-Val-AMC or t-Butyloxycaronyl-Ala-Ala-Asp-ThioBenzyl ester can be used to test for neutrophil elastase-like or granzyme B-like activity as well. While these substrate probes are not ideally specific for each of these enzymes, the absence of activity would add to the confidence of assigning samples to Condition #3.

Output #4 from Algorithm 1 is a set of three Gearr™ motifs, comprising multiple derivative sequences that have a range of kinetic rates and overall yields of cleavage. Since the patient-derived samples contain multiple peptidases, a single variation within a substrate sequence can affect the kinetics of all peptidases that recognize the Gearr™ motif within it. Therefore, lead candidate substrate sequences should be empirically tested to verify and quantify cleavage. In this embodiment, catalytic efficiency is measured because it allows comparison of selectivity for cleavage between individual samples and conditions. In Step 3, all possible variants are calculated for each candidate RA-selective Gearr™ motif, which will likely include hundreds of variants.

In order to reduce the complexity of these variants for the RA-selective Gearr™ motif, a small library is prepared for Experiment 3 prior to starting the Step 3 process of new library design with Algorithm 2. In Experiment 3, the catalytic efficiency of cleavage for the three candidate RA-selective Gearr™ motifs plus a small number of conservative variants are tested to prioritize a smaller number of lead candidate substrate sequences. In this embodiment, conservative variants include one or more residue substitutions, made using the observed cooperative residues identified in the second or third motif. These substitutions produce sequence variants that share greater similarity with each other; Experiment #3 is used to validate that a candidate RA-selective Gearr™ motif will be favorably cleaved by each of the targeted peptidases, and secondarily to test which cooperative residue pairs can be added to each motif to build a more unified candidate RA-selective Gearr™ motif that can function for RA-selective cleavage.

The peptidases that are ultimately selected for Step 3 of the Alauna™ method are chosen based on all evidence available. If by proteomic analysis, additional peptidases are identified in Condition #1, these can be considered in the criteria matrix evaluation process. In one embodiment, the targeted peptidases include MMP14, MMP7, granzyme B and neutrophil elastase. In another embodiment the targeted peptidases may include one or two of these selected peptidases. Using the consensus motifs for these enzymes identified from Experiment 1 with the Alauna™ method unbiased library 2.4, it was decided at Input #2 to test whether addition of these substrate cleavage sites could enhance the rate or efficiency of cleavage for the candidate substrates. Therefore, configurations were designed using Algorithm 2 that also incorporate candidate cleavage sites for these enzymes.

In one embodiment, none of the three selected peptidases had compatible consensus sequences with any of the candidate RA-selective Gearr™ motifs. The consensus sequence from external data in the MEROPS database is (E/Q)(P/G)(V/I)| for neutrophil elastase, and for granzyme B it is IE(G/P/A/V/L)D|. Thus, an arrangement of the candidate RA-selective selective Gearr™ motif, neutrophil elastase and granzyme B can take six tandem arrangements to test the effects of motif order: A-B-C, A-C-B, B-A-C, B-C-A, C-A-B. and C-B-A. Alternately, only two peptidases may be selected, and the number of configurations is still six: A-B, B-A, A-C, C-A, B-C, C-B.

When Gearr™ motifs are not compatible, a tandem design is used to build a substrate sequence that contains each selected motif. One or more bracketing residues, typically up to four, may be introduced between the Gearr™ motifs. Bracketing residues are generally selected from small flexible amino acids such as Gly and Ser, but Ala and Pro can also be favorable. If all variations of just the arrangement and bracketing residue spacing are to be tested in this library design, the total number of variants include up to 6.3×10⁴ unique sequences. As the library scale increases with the addition of each variable element, information on the Experiment 3 refined subset of tumor selective Gearr™ motifs can be used to reduce the number of variations that are to be tested.

In Step 4, the selectivity of these variant sequences is tested using many tissue biopsy samples across multiple diseased and healthy or non-diseased tissue types defined in Conditions #1, #2, and #3. Selection of the number of samples for this screening process is influenced by principles of clinical design. Sufficient numbers of samples should be included to represent patient-to-patient variations.

Experiment 4 is a higher throughput format screening experiment. Therefore, the library of sequence variants designed with Algorithm 2 are built within a biological display screening system that can be chosen from phage, bacterial or mammalian expression systems. In this example, the Alauna™ method library is incorporated into the sequence of a surface-expressed scaffold protein from a mammalian cell line such as HEK293. CHO or Hela. The library is designed on the protein level with amino acid sequences which specify the synthesis of oligonucleotide sequences that encode them. In this case, pools of oligonucleotide primers of length ˜120 nucleotides were designed using the principles of molecular cloning such as using Gibson cloning technology (Gibson et al., 2009) to assemble overlapping gene fragments and effectively insert the synthetic oligonucleotides encoding the Alauna™ method library into a mammalian surface expression platform system. Once the surface display system is built, the completeness of the library is tested using NGS genomic sequencing, by focusing the sequencing on the region containing the Alauna™ method library sequence insert. The goal for quality control of the system is that the library is 99.9% complete. Library completeness is an important feature of the Alauna™ method data analysis in Algorithm 3.

Conditioned media samples prepared from the set of selected tissues or cell lines representing Conditions #1, 2, and 3 are used as the source of peptidolytic activity, and a peptidase assay is performed using the pool of clones as the source of peptidase cleavable substrates. A pool of ten million mammalian cells can be suspended in a reaction volume of 1 milliliter for a peptidase assay. In this case, the clone library of 6.3×10⁴ unique clones could be represented at >150-fold copy number within such a volume, to which is added the peptidase preparation at a fixed mass of total protein, such as 50 micrograms, from buffer exchanged conditioned media or neat synovial fluid that has been filtered to remove any residual debris from the source tissues. Typical concentrations of peptidases in conditioned media were estimated at 0.5-1% peptidase by mass based on typical samples utilized in Experiment 2.

The peptidolytic assay is performed under kinetic conditions, as in Experiments 1 and 2, such that during the course of the reaction, a fraction of the assay is withdrawn and the clones that have been cleaved can be distinguished by a dual color immunofluorescence assay such as may be performed using flow sorting techniques. For a one milliliter reaction of cells in suspension, the aliquots may be 200 microliter aliquots, collected at time points on the scale of minutes to hours. The resulting populations of sorted cells that were cleaved at each time point are then collected and prepared for NGS sequencing to identify the clones, and therefore the Alauna™ method substrate sequences that were cleaved at each time point. This preparation involves gentle centrifugation to collect the cells from suspension, leaving a supernatant that contains cleaved fragments from the substrate sequences. If further sequence optimization is to be performed, it is also important to determine the site of cleavage within these samples to help refine the input hypothesis #2. This analysis can be performed by analyzing the fragments that are released by the peptidase cleavage assay from the surface of the cells into the supernatant sample. Peptide sequencing by mass spectrometry is used for fragment identification using proteomic analysis; this process produces viable sequence information for top-most frequently detected substrates. In the end, the distribution of positions for where the scissile bond is found in each reaction will be informative about the elements that are most susceptible to peptidolytic cleavage in the designed substrate sequence: the RA selective Gearr™ motif, the motifs for neutrophil elastase or granzyme B, the bracketing residues, and in which configuration these sites operate the most favorably. Kinetic ranking of the substrates within the library is informative about the anticipated rate of peptidolytic cleavage for the pro-drug activation under physiological or in vivo experimental conditions. The output #S of this process includes a series of substrates with different catalytic rates or catalytic efficiencies of cleavage, obtained for all reactions with all 50-100 clinical samples representing Conditions #1, 2, and 3.

Algorithm 3 is a biostatistical analysis that is used to compare the performance of each substrate across the clinical samples, such as using a Welch's t-test for individual samples as well as sets of samples, re-defining Conditions #1, #2, and #3 in each comparison. For example, a first indication is RA. The selectivity of each substrate in the library is evaluated for the ability to be cleaved in all of the RA samples at a certain fold efficiency over all of the non-diseased or healthy tissues tested. The specified fold efficiency that may be required is approximately 1:200, or 1:1000, or 1:10,000 in order to achieve the targeted therapeutic index for this pro-drug design.

The output #6 from this process is a ranked list of substrate sequences with the highest predicted level of RA selectivity. Optionally, these sequences can be further evaluated in a time resolved Experiment #3, such as using a different platform for molecular detection of cleavage. One version of this experiment is to synthesize peptide substrate sequences and test their selective cleavage using recombinant MMP14, MMP7, granzyme B and neutrophil elastase, as well as selected RA tissue samples.

The most selective substrate sequences that are obtained in Output #6 are then finally tested within a pro-drug or reporter molecule design. Such a design is diagrammed in FIG. 9, in which the simplest model is a domain A that interacts with the drug target, and a domain B that masks the domain A from interacting with the drug target. The selective peptidase cleavable substrate sequences that resulted from output #6 is then placed between domains A and B. For the detection of pro-drug activation, antibodies may be developed for an ELISA format assay to specifically recognize fragment 1 or fragment 2; these antibodies are useful molecules for evaluating cleavage within the Alauna™ method screening Experiment 4, as well as in subsequent mechanistic cell-based and pharmacokinetic analysis assays used to evaluate the pro-drug molecule.

In this embodiment, domain A is an anti-IL6 or anti IL6 receptor antibody, or a related molecule, and the domain B is a polypeptide that interferes with the binding of the antibody with its molecular drug target. The peptidase cleavable substrate is selected by the screening process outlined in Experiment 4.

### INCORPORATION BY REFERENCE

The entire disclosures of all patent and non-patent publications cited herein are each incorporated by reference in their entireties for all purposes.

### OTHER EMBODIMENTS

The disclosure set forth above may encompass multiple distinct disclosures with independent utility. Although each of these disclosures has been disclosed in its preferred form(s), the specific embodiments thereof as disclosed and illustrated herein are not to be considered in a limiting sense, because numerous variations are possible. The subject matter of the disclosures includes all novel and nonobvious combinations and subcombinations of the various elements, features, functions, and/or properties disclosed herein. The following claims particularly point out certain combinations and subcombinations regarded as novel and nonobvious. Disclosures embodied in other combinations and subcombinations of features, functions, elements, and/or properties may be claimed in this application, in applications claiming priority from this application, or in related applications. Such claims, whether directed to a different disclosure or to the same disclosure, and whether broader, narrower, equal, or different in scope in comparison to the original claims, also are regarded as included within the subject matter of the disclosures of the present disclosure.

1. A method of constructing an unbiased library of peptides or proteins, comprising:
i. designing a peptide library by a combination of biological assays and computational modeling wherein each peptide comprises at least a set of two, three or more amino acid motifs;
ii. generating a plurality of peptides comprising the at least a set of two, three or more amino acid motifs which are variable based on amino acid sequences and positioning of each motif in each peptide;
iii. assessing cooperative interactions between at least two, three or more amino acid motifs within a given sequence space for a defined number of amino acid residues and length of a peptide substrate sequence;
iv. extracting of amino acid motifs by a computational algorithm by measuring cooperativity between two or more unique amino acids found at specific positions within substrate sequences of a peptide library;
thereby constructing an unbiased library of peptides or proteins.

2. The method of claim 1, wherein the at least two, three or more amino acid motifs are extracted by a computational algorithm comprising measuring cooperativity between two or more unique amino acids found at specific positions within substrate sequences of a library.

3. The method of claim 2, wherein the at least two, three or more amino acid motifs comprise a minimal set of two or more amino acid positions within a substrate sequence that interacts with one or more enzymes.

4. The method of claim 3, wherein the amino acids comprised in a particular amino acid motif have a positive or negative effect upon the rate of scissile bond cleavage by the one or more enzymes.

5. The method of claim 3, wherein the substrate sequence is an enzyme substrate sequence capable of being cleaved by at least one enzyme.

6. The method of claim 1, wherein the at least one enzyme comprises one or more peptidases.

7. The method of claim 6, wherein the one or more peptidases comprise: endopeptidases, omega-peptidases, exopeptidases, aminopeptidases, carboxypeptidases, dipeptidyl-peptidases, tripeptidyl peptidases, peptidyl dipeptidases, dipeptidases, or combinations thereof.

8. The method of claim 7, wherein the unbiased library of proteins or peptides is incubated with one or more peptidases to form a population of cleaved and non-cleaved peptides or proteins.

9. A method of building peptidase cleavable substrate library is provided, the method comprises the steps of:
(i) Providing an unbiased library of peptides or proteins, wherein the peptide or protein comprises a substrate sequence, and the design of the library is the result of mathematical modeling to assess all possible cooperative interactions between at least two, three or more amino acid residues within a given sequence space for a defined number of residues and length of substrate sequence;
(ii) Incubating the library in the presence of the peptidase, allowing the peptidase to cleave peptides or proteins within the library to form a population of cleaved and non-cleaved peptides or proteins;
(iii) Screening the population of cleaved and non-cleaved peptides or proteins for obtaining an amino acid motif(s) comprising a minimal set of two or more amino acids at varying positions within a peptidase enzyme substrate sequence that interact with the peptidase or peptidases (Gearr™ motifs);
(iv) Extracting the amino acid motifs using a computational algorithm; and
(v) Building a refined peptidase cleavable substrate sequence library from amino acid motifs, wherein the substrate sequences are capable of being cleaved by at least one peptidase.

10. A method of obtaining a peptidase cleavable substrate comprising the steps of:
(i) Building a peptidase cleavable substrate library according to the claim 9;
(ii) Selecting individual peptidase cleavable substrate comprising at least one amino acid motif, wherein the substrate is capable of being selectively cleaved by at least one, at least two, or at least three peptidases.

11. A method of obtaining disease-, tissue- and/or cell-selective peptidase cleavable substrate, comprising the steps of:
(i) Obtaining a peptidase cleavable substrate according to the claim 9;
(ii) Optionally, identifying bracketing residues, wherein bracketing residues are located at upstream and/or downstream positions outside of a candidate disease-, tissue- and/or cell-selective peptidase cleavable substrate;
(iii) Contacting the candidate peptidase cleavable substrate with at least one disease-, tissue- and/or cell-selective peptidase;
(iv) Evaluating and validating cleavage of the candidate substrate sequence, whereby obtaining the disease-, tissue- and/or cell-selective peptidase cleavable substrate.

12. The method of claim 9, wherein disease-, tissue- and/or cell-selective peptidase cleavable substrate is for use in therapeutic and/or diagnostic applications.

13. The method according claim 9, wherein the peptidases are selected from the group of endopeptidases, omega-peptidases, exopeptidases, aminopeptidases, carboxypeptidases, dipeptidyl-peptidases, tripeptidyl peptidases, peptidyl dipeptidases, dipeptidases, or combination thereof.

14. The method according to claim 13, wherein the peptidases are extracted from human subject-derived tissues and/or biofluids, cell cultures, transgenic cellular expression systems, or animal models for human disease and biological systems.

15. The method according to claim 1, wherein the screening of the population of cleaved and non-cleaved substrates comprises quantitively measuring the abundance of cleavage products at different time points by sequencing methods, wherein the sequencing methods are selected from mass spectrometry-based proteomic analysis, differential fluorescence, differential immunodetection, next generation sequencing techniques, or combination thereof.

16. The method according to the claim 15, further comprising analysis and identification of the scissile bond for each individual peptide by sequencing methods.

17. The method according to claim 1, wherein building a peptidase cleavable substrate library from the amino acid motifs comprises testing a candidate peptidase cleavable substrate within a sample, wherein the sample is selected based on a criteria matrix.

18. The method according to claim 17, wherein the criteria matrix guides sample selection.

19-21. (canceled)

22. A kit comprising one or more unbiased peptide libraries according to claim 1.

23. A peptide library comprising one or more unbiased peptides according to claim 1.
